(19) United States
(12) Patent Application Publication
(10) Pub. No.: US 2025/0099599 A1
(43) Pub. Date: Mar. 27, 2025

(54) FURIN-CLEAVABLE DELIVERY CONSTRUCTS

(71) Applicant: Thornhill Therapeutics, Inc., San Francisco, CA (US)

(72) Inventors: Thomas Carl Hunter, Mountain View, CA (US); Randall J. Mrsny, Los Altos Hills, CA (US); Geoffrey Masuyer, Järfälla (SE)

(21) Appl. No.: 18/777,927

(22) Filed: Jul. 19, 2024

Related U.S. Application Data
(63) Continuation of application No. PCT/US2023/060934, filed on Jan. 19, 2023.
(60) Provisional application No. 63/301,830, filed on Jan. 21, 2022.

(51) Int. Cl. A61K 47/64; C12N 9/10; C12N 9/64

(52) U.S. Cl. CPC A61K 47/64; C12N 9/1077; C12N 9/6408; C12Y 204/02031; C12Y 304/21075

(57) ABSTRACT

The present disclosure provides furin-cleavable delivery constructs that include a transcytosing element that is derived from a mono-ADP-ribosyl transferase and a heterologous cargo that is coupled to the carrier. The carrier is capable of facilitating transport of the heterologous cargo across an epithelial cell via transcytosis. The heterologous cargo may be released from a remaining portion of the delivery construct upon cleavage by a furin protease. The constructs may be used to facilitate delivery of a cargo to a basolateral side of an epithelial membrane.

### CROSS REFERENCE

This application is a continuation application of International Patent Application No. PCT/US2023/060934, filed Jan. 19, 2023, which claims the benefit of U.S. Provisional Application No. 63/301,830, filed Jan. 21, 2022, each of which is incorporated herein by reference in its entirety.

### SEQUENCE LISTING

The instant application contains a Sequence Listing which has been submitted electronically in XML format and is hereby incorporated by reference in its entirety. Said XML copy, created on Dec. 16, 2024, is named 67482-769.301_SL.xml and is 124,269 bytes in size.

### BACKGROUND OF THE INVENTION

Epithelial barriers have thwarted efforts for oral or respiratory administration of large therapeutic molecules such as proteins because proteins cannot diffuse across the intact epithelial barrier or cross the barrier through the tight junctions. Once taken up by an epithelial cell, a therapeutic protein typically either enters the destructive lysosomal trafficking pathway or, in the case of gut epithelial cells, is released back into the intestinal lumen. This inability to be readily transported across the epithelium continues to be a limiting factor in developing commercially viable oral and respiratory formulations, particularly for polypeptide-based therapeutics. A common solution is to use parenteral administration such as intravenous or subcutaneous administration, but these administration routes can often create considerable side effects, lower the therapeutic efficacy, and reduce patient convenience that can negatively affect compliance. There is a need for improved compositions and methods for transporting therapeutics into or across an epithelium, e.g., a gut epithelium.

### SUMMARY OF THE INVENTION

In some aspects, the present disclosure provides for a furin-cleavable delivery construct for delivery of a heterologous cargo across a polarized epithelial cell via transcytosis, the delivery construct comprising: a transcytosing element derived from a domain I of a mono-ADP-ribosyl transferase (mART); a heterologous cargo coupled to the transcytosing element; and a furin-cleavable cleavage site, wherein the furin-cleavable cleavage site has a scissile bond that is positioned between the transcytosing element and the heterologous cargo such that cleavage of the scissile bond by a furin protease releases the heterologous cargo from the transcytosing element. In some embodiments, the scissile bond of the furin-cleavable cleavage site is positioned such that, upon cleavage of the scissile bond by the furin protease, a released N-terminal fragment comprising the transcytosing element lacks a C-terminal portion of a domain I, lacks a domain II, and lacks a domain III of a mART. In some embodiments, the furin-cleavable cleavage site comprises a contiguous sequence of P4-P3-P2-P1-P1′-P2′-P3′-P4′, wherein P1 is Arg, wherein P2, P3, P4, P1′, P2′, P3′, and P4′ are each independently selected from any naturally occurring amino acid, and wherein the scissile bond of the cleavage site is positioned between P1-P1′. In some embodiments, P2 is Arg or Lys and P4 is Arg. In some embodiments, P3 is selected from the group consisting of histidine, glutamine, lysine, and glutamic acid. In some embodiments, P2 is Lys and P3 is His. In some embodiments, P3 is not Thr. In some embodiments, P1′ is not a tryptophan. In some embodiments, P1′ is Ser. In some embodiments, P1′ is Gly. In some embodiments, P1′ is His. In some embodiments, P2′ is selected from the group consisting of Val, Ala, Leu, Ile, Ser, Thr, and Lys. In some embodiments, P2′ is Gly. In some embodiments, P3′ is selected from the group consisting of Gly, Ala, Leu, Ser, Thr, Asp, Glu, and Arg. In some embodiments, P4′ is selected from the group consisting of Gly, Pro, Ala, Leu, Ser, Thr, Asp, Glu, Lys, and Arg. In some embodiments, P1′ is Ser, P2′ is Ala, P3′ is Ala, and P4′ is Gly. In some embodiments, P1′ is His, P2′ is Gly, P3′ is Glu, and P4′ is Gly. In some embodiments, the furin-cleavable cleavage site further comprises a contiguous sequence of P14-P13-P12-P11-P10-P9-P8-P7-P6-P5 positioned immediately N-terminal of P4, wherein P14 is Ser or Gly and wherein P9 is Gln or Pro. In some embodiments, the furin-cleavable cleavage site further comprises a contiguous sequence of P14-P13-P12-P11-P10-P9-P8-P7-P6-P5 positioned immediately N-terminal of P4, wherein P14 is Ser and wherein P9 is Gln. In some embodiments, the contiguous sequence of P14-P13-P12-P11-P10-P9-P8-P7-P6-P5 comprises SYKAAQKEGS. In some embodiments, the contiguous sequence of P14-P13-P12-P11-P10-P9-P8-P7-P6-P5 comprises SVVMAQAQPR. In some embodiments, at least 50% the delivery construct is cleaved by furin after 18 h at room temperature under the following conditions: 2.5 ug of delivery construct, 2 μg of furin, 20 mM HEPES at pH 5, 0.2 mM CaCl₂, 0.2 mM TCEP in a total of between 25 and 30 microliters. In some embodiments, the scissile bond is positioned between amino acid residues 186 and 260 of SEQ ID NO: 43 when the transcytosing element and the furin-cleavable cleavage site are optimally aligned with SEQ ID NO: 43. In some embodiments, the scissile bond is positioned between amino acid residues 186 and 248 of SEQ ID NO: 44 when the transcytosing element and the furin-cleavable cleavage site are optimally aligned with SEQ ID NO: 44. In some embodiments, the delivery construct colocalizes with furin in an endosome during transcytosis, but is not cleaved by furin within the endosome at a stage that prevents basolateral release of the heterologous cargo. In some embodiments, the delivery construct is cleaved by furin upon exit from the polarized epithelial cell. In some embodiments, the heterologous cargo comprises a protein therapeutic or peptide therapeutic. In some embodiments, the construct further comprises a linker positioned between the furin cleavable cleavage site and the heterologous cargo. In some embodiments, the linker comprises or consists of glycine and serine residues. In some embodiments, the linker comprises the amino acid sequence set forth in any one of SEQ ID NOS: 26-32. In some embodiments, the linker is coupled to and positioned N-terminal of the cargo upon cleavage of the scissile bond by the furin protease. In some embodiments, the transcytosing element has at least 80%, at least 85%, at least 90%, at least 95%, at least 98%, at least 99%, or 100% identity to a transcytosing element from a domain I of a Vibrio cholerae, Pseudomonas aeruginosa, Aeromonas, Chromobacterium, Collimonas, Shewanella, Janthinobacterium, Serratia, or Acinetobacter mART. In some embodiments, the domain I of Vibrio cholerae is SEQ ID NO: 43, the domain I of Pseudomonas aeruginosa is SEQ ID NO: 44, the domain I of Aeromonas is SEQ ID NO: 45, the domain I of Chromobacterium is SEQ ID NO: 46, the domain I of Collimonas is SEQ ID NO: 47, the domain I of Shewanella is SEQ ID NO: 48, the domain I of Janthinobacter is SEQ ID NO: 49, the domain I of Serratia is SEQ ID NO: 50, and the domain I of Acinetobacter is SEQ ID NO: 51. In some embodiments of any of the aforementioned aspects or embodiments, the delivery construct lacks mono-ADP-ribosyl transferase activity. In some embodiments of any of the aforementioned aspects or embodiments, the delivery construct lacks a domain III of a mART. In some embodiments of any of the aforementioned aspects or embodiments, the delivery construct lacks a domain II of a mART. In some embodiments of any of the aforementioned aspects or embodiments, when the transcytosing element is optimally aligned with the domain I of the mART from which it is derived, P1, P2, P3, and P4 are each identical to the amino acid to which they are aligned. In some embodiments of any of the aforementioned aspects or embodiments, the scissile bond is positioned C-terminal of a core 12-14-stranded β-jellyroll fold of the transcytosing element. In some embodiments of any of the aforementioned aspects or embodiments, when the delivery construct is optimally aligned with SEQ ID NO: 43, the scissile bond of the delivery construct is C-terminal of an amino acid that is aligned with the Lys at position 186 of SEQ ID NO: 43. In some embodiments of any of the aforementioned aspects or embodiments, when the delivery construct is optimally aligned with the domain I of the mART, the scissile bond of the delivery construct is N-terminal of an amino acid that is aligned with the Arg at position 260 of SEQ ID NO: 43. In some embodiments of any of the aforementioned aspects or embodiments, when the delivery construct is optimally aligned with the domain I of the mART, the scissile bond of the delivery construct is N-terminal of an amino acid that is aligned with the Ile at position 261 of SEQ ID NO: 43. In some embodiments of any of the aforementioned aspects or embodiments, the transcytosing element comprises or consists of SEQ ID NO: 20, and the scissile bond is immediately C-terminal of position 197. In some embodiments, the transcytosing element comprises or consists of SEQ ID NO: 52, and the scissile bond is immediately C-terminal of position 186. In some embodiments of any of the aforementioned aspects or embodiments, the transcytosing element comprises or consists of any one of SEQ ID NO: 20 and SEQ ID NOs: 52-61 or a sequence having at least 80%, 85%, 90%, 95%, 97%, 98%, or 99% sequence identity thereto, and the scissile bond is immediately C-terminal of the transcytosing element. In some embodiments of any of the aforementioned aspects or embodiments, the transcytosing element comprises or consists of any one of SEQ ID NO: 20 and SEQ ID NOs: 52-61, and the scissile bond is immediately C-terminal of the transcytosing element. In some embodiments of any of the aforementioned aspects or embodiments, the released N-terminal fragment comprises or consists of any one SEQ ID NO: 20, SEQ ID NO: 60, or SEQ ID NO: 61. In some embodiments, the released N-terminal fragment comprises or consists of SEQ ID NO: 52. In some embodiments of any of the aforementioned aspects or embodiments, the heterologous cargo comprises any one of SEQ ID NOS: 35-38. In some embodiments, the heterologous cargo comprises SEQ ID NO: 62. In some embodiments of any of the aforementioned aspects or embodiments, the delivery construct comprises: a carrier consisting of SEQ ID NO: 20, SEQ ID NO: 60, SEQ ID NO: 61, or SEQ ID NO: 52, and optionally an N-terminal methionine; and a linker between the transcytosing element and the heterologous cargo, wherein the linker comprises the amino acid sequence set forth in any one of SEQ ID NOS: 26-32. In some embodiments, the carrier consists of SEQ ID NO: 20, SEQ ID NO: 60, or SEQ ID NO: 61, and optionally an N-terminal methionine. In some embodiments, the linker comprises SEQ ID NO: 30. In some aspects, the present disclosure provides for a method of delivering a cargo across an epithelial membrane, the method comprising: delivering the furin-cleavable delivery construct of any preceding claim to an apical side of an epithelial membrane such that the cargo (1) transcytoses across an epithelial cell of the epithelial membrane and (2) is cleaved from the transcytosing element by a furin protease to deliver the cargo to a basolateral side of the epithelial membrane. In some embodiments, the epithelial cell is a gut epithelial cell. In some embodiments, the epithelial cell is a polarized epithelial cell.

In some aspects, the present disclosure provides for a nucleic acid comprising (a) a polynucleotide sequence encoding any of the furin-cleavable delivery described herein; or (b) a reverse complement of said polynucleotide sequence of (a).

In some aspects, the present disclosure provides for an expression vector comprising any of the polynucleotide sequences described herein and a recombinant regulatory sequence operably linked to said polynucleotide sequence.

In some aspects, the present disclosure provides for a host cell, comprising any of the expression vectors described herein. In some embodiments, the host cell is a prokaryote.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides an SDS-reducing gel showing separation of various components of delivery constructs upon treatment with furin.

FIG. 2A is a total ion chromatogram (TIC) of the delivery construct Chx¹⁹⁷-SAAG-N3-hGH.

FIGS. 2B and 2C provide a mass spectrum of the delivery construct Chx¹⁹⁷-SAAG-N3-hGH remains intact.

FIG. 3A is a TIC showing the fragments of delivery construct Chx¹⁹⁷-SAAG-N3-hGH after furin digestion for 5 hours.

FIGS. 3B and 3C provide a mass spectrum showing the fragments of delivery construct Chx¹⁹⁷-SAAG-N3-hGH after furin digestion for 5 hours.

FIG. 4A is a TIC showing the fragments of delivery construct Chx¹⁹⁷-SAAG-N3-hGH after furin digestion for 24 hours.

FIGS. 4B and 4C provide a mass spectrum showing the fragments of delivery construct Chx¹⁹⁷-SAAG-N3-hGH after furin digestion for 24 hours.

FIG. 5 is a Western blot showing the extent of furin-mediated cleavage of PE188-exenatide and Cholix¹⁹⁷-exenatide.

FIG. 6A is a TIC of exenatide.

FIGS. 6B and 6C provide a mass spectrum of exenatide.

FIG. 7A is a TIC of Chx¹⁹⁷-exenatide.

FIGS. 7B and 7C provide a mass spectrum of Chx¹⁹⁷-exenatide.

FIG. 8A is a TIC showing the fragments of delivery construct Chx¹⁹⁷-exenatide after furin digestion.

FIGS. 8B and 8C provide a mass spectrum showing the fragments of delivery construct Chx¹⁹⁷-exenatide after furin digestion.

FIGS. 9A, 9B, and 9C are the TIC and the extracted ion chromatograms (XICs) of FIG. 8A between 0 and 6 minutes.

FIG. 9D provide a mass spectrum of FIGS. 8B and 8C between 0 and 27000 Da.

FIG. 10A is a TIC of Chx¹⁹⁷-exenatide.

FIG. 10B provides a mass spectrum of Chx¹⁹⁷-exenatide.

FIG. 11A is a TIC showing the fragments of delivery construct PE188-exenatide after furin digestion.

FIGS. 11B and 11C provides a mass spectrum showing the fragments of delivery construct PE188-exenatide after furin digestion.

FIGS. 12A, 12B, and 12C are the TIC and the extracted ion chromatograms (XICs) of FIG. 11A between 0 and 6 minutes.

FIG. 12D provides the mass spectrum of FIGS. 11B and 11C between 0 and 27000 Da.

FIG. 13A is a Western blot showing the extent of furin-mediated cleavage of various delivery constructs.

FIGS. 13B and 13C show ELISA-based graphs of extent of transport of three cholix-hGH proteins across AIR-100 cells.

FIGS. 14A, 14B, 15A, 15B, 16A, and 16B are bar graphs quantifying aspects of the image of FIG. 13A.

FIG. 17 provides Western blots showing cleavage of various delivery constructs upon transport to the basolateral solution in in vitro experiments.

FIG. 18 is a bar graph quantifying the relative extent of transport of various delivery constructs to the basolateral solution in in vitro experiments.

FIG. 19 is a Western blot showing the extent of furin-mediated cleavage of Chx¹⁹⁷-SAAG-N3-hGH.

FIG. 20 provides Western blots comparing the extent of cleavage of Chx¹⁹⁷-SAAG-N3-hGH upon transport to the basolateral solution with the furin-mediated cleavage of Chx¹⁹⁷-SAAG-N3-hGH.

FIGS. 21A, 21B, 21C, and 21D provide immunofluorescence images of various delivery constructs.

FIGS. 22A, 22B, 22C, 22D, 22E, 22F, 22G, 22H, 22I, 22J, and 22K provide immunofluorescence images of Chx¹⁹⁷-GGG-hGH at different time points.

FIG. 23 is a graph showing serum concentrations of human growth hormone (hGH) over time upon in vivo administration of various delivery constructs to rats.

FIG. 24 is a bar graph quantifying the AUC of FIG. 23.

FIG. 25 is a graph showing serum concentrations of human growth hormone (hGH) over time upon in vivo administration of various delivery constructs to rats.

FIG. 26 is a bar graph quantifying the AUC of FIG. 25.

FIG. 27 is a Western blot assessing hGH levels in the serum of rats that have been administered various delivery constructs.

FIG. 28 is a Western blot assessing cholix carrier levels in the serum of rats that have been administered various delivery constructs.

FIGS. 29A, 29B, 29C, and 29D are charts showing serum levels of human growth hormone over time in individual rats that have been administered various delivery constructs.

FIG. 30 is a Western blot showing limited release of human growth hormone cargo from a delivery construct.

FIG. 31 provide a Western blot showing the extent of furin mediated cleavage of various delivery constructs.

FIG. 32 is a chart showing the prevalence of various amino acids at positions flanking the scissile bond of furin cleavage sites as assessed using the MEROPS database (www.ebi.ac.uk/merops/).

FIGS. 33A and 33B provide a sequence alignment of a portion of various mono-ADP-ribosyltransferases (mARTs).

### DETAILED DESCRIPTION OF THE INVENTION

In the following description, certain specific details are set forth in order to provide a thorough understanding of various embodiments. However, one skilled in the art will understand that the embodiments provided may be practiced without these details.

### I. Introduction

Provided herein, in certain embodiments, are transcytosing elements and delivery constructs (e.g., transcytosing element-payload conjugates) capable of transporting one or more heterologous payload molecules (e.g., one or more therapeutic payloads) across epithelial cells (e.g., polarized gut or respiratory epithelial cells) by transcytosis. The delivery constructs can comprise a transcytosing element that is coupled to the heterologous payload. The transcytosing element may be genetically coupled to the heterologous payload, or may be chemically coupled to the heterologous payload. The transcytosing element can be capable of transporting the heterologous payload into or across epithelial cells using endogenous trafficking pathways. Utilization of endogenous trafficking pathways, as opposed to use of passive diffusion, can allow the transcytosing element to shuttle the heterologous payload rapidly (e.g., at least 10⁻⁶ cm/sec, 10⁻⁵ cm/sec) and efficiently (e.g., at least 5%, 10%, 20%, 25%, or 50% of material applied to the apical surface) into or across epithelial cells without impairing the barrier function of these cells or the biological activity of the heterologous payload.

The delivery constructs described herein can include a furin-cleavable cleavage site having a scissile bond that is positioned between the transcytosing element and the heterologous cargo such that cleavage of the scissile bond by a furin protease releases the heterologous cargo from the transcytosing element. The scissile bond of the furin-cleavable cleavage site may be positioned such that, upon cleavage of the scissile bond by the furin protease, a released N-terminal fragment comprising the transcytosing element lacks (1) a C-terminal portion of domain I, (2) domain II, and (3) domain III. In other embodiments, the carrier is positioned C-terminal of the scissile bond of the furin-cleavable cleavage site while a released C-terminal fragment comprises the transcytosing element.

### II. Certain Definitions

Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

A “furin-cleavable cleavage site” is a site that is cleavable by a furin protease. In some embodiments, the furin-cleavable cleavage site of the delivery construct is a site containing a scissile bond, where at least 5%, 10%, 20%, 30%, 40%, 50%, 60%, 70%, 80%, 90% or 95% of the delivery construct is cleaved at a scissile bond under certain specified conditions. In some embodiments, such specified conditions may the conditions set forth in Example 2 for a specified period of time, such as 18 hours. In some embodiments, a cleavage site within the delivery construct may be cleavable by a proprotein convertase, such as furin. In some embodiments, the cleavage site that is cleavable by the proprotein convertase is cleavable by a furin protease, while in other embodiments the cleavage site that is cleavable by a proprotein convertase is not cleavable by a furin protease but is cleavable by some other proprotein convertase.

### III. Transcytosing Elements

The transcytosing element (also referred to as a carrier) of a delivery construct provided herein can be any molecule or protein sequence that is capable of increasing the rate and/or amount of a heterologous payload (e.g., a therapeutic payload) delivered into and/or across an epithelium.

A transcytosing element herein can have any of various attributes. In some embodiments, a transcytosing element herein can be derived from a mono-ADP-ribosyltransferase (mART). In some embodiments, a transcytosing element can be a portion of a mART with a reduced (e.g., at least 50% reduced) or ablated ADP ribosylation activity (e.g., ribosylation of elongation factor 2) relative to a naturally occurring exotoxin. In some embodiments, the transcytosing element is a mART that has been truncated and/or mutated relative to a naturally occurring mART in a manner that results in reduced or ablated ADP ribosylation activity.

### (a) Transcytosing Elements May Utilize Endogenous Trafficking Pathways

In some embodiments, a transcytosing element herein utilizes an endogenous trafficking pathway to transport a heterologous payload coupled thereto across a polarized epithelial cell. In some instances, a transcytosing element herein can utilize an endogenous trafficking pathway to transport a heterologous payload coupled thereto across a polarized epithelial cell via transcytosis.

Any of the transcytosing elements herein can transport molecules coupled thereto by interacting and/or co-localizing with one or more endogenous proteins of such epithelium. The one or more endogenous proteins can be receptors or enzymes capable of moving a transcytosing element into or across the epithelial cell. Interacting and/or co-localizing with the one or more endogenous proteins of the epithelial cell can provide a transcytosing element with one or more functions, including endocytosis into the epithelial cell, avoidance of a lysosomal destruction pathway, trafficking from an apical compartment to a basal compartment, and/or exocytosis from the basal membrane of the epithelial cell into a submucosal compartment such as the lamina propria.

An interaction of such transcytosing element with an endogenous protein can be a selective interaction. Such selective interaction can be a pH-dependent interaction. In instances where a transcytosing element interacts with two or more endogenous proteins, such interactions can be sequential interactions where a first interacting protein hands the transcytosing element off to a second interacting protein. Such sequential interactions can occur at a different pH (e.g., pH 5.5, 7.0, 7.5, etc.). An interaction between a transcytosing element and an endogenous protein can be a covalent or non-covalent interaction. Non-covalent interactions include hydrogen bonding, van der Waals interactions, ionic bonds, π-π-interactions, etc.

In some instances, one of the endogenous proteins that a transcytosing element can interact with can be an apical entry receptor. Interaction of a transcytosing element with such apical entry receptor can enable the transcytosing element to initially enter the epithelial cell through receptor-mediated endocytosis.

A transcytosing element can also interact with a lysosome avoidance receptor. Such interaction with a lysosome avoidance receptor can occur inside the epithelial cell and subsequent to endocytosis. Interaction of a transcytosing element with a lysosome avoidance receptor can enable the transcytosing element to avoid or circumvent lysosomal degradation. Such ability can allow a transcytosing element to significantly reduce the amount of payload coupled to the transcytosing element reaching a lysosome of a cell, a fate that therapeutic proteins can face once taken up by the gut epithelium.

Furthermore, a transcytosing element can interact with an apical to basal trafficking protein. Such interaction can occur inside the epithelial cell and subsequent to endocytosis. Interaction of a transcytosing element with a basal trafficking protein can enable the transcytosing element to move from an apical compartment to a supranuclear compartment or a basal compartment.

A transcytosing element can also interact with a basal release protein capable of promoting exocytosis of a transcytosing element from a basal site of an epithelial cell. Such interaction can occur at the basal site of an epithelial cell and subsequent to moving from an apical compartment to a basal compartment. Interaction of a transcytosing element with a basal release protein can enable the transcytosing element to access a basal recycling system that allows release of the transcytosing element from the basal compartment into a submucosal compartment such as the lamina propria.

Thus, a transcytosing element herein can be a molecule that is capable of interacting with endogenous proteins, enabling such transcytosing element to transport a payload molecule coupled thereto across a polarized epithelium, e.g., a polarized gut or respiratory epithelium.

A transcytosing element can be a polypeptide. Such transcytosing element can be derived from a polypeptide secreted by a bacterium, such as the mART of Vibrio cholerae, Pseudomonas aeruginosa, Aeromonas, Chromobacterium, Collimonas, Shewanella, Janthinobacterium, Serratia, or Acinetobacter.

A transcytosing element can be a naturally or non-naturally occurring polypeptide of a polypeptide secreted by such bacterium.

Non-naturally occurring polypeptides can include those having a C- and/or an N-terminal modification.

In one example, a polypeptide comprises one or more amino acid substitutions, and/or one or more amino acid deletions, and/or one or more amino acid additions relative to a sequence alignment with a naturally occurring polypeptide or relative to a sequence alignment with a consensus sequence.

Examples of substitutions contemplated herein include conservative substitutions of one or more amino acids. The following six groups each contain amino acids that are conservative substitutions for one another: (1) Alanine (A), Serine(S), and Threonine (T); (2) Aspartic acid (D) and Glutamic acid (E); (3) Asparagine (N) and Glutamine (Q); (4) Arginine (R) and Lysine (K); (5) Isoleucine (I), Leucine (L), Methionine (M), and Valine (V); and (6) Phenylalanine (F), Tyrosine (Y), and Tryptophan (W).

Conservative substitutions further include substitutions within the following groups: aliphatic residues (I, V, and L); aromatic residues (Y, H, W, and F); hydrophobic residues (W, F, Y, M, L, I, V, A, C, T, and H); alcohol residues (S and T); polar residues (D, E, H, K, N, Q, R, S, and T), tiny residues (A, G, C, and S); small residues (A, G, C, S, V, N, D, T, and P); bulky residues (E, F, I, K, L, M, Q, R, W, and Y); positively charged residues (K, R, and H); negatively charged residues (D and E); and charged residues (D, E, K, R, and H).

Examples of deletions include N-terminal truncations, C-terminal truncations, and internal deletions.

Examples of additions include: a signal peptide sequence, a purification peptide sequence, or other N-terminal modifications. A signal peptide sequence can comprise 1 to about 40 amino acids. In some cases, a transcytosing element comprises an N-terminal methionine (e.g., to facilitate expression in a bacterial host). The term “about,” as used herein in the context of a numerical value or range, generally refers to +10% of the numerical value or range recited or claimed, unless otherwise specified.

A transcytosing element can have a substantial sequence identity (e.g., about or greater than 50%, 60%, 70%, 80%, 85%, 90%, 95%, 98% or 99% sequence identity, or 100% sequence identity) to a naturally occurring sequence of polypeptide, such as a domain I (or portion thereof) of a Vibrio cholerae, Pseudomonas aeruginosa, Aeromonas, Chromobacterium, Collimonas, Shewanella, Janthinobacterium, Serratia, or Acinetobacter mART. For instance, the transcytosing element can have at least 80%, at least 85%, at least 90%, at least 95%, at least 98%, at least 99%, or 100% identity to a transcytosing element to a domain I of Pseudomonas aeruginosa (SEQ ID NO: 44), a domain I of Aeromonas (SEQ ID NO: 45), a domain I of Chromobacterium (SEQ ID NO: 46), a domain I of Collimonas (SEQ ID NO: 47), a domain I of Shewanella (SEQ ID NO: 48), a domain I of Janthinobacter (SEQ ID NO: 49), a domain I of Serratia (SEQ ID NO: 50), or a domain I of Acinetobacter (SEQ ID NO: 51).

The term “sequence identity” or a percent (%) of sequence identity, as used herein can be the percentage of residues in a candidate sequence that are identical with the residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

N-terminal truncations include those that remove up to 10, 20, 30, 39, or 40 amino acids at the N-terminus of a mART sequence herein or the N-terminus of Domain I of a mART sequence herein. C-terminal truncations can be those described herein. Such N- and/or C-terminal truncations can result in different functions. Truncations can be described as relative to a naturally occurring sequence or portion thereof (e.g., the sequence of SEQ ID NO: 3), or relative to a consensus sequence (e.g., the sequence of SEQ ID NO: 42). As used herein, residues are numbered from the N-terminus to the C-terminus, starting with position 1 at the N-terminus unless the sequence begins with an N-terminal methionine, in which case the sequence begins with amino acid positioned C-terminal of the N-terminal methionine.

A transcytosing element herein can be derived from a domain I polypeptide from (e.g., secreted from) Vibrio cholerae, Pseudomonas aeruginosa, Aeromonas hydrophila, Chromobacterium haemolyticum, Collimonas fungivorans, Shewanella putrefacians, Janthinobacterium lividum, Serratia fonticola, or Acinetobacter baumannii bacterium (e.g., any one of SEQ ID NOS: 43-51. A transcytosing element derived from a mART polypeptide can include naturally and non-naturally occurring mART polypeptide sequences, as well as those sequences that have at least about 75%, 80%, 85%, 90%, 95%, 98%, 99%, or 100% sequence identity to a naturally occurring mART polypeptide (e.g., the sequence set forth in any one of SEQ ID NOs: 3 and 20) described herein.

(b) Mono-ADP-Ribosyl Transferases (mARTs)

A mono-ADP-ribosyl transferase (mART) is a polypeptide that typically contains at least three domains: DI, DII, and DIII. Domain I is typically located at the N-terminus of the mature protein and Domain III is typically located at the C-terminus. A mART can have a leader (or signal) peptide at its N-terminus. In its native setting, the leader peptide may target the mART for secretion. A leader peptide is removed from the mature protein by an endopeptidase. A mART may further comprise a Domain Ib domain that associates with Domain I in a folded, three-dimensional structure, although it is not continuous with the DI domain in the amino acid sequence of the mART. The total size of a mART can be 600-650 amino acids.

Domain I (DI) of a mART can function as a carrier for endocytosis and/or transcytosis. It can comprise a core 12-14 (e.g., 13)-stranded beta-jellyroll fold structure. Each beta-strand of DI is linked to the next by a loop region. Domain I can be about 250-270 amino acids in length. A functional Domain I can comprise at least 150 amino acids. Domain I may start after a leader peptide cleavage site (or at an N-terminus in the case where there is no leader peptide cleavage site) and may end at a HFxxx sequence or, preferably, an HFxxG or HFxxE sequence. Domain I may end at the end of a HFxxx, HFxxG or HFxxE sequence or 1, 2, or 3 amino acids from the end of a HFxxx, HFxxG or HFxxE sequence.

Domain I may comprise amino acids that bind to a protein, glycoprotein, or glycolipid displayed on the surface of an epithelial cell and/or a cell sensitive to intoxication by the mART. Binding of a Domain I to a cell surface protein, glycoprotein, or glycolipid can mediate entry of the mART into the cell. Domain I of a mART may enter a cell by endocytosis, micropinocytosis, or macropinocytosis and it may enter via calveolae or clathrin-coated pits. Domain I can further have transcytosis carrier activity. Domain I may comprise amino acids that interact with intracellular proteins in endosomal compartments or membranes to mediate transport within apical endosomes, to avoid trafficking to lysosomes, to direct transport from an apical endosome or a perinuclear vesicle to a basal or basolateral endosome, and to be released from an epithelial cell by exocytosis of a basolateral vesicle.

Domain II (DII) of a mART can form a six alpha helix bundle. Domain II can mediate translocation of DIII of a mART across an intracellular membrane. The intracellular membrane can be an endoplasmic reticulum membrane.

Domain III (DIII) of a mART can have transferase activity. In some embodiments, transferase activity is mediated by a catalytic glutamic acid residue. The substrate for the transferase may be elongation factor Tu (EF-Tu). In some embodiments, Domain III of a mART does not comprise a catalytic glutamic acid residue, either naturally, or by design to reduce toxicity. In some embodiments, Domain III comprises a retrograde transport signal that mediates intracellular transport of the mART to the endoplasmic reticulum.

A mART may comprise a transcytosing element. A transcytosing element is any portion of a mART that mediates transcytosis of the mART across a polarized epithelial cell. A transcytosing element may comprise all or part of Domain I of the mART. The transcytosing element may be a Domain I of a mART with an N-terminal, C-terminal, or internal deletion. The transcytosing element can mediate transcytosis across a gut epithelial cell or a respiratory epithelial cell. The transcytosing element can mediate transcytosis across a polarized SMI-100 cell, Caco-2 cell, AIR-100 cell, or MDCK cell.

In some embodiments, a transcytosing element of a mART can comprise or consist of (1) a sequence set forth in any of SEQ ID NOs: 1-6, 20, or 42-51 (see Table 1), (2) portion of a sequence set forth in any of SEQ ID NOs: 1-6, 20, or 42-51 (e.g., SEQ ID NOs: 52-61), or a (3) a sequence having at least 80%, 85%, 90%, 95%, 97%, 98%, 99%, or 100% sequence identity to (1) or (2). In some embodiments, the transcytosing element comprises or consists of SEQ ID NO: 20. In some embodiments, the transcytosing element comprises or consists of SEQ ID NO: 20 or a modified sequence that replaces the VEDE sequence therein with VEEA (SEQ ID NO: 60) or LEEA (SEQ ID NO: 61). In some embodiments, the transcytosing element comprises or consists of any one of SEQ ID NOs: 52-61, or a modified sequence that replaces the first four amino acids with VEDE, VEEA, or LEEA. In some embodiments, the transcytosing element has at least 80%, 85%, 90%, 95%, 97%, 98%, 99%, or 100% sequence identity to any one of SEQ ID NOS: 20 or 52-61. In some embodiments, the transcytosing element comprises or consists of the first 197 amino acids of SEQ ID NO: 2 (SEQ ID NO: 61), optionally further including an N-terminal methionine.

In some embodiments, the transcytosing element comprises or consists of a portion of sequence of any of SEQ ID NOs: 1-6, 20, or 42-51, wherein the portion of the sequence is at least 180, 185, 190, 195, or 197 amino acids in length. In some embodiments, the transcytosing element comprises or consists of a portion of the sequence of any of SEQ ID NOs: 1-6, 20, or 42-51, wherein the portion of the sequence is no more than 265, 250, 240, 220, 210, 200, or 197 amino acids in length. In some embodiments, the transcytosing element comprises or consists of a sequence having at least 80%, 85%, 90%, 95%, 97%, 98%, 99%, or 100% sequence identity to a portion of a sequence of any of SEQ ID NOs: 1-6, 20, or 42-51, wherein the portion of the sequence is at least 180, 185, 190, 195, or 197 amino acids in length. In some embodiments, the transcytosing element comprises or consists of a sequence having at least 80%, 85%, 90%, 95%, 97%, 98%, 99%, or 100% sequence identity to a portion of a sequence of any of SEQ ID NOs: 1-6, 20, or 42-51, wherein the portion of the sequence is no more than 265, 250, 240, 220, 210, 200, or 197 amino acids in length. The transcytosing element may have a C-terminus that is positioned immediately adjacent to the scissile bond.

In some embodiments, the transcytosing element is the only portion of the furin-cleavable delivery construct that is derived from a mART. In some embodiments, all mART-derived portions of the delivery construct are considered to be the transcytosing element. The transcytosing element may include one or more mutations, insertions, or deletions relative to the mART sequence from which it was derived.

In some embodiments, the furin-cleavable delivery construct comprises an N-terminal methionine (e.g., to facilitate expression in bacteria). In some embodiments, the N-terminal methionine is positioned immediately N-terminal of the transcytosing element. In some embodiments, a signal peptide, a purification tag (e.g., a His6 Tag) or some other peptide sequence may be positioned N-terminal of (e.g., immediately N-terminal of) the transcytosing element. In some embodiments, an N-terminal methionine is positioned N-terminal of (e.g., immediately N-terminal of) a signal peptide or a purification tag.

In some embodiments, the transcytosing element has a leucine residue at position 1 of the transcytosing element, which may reduce or eliminate cleavage of an amino acid (e.g., methionine) positioned N-terminal of the transcytosing element.

### IV. Payloads

In addition to the transcytosing element, the compositions provided herein can comprise one or more payloads, e.g., one or more heterologous payloads, or one or more biologically-active payloads, for delivery to a subject. The one or more heterologous payloads can be one or more payloads that do not have a transcytosing element, such as payloads that lack a transcytosing portion of a mART sequence. The one or more payloads, e.g., one or more heterologous payloads, can be a macromolecule, small molecule, small organic molecule, peptide, polypeptide, nucleic acid, mRNA, miRNA, shRNA, siRNA, PNA, antisense molecule, antibody (e.g., antibody fragment, scFv, single domain antibody, diabody, camelids, nanobody), DNA, plasmid, polysaccharide, lipid, antigen, vaccine, polymer nanoparticle, or catalytically-active material.

The one or more payloads, e.g., one or more heterologous payloads, e.g., one or more biologically active payloads, can be a macromolecule that can perform a desirable biological activity after transport across an epithelial cell and/or when introduced to the bloodstream of the subject. For example, the one or more payloads can have receptor binding activity, enzymatic activity, messenger activity (i.e., act as a hormone, cytokine, neurotransmitter, clotting factor, growth factor, or other signaling molecule), luminescent or other detectable activity, or regulatory activity, or any combination thereof. In various diagnostic embodiments, the one or more payloads can be conjugated to or can itself be a pharmaceutically acceptable gamma-emitting moiety, including but not limited to, indium and technetium, magnetic particles, radiopaque materials such as air or barium and fluorescent compounds (e.g., Alexa-488 or a red fluorescent protein). In some cases, the one or more payloads, e.g., one or more biologically active payloads, do not enter the bloodstream of the subject. In some cases, the one or more payloads, e.g., one or more biologically active payloads, enter the bloodstream of the subject. In some cases, the one or more payloads act at the lamina propria.

In various embodiments, the one or more payloads is a protein that comprises more than one polypeptide subunit. For example, the protein can be a dimer, trimer, or higher order multimer. In various embodiments, two or more subunits of the protein can be connected with a covalent bond, such as, for example, a disulfide bond. In other embodiments, the subunits of the protein can be held together with non-covalent interactions. One of skill in the art can identify such proteins and determine whether the subunits are properly associated using, for example, an immunoassay.

In some cases, one or more payloads, e.g., one or more heterologous payloads, used with the methods and compositions disclosed herein can be a hormone. Examples of hormones include, but are not limited to, human growth hormone, synthetic human growth hormone, human growth hormone 2. The one or more payloads, e.g., one or more heterologous payloads, can be a polypeptide comprising, consisting of, or consisting essentially of the sequence set forth in SEQ ID NO: 35 or a sequence with 85%, 90%, 95% or 98% identity thereto.

In various embodiments, the one or more payloads, e.g., one or more therapeutic payloads, are for example a dye, a radiopharmaceutical, a hormone, a cytokine, an anti-TNF agent, an antineoplastic compound, an agent for the treatment of hemophilia, an enzyme, a glucose lowering agent, insulin, or an insulin analog, or a derivative of insulin, or a tumor associated antigen. In some embodiments, the one or more payloads, e.g., one or more heterologous payloads, can be a polypeptide comprising, consisting of, or consisting essentially of the sequence set forth in SEQ ID NO: 62 or a sequence with 85%, 90%, 95% or 98% identity thereto.

In some cases, the one or more therapeutic payloads is a polypeptide that is a modulator of inflammation in the GI tract. In various embodiments, the one or more payloads is a glucose-lowering agent for delivery to a subject. In various embodiments, the one or more payloads is a one or more incretins or an incretin mimetic. An incretin can be glucagon-like peptide-1 (GLP-1), Gastric inhibitory peptide (GIP), or a GIP/GLP-2 hybrid.

In some cases, heterologous cargo comprises or consists of the sequence of any one of SEQ ID NOS: 35-38, or a sequence with 85%, 90%, 95%, or 98% identity thereto. In some cases, heterologous cargo comprises or consists of the sequence of SEQ ID NO: 62, or a sequence with 85%, 90%, 95%, or 98% identity thereto.

### V. Delivery Constructs

Provided herein are delivery constructs (e.g., transcytosing element-payload conjugates) that can comprise a transcytosing element coupled to one or more heterologous payloads. A transcytosing element can be coupled to the one or more heterologous payloads in a covalent fashion. A transcytosing element can be coupled directly or indirectly (e.g., through one or more linker sequences) to the one or more heterologous payloads.

The one or more heterologous payloads can be coupled to an N- and/or C-terminus of a transcytosing element. In some instances, the one or more heterologous payloads is directly and covalently coupled to a C-terminus of a carrier by forming a covalent amide bond between the C-terminal carboxyl group of the carrier and the N-terminal amine of a heterologous payload. In some instances, the one or more heterologous payloads is indirectly and covalently coupled to the carrier via a spacer or linker sequence.

Thus, in some instances, when a transcytosing element is covalently coupled to a payload, the delivery construct can be represented according to Formula II: T-L-P or Formula III: P-L-T, wherein T is a transcytosing element, L is a linker (spacer), or optionally a bond, and P is a heterologous payload. A delivery construct can further comprise one or more modifications on its N-terminus and/or C-terminus. Such a modification(s) can include an N-terminal methionine residue. Thus, Formula II and Formula III can also include an N-terminal methionine (e.g., M+T-L-P) or (e.g., M+P-L-T).

In some cases, the linker sequence is positioned between a furin-cleavable cleavage site and the heterologous cargo. In some embodiments, the linker may comprise or consist of (1) glycine residue(s) or (2) glycine and serine residues. In some embodiments, the linker comprises the amino acid sequence set forth in any one of SEQ ID NOS: 26-32. In some instances, the linker may be coupled to and positioned N-terminal of the heterologous cargo upon cleavage of the scissile bond by a furin protease.

In some embodiments, the delivery construct comprises (1) a carrier consisting of SEQ ID NO: 20 (optionally further including an N-terminal methionine), (2) a linker between the transcytosing element and (3) the heterologous cargo, wherein the linker comprises an amino acid sequence set forth in any one of SEQ ID NOS: 26-32, such as SEQ ID NO: 30.

A carrier can be coupled to one or more heterologous payloads via chemical/synthetic conjugation (e.g., using amide coupling reactions) or by recombinant expression, e.g., in a bacterial (e.g., in E. coli) or mammalian (e.g., Chinese Hamster Ovary (CHO)) cell as a fusion protein.

A delivery construct, or part thereof (e.g., the carrier and/or spacer), can be a polypeptide. The term “polypeptide,” as used herein, can include both natural and unnatural amino acids.

Furthermore, a transcytosing element can transport a payload across an intact epithelium (e.g., a polarized gut epithelium) with transport rates of at least about 10⁻⁶ cm/sec, 10⁻⁵ cm/sec, or 10⁻⁴ cm/sec.

### VI. Furin-Cleavable Cleavage Site

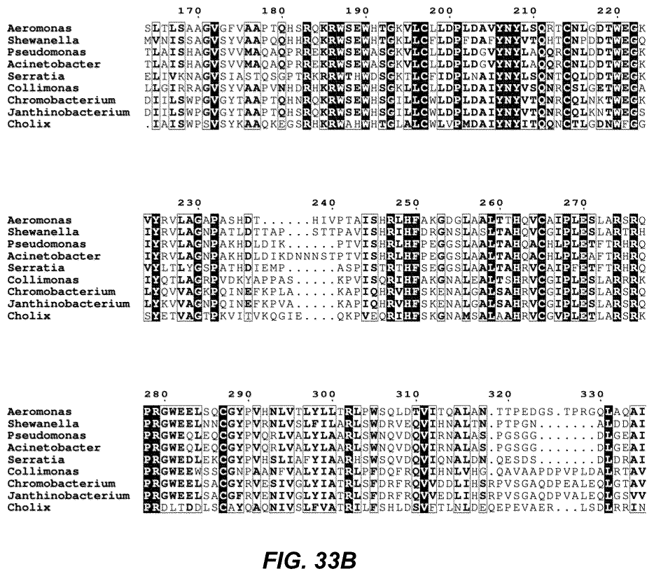

Some naturally occurring mARTs include one or more putative furin cleavage sites in one or more domains of the mART. Some naturally occurring putative furin cleavage sites that occur within a domain I of mART are resistant to cleavage by a furin protease. Without being bound by theory, it is believed that some putative furin cleavage sites within a domain I of a mART are not efficiently cleaved by a furin protease due to the presence of a tryptophan residue that is positioned immediately C-terminal of the putative scissile bond (see FIGS. 33A and 33B), showing a W positioned immediately C-terminal of a RHKR sequence for cholix, along with other sequences showing conservation of this tryptophan residue). This application discloses various changes (e.g., mutations, insertions, deletions) to a mART sequence that can endow a delivery construct that includes a transcytosing element from a domain I of a mART with increased susceptibility to a furin protease, thereby facilitating release of a cargo from delivery construct. Stated differently, the disclosure herein provides delivery constructs that include a furin-cleavable cleavage site that can release a cargo from an N-terminal fragment that includes a transcytosing element.

The delivery constructs disclosed herein may include a furin-cleavable cleavage site. The furin-cleavable cleavage site may have a scissile bond that is positioned between the transcytosing element and the heterologous cargo such that cleavage of the scissile bond by a furin protease (e.g., a furin protease that the construct encounters upon being delivered to the basolateral side of an epithelial membrane) releases the heterologous cargo from the transcytosing element. The scissile bond of the furin-cleavable cleavage site may be positioned such that, upon cleavage of the scissile bond by the furin protease, a released N-terminal fragment comprising the transcytosing element lacks (1) a C-terminal portion of domain I of a mART, (2) a domain II of a mART, and (3) a domain III of a mART.

In some embodiments, the furin-cleavable cleavage site comprises a contiguous sequence of P4-P3-P2-P1-P1′-P2′-P3′-P4′, wherein P1 is Arg, wherein P2, P3, P4, P1′, P2′, P3′, and P4′ are each independently selected from any naturally occurring amino acid, and wherein the scissile bond of the cleavage site is positioned between P1-P1′.

In some embodiments, P2 is Arg or Lys and P4 is Arg. In some embodiments, P3 is selected from the group consisting of histidine, glutamine, lysine, and glutamic acid. In some embodiments, P2 is Lys and P3 is His. In some embodiments, P3 is not Thr. In some embodiments, P1′ is not a tryptophan. In some embodiments, P1′ is Ser. In some embodiments, P1′ is Gly. In some embodiments, P1′ is His. In some embodiments, P2′ is selected from the group consisting of Val, Ala, Leu, Ile, Ser, Thr, and Lys. In some embodiments, P2′ is Gly. In some embodiments, P3′ is selected from the group consisting of Gly, Ala, Leu, Ser, Thr, Asp, Glu, and Arg. In some embodiments, P4′ is selected from the group consisting of Gly, Pro, Ala, Leu, Ser, Thr, Asp, Glu, Lys, and Arg. In some embodiments, P1′ is Ser, P2′ is Ala, P3′ is Ala, and P4′ is Gly. In some embodiments, P1′ is His, P2′ is Gly, P3′ is Glu, and P4′ is Gly.

In some embodiments, the furin-cleavable cleavage site further comprises a contiguous sequence of P14-P13-P12-P11-P10-P9-P8-P7-P6-P5 positioned immediately N-terminal of P4, wherein P14 is Ser or Gly and wherein P9 is Gln or Pro. In some embodiments, the furin-cleavable cleavage site further comprises a contiguous sequence of P14-P13-P12-P11-P10-P9-P8-P7-P6-P5 positioned immediately N-terminal of P4, wherein P14 is Ser and wherein P9 is Gln. In some embodiments, the contiguous sequence of P14-P13-P12-P11-P10-P9-P8-P7-P6-P5 comprises SYKAAQKEGS. In some embodiments, the contiguous sequence of P14-P13-P12-P11-P10-P9-P8-P7-P6-P5 comprises SVVMAQAQPR.

Delivery constructs described herein may be cleaved by a furin protease. For instance, for some delivery constructs, at least 50% of the delivery construct is cleaved by furin after 18 h at room temperature under the following conditions: 2.5 μg of delivery construct, 2 μg of furin, 20 mM HEPES at pH 5, 0.2 mM CaCl₂), 0.2 mM TCEP in a total of between 25 and 30 microliters. In some embodiments, at least 5%, 10%, 20%, 30%, 40%, 50%, 60%, 70%, 80%, 90% or 95% of the delivery construct is cleaved at a scissile bond within the cleavage site under such conditions.

In some embodiments, the scissile bond is positioned between amino acid residues 186 and 260 of SEQ ID NO: 43 when the transcytosing element and the furin-cleavable cleavage site are optimally aligned with SEQ ID NO: 43. In some embodiments, the scissile bond is positioned between amino acid residues 186 and 248 of SEQ ID NO: 44 when the transcytosing element and the furin-cleavable cleavage site are optimally aligned with SEQ ID NO: 44.

In some embodiments, the furin-cleavable delivery construct colocalizes with furin in an endosome during transcytosis, but is not cleaved by furin within the endosome. Rather, the delivery construct may instead be cleaved by furin upon exit from the polarized epithelial cell.

In some embodiments, when the transcytosing element is optimally aligned with the domain I of the mART from which it is derived, P1, P2, P3, and P4 are each identical to the amino acid to which they are aligned. In some embodiments, when the delivery construct is optimally aligned with SEQ ID NO: 43, the scissile bond of the delivery construct is C-terminal of an amino acid that is aligned with the Lys at position 186 of SEQ ID NO: 43. In some embodiments, when delivery construct is optimally aligned with SEQ ID NO: 43, the scissile bond of the delivery construct is N-terminal of an amino acid that is aligned with the Arg at position 260 of SEQ ID NO: 43. In some embodiments, when delivery construct is optimally aligned with SEQ ID NO: 43, the scissile bond of the delivery construct is N-terminal of an amino acid that is aligned with the Ile at position 261 of SEQ ID NO: 43. In some embodiments, when delivery construct is optimally aligned with SEQ ID NO: 44, the scissile bond of the delivery construct is C-terminal of an amino acid that is aligned with the Arg at position 186 of SEQ ID NO: 44. In some embodiments, when the delivery construct is optimally aligned with SEQ ID NO: 44, the scissile bond of the delivery construct is N-terminal of an amino acid that is aligned with the Leu at position 248 of SEQ ID NO: 44.

In some embodiments, the scissile bond is positioned C-terminal of a core 12-14-stranded β-jellyroll fold of the transcytosing element. In some embodiments, the transcytosing element comprises or consists of SEQ ID NO: 20 (and optionally an N-terminal methionine), and the scissile bond is immediately C-terminal of position 197. Upon cleavage by a furin protease, the released N-terminal fragment may comprise or consist of SEQ ID NO: 20 (and optionally and N-terminal methionine). In some embodiments, the transcytosing element comprises or consists of SEQ ID NO: 20 with the VEDE sequence modified to VEEA (SEQ ID NO: 60) or LEEA (SEQ ID NO: 61) (and optionally an N-terminal methionine), and the scissile bond is immediately C-terminal of position 197. Upon cleavage by a furin protease, the released N-terminal fragment may comprise or consist of SEQ ID NO: 20 with the VEDE sequence modified to VEEA (SEQ ID NO: 60) or LEEA (SEQ ID NO: 61) (and optionally and N-terminal methionine). In some embodiments, the transcytosing element comprises or consists of SEQ ID NO: 52 (and optionally an N-terminal methionine), and the scissile bond is immediately C-terminal of position 186. Upon cleavage by a furin protease, the released N-terminal fragment may comprise or consist of SEQ ID NO: 52.

### VII. Coupling of the Payload to a Transcytosing Element

In some cases, the compositions provided herein comprise a transcytosing element coupled to a payload, e.g., a heterologous payload. In some cases, one or more payloads are fused to a carrier, resulting in a fusion molecule. In various embodiments, the payload is directly coupled to C-terminus of the carrier. In some embodiments, the payload is indirectly coupled to the carrier via a spacer or linker.

In embodiments where the payload is expressed together with another sequence as a fusion protein, the payload can be inserted into the fusion molecule by any method known to one of skill in the art without limitation. For example, nucleic acids coding for amino acids corresponding to the payload can be directly inserted into the nucleic acid coding for the other moiety or fusion molecule, with or without deletion of native amino acid sequences.

### VIII. Production of Nucleic Acids Encoding Carriers and/or Payloads

In various embodiments, the transcytosing elements (carriers), payloads, and/or delivery constructs are prepared using the methodology described in, e.g., U.S. Pat. Nos. 9,090,691 and 7,713,737, each incorporated by reference herein in their entirety.

In various embodiments, the transcytosing elements (carriers), payloads, and/or non-naturally occurring fusion molecules are synthesized using recombinant DNA methodology. Generally, this can involve creating a DNA sequence that encodes the transcytosing element (carrier), furin cleavage sequence, payload, and/or fusion molecule, placing the DNA in an expression cassette under the control of a particular promoter, expressing the molecule in a host, isolating the expressed molecule and, if required, folding of the molecule into an active conformational form.

DNA encoding the carrier, payload, and/or fusion molecules described herein can be prepared by any suitable method, including, for example, cloning and restriction of appropriate sequences or direct chemical synthesis by methods such as the phosphotriester method of Narang et al. (1979) Meth. Enzymol. 68:90-99; the phosphodiester method of Brown et al. (1979) Meth. Enzymol. 68:109-151; the diethylphosphoramidite method of Beaucage et al. (1981) Tetra. Lett., 22:1859-1862); the solid support method of U.S. Pat. No. 4,458,066, and the like.

Chemical synthesis can produce a single stranded oligonucleotide. This can be converted into double stranded DNA by hybridization with a complementary sequence or by polymerization with a DNA polymerase using the single strand as a template. Chemical synthesis can be used to generated DNA sequences of about 100 bases. Longer sequences can be obtained by the ligation of shorter sequences.

Alternatively, sub-sequences can be cloned and the appropriate sub-sequences cleaved using appropriate restriction enzymes. The fragments can then be ligated to produce the desired DNA sequence.

In various embodiments, DNA encoding carrier, payload, and/or fusion molecules of the present disclosure can be cloned using DNA amplification methods such as polymerase chain reaction (PCR). Thus, for example, the gene or genes for the one or more payloads, e.g. the one or more biologically-active payloads is PCR amplified using sense and anti-sense primers having N-terminal restriction sites. This can produce one or more nucleic acids encoding the one or more payload sequences and having terminal restriction sites. A carrier having “complementary” restriction sites can similarly be cloned and then ligated to the one or more nucleic acids encoding the one or more payloads and/or to a linker attached to the one or more nucleic acids encoding the one or more payloads. Ligation of the nucleic acid sequences and insertion into a vector produces a vector encoding the one or more payloads joined to the carrier/s.

### IX. Polynucleotides Encoding Carriers, Payloads, and Fusion Molecules

In another aspect, the disclosure provides polynucleotides comprising a nucleotide sequence encoding a transcytosing element (carrier), a payload (e.g., a heterologous payload), and fusion molecules (e.g., delivery constructs). These polynucleotides are useful, for example, for making a transcytosing element (carrier), a payload (e.g., a heterologous payload), and fusion molecules (e.g., delivery constructs). In yet another aspect, the disclosure provides an expression system that comprises a recombinant polynucleotide sequence encoding a transcytosing element, a furin-cleavable cleavage site, and a polylinker insertion site for a polynucleotide sequence encoding a payload. In some embodiments, the polynucleotide further includes a regulatory sequence operably linked to the polynucleotide sequence.

Various in vitro methods that can be used to prepare a polynucleotide encoding a transcytosing element (e.g., a mART-derived carrier sequence), payload, or fusion molecules of the disclosure include, but are not limited to, reverse transcription, the polymerase chain reaction (PCR), the ligase chain reaction (LCR), the transcription-based amplification system (TAS), the self-sustained sequence replication system (3SR) and the QP replicase amplification system (QB).

Guidance for using these cloning and in vitro amplification methodologies are described in, for example, U.S. Pat. No. 4,683,195; Mullis et al., 1987, Cold Spring Harbor Symp. Quant. Biol. 51:263; and Erlich, ed., 1989, PCR Technology, Stockton Press, NY. Polynucleotides encoding a fusion molecule or a portion thereof also can be isolated by screening genomic or cDNA libraries with probes selected from the sequences of the desired polynucleotide under stringent, moderately stringent, or highly stringent hybridization conditions.

Construction of nucleic acids encoding the carriers, payloads, or fusion molecules of the disclosure can be facilitated by introducing an insertion site for a nucleic acid into the construct.

Further, the polynucleotides can also encode a secretory sequence at the amino terminus of the encoded transcytosing element (carrier), payload, or fusion molecule (e.g., delivery construct). Such constructs are useful for producing the carriers, payloads, or fusion molecules in mammalian cells as they simplify isolation of the immunogen.

Furthermore, the polynucleotides of the disclosure also encompass derivative versions of polynucleotides encoding a transcytosing element (carrier), payload, or fusion molecule (e.g., delivery construct). For example, derivatives can be made by site-specific mutagenesis, including substitution, insertion, or deletion of one, two, three, five, ten or more nucleotides, of polynucleotides encoding the fusion molecule. Alternatively, derivatives can be made by random mutagenesis.

Accordingly, in various embodiments, the disclosure provides a polynucleotide that encodes a transcytosing element (carrier), payload, or fusion molecule (e.g., delivery construct). The polynucleotide sequence may further encode for furin-cleavable cleavage site.

In various embodiments, the polynucleotide hybridizes under stringent hybridization conditions to any polynucleotide of this disclosure. In further embodiments, the polynucleotide hybridizes under stringent conditions to a nucleic acid that encodes any carrier, payload, or fusion molecule of the disclosure.

In still another aspect, the disclosure provides expression vectors for expressing the transcytosing elements (carriers), payloads, or fusion molecules. Generally, expression vectors can be recombinant polynucleotide molecules comprising expression control sequences operatively linked to a nucleotide sequence encoding a polypeptide. Expression vectors can readily be adapted for function in prokaryotes or eukaryotes by inclusion of appropriate promoters, replication sequences, selectable markers, etc. to result in stable transcription and translation or mRNA. Techniques for construction of expression vectors and expression of genes in cells comprising the expression vectors are well known in the art. See, e.g., Sambrook et al., 2001, Molecular Cloning—A Laboratory Manual, 3.sup.rd edition, Cold Spring Harbor Laboratory, Cold Spring Harbor, N.Y., and Ausubel et al., eds., Current Edition, Current Protocols in Molecular Biology, Greene Publishing Associates and Wiley Interscience, NY.

The expression vectors can contain expression and replication signals compatible with the cell in which the carriers, payloads, or fusion molecules are expressed. The expression vectors can be introduced into the cell for expression of the carriers, payloads, or fusion molecules by any method known to one of skill in the art without limitation. The expression vectors can also contain a purification moiety that simplifies isolation of the carrier, payload, or fusion molecule.

In yet another aspect, the disclosure provides a cell comprising an expression vector for expression of the carriers, payloads, or fusion molecules, or portions thereof. The cell can be selected for its ability to express high concentrations of the carrier, payload, or fusion molecule to facilitate purification of the protein. In various embodiments, the cell is a prokaryotic cell, for example, E. coli. As described, e.g., in the examples, the carriers, payloads, and fusion molecules can be properly folded and can comprise the appropriate disulfide linkages when expressed in E. coli.

In other embodiments, the cell is a eukaryotic cell. Useful eukaryotic cells include yeast and mammalian cells. Any mammalian cell known by one of skill in the art to be useful for expressing a recombinant polypeptide, without limitation, can be used to express the carriers, payloads, or fusion molecules. For example, Chinese hamster ovary (CHO) cells can be used to express the carriers, payloads, or fusion molecules.

The carrier, payloads, or fusion molecules of the disclosure can be produced by recombination, as described below. However, the carrier, payloads, or fusion molecules can also be produced by chemical synthesis using methods known to those of skill in the art.

Methods for expressing and purifying the transcytosing elements (carriers), payloads, and fusion molecules (delivery constructs) of the disclosure are described extensively herein, e.g., in the examples below. Generally, the methods can rely on introduction of an expression vector encoding the carrier, payload, and/or fusion molecule to a cell that can express the transcytosing element (carrier), payload, and/or fusion molecule (e.g., delivery construct) from the vector. The transcytosing element (carrier), payload, and/or fusion molecule can then be purified for administration to one or more epithelial cells.

### X. Experimental Methods

Methods are provided herein for measuring transcytosis, identifying interaction partners for targeting signals within carrier proteins, and for observing the intracellular localization of delivery constructs and other intracellular proteins that may participate in the transcytosis of delivery constructs.

A delivery construct may pass through layer of epithelial cells by transcytosis. During transcytosis, the delivery construct may interact with a cell surface protein displayed on the apical (external) surface of the cell. The delivery construct is internalized into the epithelial cell by endocytosis, transported from the apical domain of the cell to the basal domain of the cell, and then released by exocytosis across the basal plasma membrane. A delivery construct that is transported across an epithelial cell by transcytosis must avoid being targeted to the lysosome, where it would be degraded, or being recycled back to the apical surface.

Transcytosis of a delivery construct can be tested by any method known by one of skill in the art, without limitation. In cases of a mART-derived transcytosing element, and without intending to be bound to any particular theory or mechanism of action, it is described herein that the transcytosis function that allows a delivery construct to pass through a polarized epithelial cell and the function to enter non-polarized cells can reside in the same domain or region, i.e., Domain I.

The delivery construct's transcytosis ability can be tested by assessing a delivery construct's ability to pass through a polarized epithelial cell. For example, the delivery construct can be labeled with, for example, a fluorescent marker (e.g., RFP) and contacted to the apical membranes of a layer of epithelial cells. In another example, the delivery construct can be detected using antibodies (e.g., monoclonal and/or polyclonal antibodies) directed against the delivery construct, or a portion thereof such as a mART-derived carrier or a payload.

Fluorescence detected on the basolateral side of the membrane formed by the epithelial cells (e.g., a basolateral chamber or the lamina propria in in vivo experiments) indicates that the transcytosis capabilities of the carrier are intact.

Transcytosis can be tested in vitro using a transwell chamber. Epithelial cells are grown on the upper surface of a permeable membrane. Once the cells have grown to confluence, formation of an epithelial barrier can be detected by measuring electrical resistance. Apical to Basal transcytosis (vectorial transport) can be measured by adding a delivery construct to the apical chamber and observing its appearance over time in the basolateral chamber (and vice-versa for Basal to Apical transcytosis).

In vivo transcytosis can be tested using male Wistar rats. Male Wistar rats can be housed 3-5 per cage with a 12/12 h light/dark cycle and can be 225-275 g (approximately 6-8 weeks old) when placed on study. Experiments can be conducted during the light phase using a non-recovery protocol that uses continuous isoflurane anesthesia. A 4-5 cm midline abdominal incision that exposes mid-jejunum regions can be conducted. Stock solutions at 3.86×10⁻⁵ M of test articles can be prepared in phosphate buffered saline (PBS), and 50 μL (per 250 g rat) can be administered by intraluminal injection (ILI) using a 29-gauge needle. The injection site mesentery can then be marked with a permanent marker. At study termination, a 3-5 mm region that captured the marked intestine segment can be isolated and processed for microscopic assessment.

### XI. Methods of Use

Provided herein, in some embodiments, are delivery constructs comprising a transcytosing element coupled to a heterologous payload, wherein the delivery construct comprises furin-cleavable cleavage site as described herein. The transcytosing elements provided herein can be used to transport a payload (e.g., a therapeutic payload) to the basal side of an epithelial cell. Delivery across a polarized gut or respiratory epithelium can include delivery to submucosal compartments (e.g., lamina propria and/or other submucosal intestinal compartments) and/or systemic circulation (e.g., via the hepatic portal system).

The furin-cleavable delivery constructs described herein can be delivered to an apical side of an epithelial membrane such that the cargo (1) transcytoses across an epithelial cell of the epithelial cell of the epithelial membrane and (2) is cleaved from the transcytosing element by a furin protease to deliver the cargo to a basolateral side of the epithelial membrane. The epithelial cell may be any suitable epithelial cell, such as a gut epithelial cell or a respiratory epithelial cell.

The high flux transport capacities of carriers provided herein across intact epithelial barriers (e.g., a polarized gut epithelium) can be used to deliver therapeutic payload molecules to a subject in need thereof (e.g., a human or a rodent). For example, delivery of therapeutic payload to submucosal compartments, e.g., the lamina propria, can allow for delivery of the cargo to a location in the GI tract, whereas systemic delivery of payload can be used to provide desired concentration of the payload in various cell(s), tissue(s), or organ(s) within an organism.

A delivery construct can be administered via various administration routes. In some cases, administration includes oral administration of the delivery construct. In some instances, a delivery construct is orally administered as a tablet or a capsule.

TABLE 1
Sequences

| SEQ | | |
|---|---|---|
| ID NO | Description | Sequence |
| 1 | Non-naturally | VEEALNIFDECRSPCSLTPEPGKPIQSKLSIPSDVVLDEGVLYY |
| | occurring | SMTINDEQNDIKDEDKGESIITIGEFATVRATRHYVNQDAPFG |
| | cholix peptide | VIHLDITTENGTKTYSYNRKEGEFAINWLVPIGEDSPASIKISV |
| | (valine at | DELDQQRNIIEVPKLYSIDLDNQTLEQWKTQGNVSFSVTRPEH |
| | position 1) | NIAISWPSVSYKAAQKEGSRHKRWAHWHTGLALCWLVPMD |
| | | AIYNYITQQNCTLGDNWFGGSYETVAGTPKVITVKQGIEQKP |
| | | VEQRIHFSKGNAMSALAAHRVCGVPLETLARSRKPRDLTDDL |
| | | SCAYQAQNIVSLFVATRILFSHLDSVFTLNLDEQEPEVAERLS |
| | | DLRRINENNPGMVTQVLTVARQIYNDYVTHHPGLTPEQTSAG |
| | | AQAADILSLFCPDADKSCVASNNDQANINIESRSGRSYLPENR |
| | | AVITPQGVTNWTYQELEATHQALTREGYVFVGYHGTNHVAA |
| | | QTIVNRIAPVPRGNNTENEEKWGGLYVATHAEVAHGYARIKE |
| | | GTGEYGLPTRAERDARGVMLRVYIPRASLERFYRTNTPLENA |
| | | EEHITQVIGHSLPLRNEAFTGPESAGGEDETVIGWDMAIHAVA |
| | | IPSTIPGNAYEELAIDEEAVAKEQSISTKPPYKERKDELK |
| 2 | Non-naturally | LEEALNIFDECRSPCSLTPEPGKPIQSKLSIPSDVVLDEGVLYYS |
| | occurring | MTINDEQNDIKDEDKGESIITIGEFATVRATRHYVNQDAPFGV |
| | cholix peptide | IHLDITTENGTKTYSYNRKEGEFAINWLVPIGEDSP ASIKISVD |
| | (leucine at | ELDQQRNIIEVPKLYSIDLDNQTLEQWKTQGNVSFSVTRPEHN |
| | position 1) | IAISWPSVSYKAAQKEGSRHKRWAHWHTGLALCWLVPMDAI |
| | | YNYITQQNCTLGDNWFGGSYETVAGTPKVITVKQGIEQKPVE |
| | | QRIHFSKGNAMSALAAHRVCGVPLETLARSRKPRDLTDDLSC |
| | | AYQAQNIVSLFVATRILFSHLDSVFTLNLDEQEPEVAERLSDL |
| | | RRINENNPGMVTQVLTVARQIYNDYVTHHPGLTPEQTSAGAQ |
| | | AADILSLFCPDADKSCVASNNDQANINIESRSGRSYLPENRAVI |
| | | TPQGVTNWTYQELEATHQALTREGYVFVGYHGTNHVAAQTI |
| | | VNRIAPVPRGNNTENEEKWGGLYVATHAEVAHGYARIKEGT |
| | | GEYGLPTRAERDARGVMLRVYIPRASLERFYRTNTPLENAEE |
| | | HITQVIGHSLPLRNEAFTGPESAGGEDETVIGWDMAIHAVAIP |
| | | STIPGNAYEELAIDEEAVAKEQSISTKPPYKERKDELK |
| 3 | Naturally | VEDELNIFDECRSPCSLTPEPGKPIQSKLSIPSDVVLDEGVLYY |
| | occurring | SMTINDEQNDIKDEDKGESIITIGEFATVRATRHYVNQDAPFG |
| | cholix peptide | VIHLDITTENGTKTYSYNRKEGEFAINWLVPIGEDSPASIKISV |
| | | DELDQQRNIIEVPKLYSIDLDNQTLEQWKTQGNVSFSVTRPEH |
| | | NIAISWPSVSYKAAQKEGSRHKRWAHWHTGLALCWLVPMD |
| | | AIYNYITQQNCTLGDNWFGGSYETVAGTPKVITVKQGIEQKP |
| | | VEQRIHFSKGNAMSALAAHRVCGVPLETLARSRKPRDLTDDL |
| | | SCAYQAQNIVSLFVATRILFSHLDSVFTLNLDEQEPEVAERLS |
| | | DLRRINENNPGMVTQVLTVARQIYNDYVTHHPGLTPEQTSAG |
| | | AQAADILSLFCPDADKSCVASNNDQANINIESRSGRSYLPENR |
| | | AVITPQGVTNWTYQELEATHQALTREGYVFVGYHGTNHVAA |
| | | QTIVNRIAPVPRGNNTENEEKWGGLYVATHAEVAHGYARIKE |
| | | GTGEYGLPTRAERDARGVMLRVYIPRASLERFYRTNTPLENA |
| | | EEHITQVIGHSLPLRNEAFTGPESAGGEDETVIGWDMAIHAVA |
| | | IPSTIPGNAYEELAIDEEAVAKEQSISTKPPYKERKDELK |
| 4 | M + cholix²⁶⁶ | MVEEALNIFDECRSPCSLTPEPGKPIQSKLSIPSDVVLDEGVLY |
| | (valine at | YSMTINDEQNDIKDEDKGESIITIGEFATVRATRHYVNQDAPF |
| | position 1) | GVIHLDITTENGTKTYSYNRKEGEFAINWLVPIGEDSPASIKIS |
| | | VDELDQQRNIIEVPKLYSIDLDNQTLEQWKTQGNVSFSVTRPE |
| | | HNIAISWPSVSYKAAQKEGSRHKRWAHWHTGLALCWLVPM |
| | | DAIYNYITQQNCTLGDNWFGGSYETVAGTPKVITVKQGIEQK |
| | | PVEQRIHFSKG |
| 5 | Cholix²⁶⁶ | VEEALNIFDECRSPCSLTPEPGKPIQSKLSIPSDVVLDEGVLYY |
| | (valine at | SMTINDEQNDIKDEDKGESIITIGEFATVRATRHYVNQDAPFG |
| | position 1) | VIHLDITTENGTKTYSYNRKEGEFAINWLVPIGEDSPASIKISV |
| | | DELDQQRNIIEVPKLYSIDLDNQTLEQWKTQGNVSFSVTRPEH |
| | | NIAISWPSVSYKAAQKEGSRHKRWAHWHTGLALCWLVPMD |
| | | AIYNYITQQNCTLGDNWFGGSYETVAGTPKVITVKQGIEQKP |
| | | VEQRIHFSKG |
| 6 | cholix¹⁻²⁶⁶ | LEEALNIFDECRSPCSLTPEPGKPIQSKLSIPSDVVLDEGVLYYS |
| | carrier | MTINDEQNDIKDEDKGESIITIGEFATVRATRHYVNQDAPFGV |
| | (leucine at | IHLDITTENGTKTYSYNRKEGEFAINWLVPIGEDSPASIKISVD |
| | position 1) | ELDQQRNIIEVPKLYSIDLDNQTLEQWKTQGNVSFSVTRPEHN |
| | | IAISWPSVSYKAAQKEGSRHKRWAHWHTGLALCWLVPMDAI |
| | | YNYITQQNCTLGDNWFGGSYETVAGTPKVITVKQGIEQKPVE |
| | | QRIHFSKG |
| 7 | M + His₆_Chx¹⁹⁷_ | MHHHHHHVEDELNIFDECRSPCSLTPEPGKPIQSKLSIPSDVVL |
| | hGH | DEGVLYYSMTINDEQNDIKDEDKGESIITIGEFATVRATRHYV |
| | | QQDAPFGVIHLDITTEQGTKTYSYNRKEGEFAINWLVPIGEDS |
| | | PASIKISVDELDQQRNIIEVPKLYSIDLDQQTLEQWKTQGQVSF |
| | | SVTRPEHNIAISWPSVSYKAAQKEGSRHKRFPTIPLSRLFDNA |
| | | MLRAHRLHQLAFDTYQEFEEAYIPKEQKYSFLQNPQTSLCFSE |
| | | SIPTPSNREETQQKSNLELLRISLLLIQSWLEPVQFLRSVFANSL |
| | | VYGASDSNVYDLLKDLEEGIQTLMGRLEDGSPRTGQIFKQTY |
| | | SKFDTNSHNDDALLKNYGLLYCFRKDMDKVETFLRIVQCRS |
| | | VEGSCGF |
| 8 | M + His₆_Chx¹⁹⁷_ | MHHHHHHVEDELNIFDECRSPCSLTPEPGKPIQSKLSIPSDVVL |
| | G_hGH | DEGVLYYSMTINDEQNDIKDEDKGESIITIGEFATVRATRHYV |
| | | QQDAPFGVIHLDITTEQGTKTYSYNRKEGEFAINWLVPIGEDS |
| | | PASIKISVDELDQQRNIIEVPKLYSIDLDQQTLEQWKTQGQVSF |
| | | SVTRPEHNIAISWPSVSYKAAQKEGSRHKRGFPTIPLSRLFDN |
| | | AMLRAHRLHQLAFDTYQEFEEAYIPKEQKYSFLQNPQTSLCF |
| | | SESIPTPSNREETQQKSNLELLRISLLLIQSWLEPVQFLRSVFAN |
| | | SLVYGASDSNVYDLLKDLEEGIQTLMGRLEDGSPRTGQIFKQ |
| | | TYSKFDTNSHNDDALLKNYGLLYCFRKDMDKVETFLRIVQC |
| | | RSVEGSCGF |
| 9 | M + His₆_Chx¹⁹⁷_ | MHHHHHHVEDELNIFDECRSPCSLTPEPGKPIQSKLSIPSDVVL |
| | GG_hGH | DEGVLYYSMTINDEQ |
| | | NDIKDEDKGESIITIGEFATVRATRHYVQQDAPFGVIHLDITTE |
| | | QGTKTYSYNRKEGEFAINWLVPIGEDSPASIKISVDELDQQRNI |
| | | IEVPKLYSIDLDQQTLEQWKTQGQVSFSVTRPEHNIAISWPSV |
| | | SYKAAQKEGSRHKRGGFPTIPLSRLFDNAMLRAHRLHQLAFD |
| | | TYQEFEEAYIPKEQKYSFLQNPQTSLCFSESIPTPSNREETQQK |
| | | SNLELLRISLLLIQSWLEPVQFLRSVFANSLVYGASDSNVYDL |
| | | LKDLEEGIQTLMGRLEDGSPRTGQIFKQTYSKFDTNSHNDDA |
| | | LLKNYGLLYCFRKDMDKVETFLRIVQCRSVEGSCGF |
| 10 | M + His₆_Chx¹⁹⁷_ | MHHHHHHVEDELNIFDECRSPCSLTPEPGKPIQSKLSIPSDVVL |
| | GGG_hGH | DEGVLYYSMTINDEQNDIKDEDKGESIITIGEFATVRATRHYV |
| | | QQDAPFGVIHLDITTEQGTKTYSYNRKEGEFAINWLVPIGEDS |
| | | PASIKISVDELDQQRNIIEVPKLYSIDLDQQTLEQWKTQGQVSF |
| | | SVTRPEHNIAISWPSVSYKAAQKEGSRHKRGGGFPTIPLSRLF |
| | | DNAMLRAHRLHQLAFDTYQEFEEAYIPKEQKYSFLQNPQTSL |
| | | CFSESIPTPSNREETQQKSNLELLRISLLLIQSWLEPVQFLRSVF |
| | | ANSLVYGASDSNVYDLLKDLEEGIQTLMGRLEDGSPRTGQIF |
| | | KQTYSKFDTNSHNDDALLKNYGLLYCFRKDMDKVETFLRIV |
| | | QCRSVEGSCGF |
| 11 | M + His₆_Chx¹⁹⁷_ | MHHHHHHVEDELNIFDECRSPCSLTPEPGKPIQSKLSIPSDVVL |
| | SAAG _hGH | DEGVLYYSMTINDEQNDIKDEDKGESIITIGEFATVRATRHYV |
| | | NQDAPFGVIHLDITTENGTKTYSYNRKEGEFAINWLVPIGEDS |
| | | PASIKISVDELDQQRNIIEVPKLYSIDLDNQTLEQWKTQGNVSF |
| | | SVTRPEHNIAISWPSVSYKAAQKEGSRHKRSAAGFPTIPLSRLF |
| | | DNAMLRAHRLHQLAFDTYQEFEEAYIPKEQKYSFLQNPQTSL |
| | | CFSESIPTPSNREETQQKSNLELLRISLLLIQSWLEPVQFLRSVF |
| | | ANSLVYGASDSNVYDLLKDLEEGIQTLMGRLEDGSPRTGQIF |
| | | KQTYSKFDTNSHNDDALLKNYGLLYCFRKDMDKVETFLRIV |
| | | QCRSVEGSCGF |
| 12 | M + His₆_Chx¹⁹⁷_ | MHHHHHHVEDELNIFDECRSPCSLTPEPGKPIQSKLSIPSDVVL |
| | SAAG_ | DEGVLYYSMTINDEQNDIKDEDKGESIITIGEFATVRATRHYV |
| | G₃SG₃SG₃S_ | NQDAPFGVIHLDITTENGTKTYSYNRKEGEFAINWLVPIGEDS |
| | hGH | PASIKISVDELDQQRNIIEVPKLYSIDLDNQTLEQWKTQGNVSF |
| | | SVTRPEHNIAISWPSVSYKAAQKEGSRHKRSAAGGGGSGGGS |
| | | GGGSFPTIPLSRLFDNAMLRAHRLHQLAFDTYQEFEEAYIPKE |
| | | QKYSFLQNPQTSLCFSESIPTPSNREETQQKSNLELLRISLLLIQ |
| | | SWLEPVQFLRSVFANSLVYGASDSNVYDLLKDLEEGIQTLMG |
| | | RLEDGSPRTGQIFKQTYSKFDTNSHNDDALLKNYGLLYCFRK |
| | | DMDKVETFLRIVQCRSVEGSCGF |
| 13 | M + His₆_Chx¹⁹⁷_ | MHHHHHHVEDELNIFDECRSPCSLTPEPGKPIQSKLSIPSDVVL |
| | ERTKR_ | DEGVLYYSMTINDEQNDIKDEDKGESIITIGEFATVRATRHYV |
| | G₃SG₃SG₃S_ | NQDAPFGVIHLDITTENGTKTYSYNRKEGEFAINWLVPIGEDS |
| | hGH | PASIKISVDELDQQRNIIEVPKLYSIDLDNQTLEQWKTQGNVSF |
| | | SVTRPEHNIAISWPSVSYKAAQKEGSERTKRGGGSGGGSGGG |
| | | SFPTIPLSRLFDNAMLRAHRLHQLAFDTYQEFEEAYIPKEQKY |
| | | SFLQNPQTSLCFSESIPTPSNREETQQKSNLELLRISLLLIQSWL |
| | | EPVQFLRSVFANSLVYGASDSNVYDLLKDLEEGIQTLMGRLE |
| | | DGSPRTGQIFKQTYSKFDTNSHNDDALLKNYGLLYCFRKDM |
| | | DKVETFLRIVQCRSVEGSCGF |
| 14 | Chx¹⁹⁷_hGH | VEDELNIFDECRSPCSLTPEPGKPIQSKLSIPSDVVLDEGVLYY |
| | | SMTINDEQNDIKDEDKGESIITIGEFATVRATRHYVQQDAPFG |
| | | VIHLDITTEQGTKTYSYNRKEGEFAINWLVPIGEDSPASIKISV |
| | | DELDQQRNIIEVPKLYSIDLDQQTLEQWKTQGQVSFSVTRPEH |
| | | NIAISWPSVSYKAAQKEGSRHKRFPTIPLSRLFDNAMLRAHRL |
| | | HQLAFDTYQEFEEAYIPKEQKYSFLQNPQTSLCFSESIPTPSNR |
| | | EETQQKSNLELLRISLLLIQSWLEPVQFLRSVFANSLVYGASDS |
| | | NVYDLLKDLEEGIQTLMGRLEDGSPRTGQIFKQTYSKFDTNS |
| | | HNDDALLKNYGLLYCFRKDMDKVETFLRIVQCRSVEGSCGF |
| 15 | Chx¹⁹⁷_G_hGH | VEDELNIFDECRSPCSLTPEPGKPIQSKLSIPSDVVLDEGVLYY |
| | | SMTINDEQNDIKDEDKGESIITIGEFATVRATRHYVQQDAPFG |
| | | VIHLDITTEQGTKTYSYNRKEGEFAINWLVPIGEDSPASIKISV |
| | | DELDQQRNIIEVPKLYSIDLDQQTLEQWKTQGQVSFSVTRPEH |
| | | NIAISWPSVSYKAAQKEGSRHKRGFPTIPLSRLFDNAMLRAHR |
| | | LHQLAFDTYQEFEEAYIPKEQKYSFLQNPQTSLCFSESIPTPSN |
| | | REETQQKSNLELLRISLLLIQSWLEPVQFLRSVFANSLVYGAS |
| | | DSNVYDLLKDLEEGIQTLMGRLEDGSPRTGQIFKQTYSKFDT |
| | | NSHNDDALLKNYGLLYCFRKDMDKVETFLRIVQCRSVEGSC |
| | | GF |
| 16 | Chx¹⁹⁷_GG_hGH | VEDELNIFDECRSPCSLTPEPGKPIQSKLSIPSDVVLDEGVLYY |
| | | SMTINDEQNDIKDEDKGESIITIGEFATVRATRHYVQQDAPFG |
| | | VIHLDITTEQGTKTYSYNRKEGEFAINWLVPIGEDSPASIKISV |
| | | DELDQQRNIIEVPKLYSIDLDQQTLEQWKTQGQVSFSVTRPEH |
| | | NIAISWPSVSYKAAQKEGSRHKRGGFPTIPLSRLFDNAMLRAH |
| | | RLHQLAFDTYQEFEEAYIPKEQKYSFLQNPQTSLCFSESIPTPS |
| | | NREETQQKSNLELLRISLLLIQSWLEPVQFLRSVFANSLVYGA |
| | | SDSNVYDLLKDLEEGIQTLMGRLEDGSPRTGQIFKQTYSKFDT |
| | | NSHNDDALLKNYGLLYCFRKDMDKVETFLRIVQCRSVEGSC |
| | | GF |
| 17 | Chx¹⁹⁷_GGG_ | VEDELNIFDECRSPCSLTPEPGKPIQSKLSIPSDVVLDEGVLYY |
| | hGH | SMTINDEQNDIKDEDKGESIITIGEFATVRATRHYVQQDAPFG |
| | | VIHLDITTEQGTKTYSYNRKEGEFAINWLVPIGEDSPASIKISV |
| | | DELDQQRNIIEVPKLYSIDLDQQTLEQWKTQGQVSFSVTRPEH |
| | | NIAISWPSVSYKAAQKEGSRHKRGGGFPTIPLSRLFDNAMLRA |
| | | HRLHQLAFDTYQEFEEAYIPKEQKYSFLQNPQTSLCFSESIPTP |
| | | SNREETQQKSNLELLRISLLLIQSWLEPVQFLRSVFANSLVYG |
| | | ASDSNVYDLLKDLEEGIQTLMGRLEDGSPRTGQIFKQTYSKF |
| | | DTNSHNDDALLKNYGLLYCFRKDMDKVETFLRIVQCRSVEG |
| | | SCGF |
| 18 | Cholix¹⁹⁷_ | VEDELNIFDECRSPCSLTPEPGKPIQSKLSIPSDVVLDEGVLYY |
| | SAAG_hGH | SMTINDEQNDIKDEDKGESIITIGEFATVRATRHYVNQDAPFG |
| | | VIHLDITTENGTKTYSYNRKEGEFAINWLVPIGEDSPASIKISV |
| | | DELDQQRNIIEVPKLYSIDLDNQTLEQWKTQGNVSFSVTRPEH |
| | | NIAISWPSVSYKAAQKEGSRHKRSAAGFPTIPLSRLFDNAMLR |
| | | AHRLHQLAFDTYQEFEEAYIPKEQKYSFLQNPQTSLCFSESIPT |
| | | PSNREETQQKSNLELLRISLLLIQSWLEPVQFLRSVFANSLVYG |
| | | ASDSNVYDLLKDLEEGIQTLMGRLEDGSPRTGQIFKQTYSKF |
| | | DTNSHNDDALLKNYGLLYCFRKDMDKVETFLRIVQCRSVEG |
| | | SCGF |
| 19 | Chx¹⁹⁷_SAAG_ | VEDELNIFDECRSPCSLTPEPGKPIQSKLSIPSDVVLDEGVLYY |
| | G₃SG₃SG₃S | SMTINDEQNDIKDEDKGESIITIGEFATVRATRHYVNQDAPFG |
| | | VIHLDITTENGTKTYSYNRKEGEFAINWLVPIGEDSPASIKISV |
| | | DELDQQRNIIEVPKLYSIDLDNQTLEQWKTQGNVSFSVTRPEH |
| | | NIAISWPSVSYKAAQKEGSRHKRSAAGGGGSGGGSGGGSFPT |
| | | IPLSRLFDNAMLRAHRLHQLAFDTYQEFEEAYIPKEQKYSFLQ |
| | | NPQTSLCFSESIPTPSNREETQQKSNLELLRISLLLIQSWLEPVQ |
| | | FLRSVFANSLVYGASDSNVYDLLKDLEEGIQTLMGRLEDGSP |
| | | RTGQIFKQTYSKFDTNSHNDDALLKNYGLLYCFRKDMDKVE |
| | | TFLRIVQCRSVEGSCGF |
| 20 | Chx¹⁹⁷ | VEDELNIFDECRSPCSLTPEPGKPIQSKLSIPSDVVLDEGVLYY |
| | | SMTINDEQNDIKDEDKGESIITIGEFATVRATRHYVQQDAPFG |
| | | VIHLDITTEQGTKTYSYNRKEGEFAINWLVPIGEDSPASIKISV |
| | | DELDQQRNIIEVPKLYSIDLDQQTLEQWKTQGQVSFSVTRPEH |
| | | NIAISWPSVSYKAAQKEGSRHKR |
| 21 | Chx¹⁹⁷_G | VEDELNIFDECRSPCSLTPEPGKPIQSKLSIPSDVVLDEGVLYY |
| | | SMTINDEQNDIKDEDKGESIITIGEFATVRATRHYVQQDAPFG |
| | | VIHLDITTEQGTKTYSYNRKEGEFAINWLVPIGEDSPASIKISV |
| | | DELDQQRNIIEVPKLYSIDLDQQTLEQWKTQGQVSFSVTRPEH |
| | | NIAISWPSVSYKAAQKEGSRHKRG |
| 22 | Chx¹⁹⁷_GG | VEDELNIFDECRSPCSLTPEPGKPIQSKLSIPSDVVLDEGVLYY |
| | | SMTINDEQNDIKDEDKGESIITIGEFATVRATRHYVQQDAPFG |
| | | VIHLDITTEQGTKTYSYNRKEGEFAINWLVPIGEDSPASIKISV |
| | | DELDQQRNIIEVPKLYSIDLDQQTLEQWKTQGQVSFSVTRPEH |
| | | NIAISWPSVSYKAAQKEGSRHKRGG |
| 23 | Chx¹⁹⁷_GGG | VEDELNIFDECRSPCSLTPEPGKPIQSKLSIPSDVVLDEGVLYY |
| | | SMTINDEQNDIKDEDKGESIITIGEFATVRATRHYVQQDAPFG |
| | | VIHLDITTEQGTKTYSYNRKEGEFAINWLVPIGEDSPASIKISV |
| | | DELDQQRNIIEVPKLYSIDLDQQTLEQWKTQGQVSFSVTRPEH |
| | | NIAISWPSVSYKAAQKEGSRHKRGGG |
| 24 | Chx¹⁹⁷_SAAG | VEDELNIFDECRSPCSLTPEPGKPIQSKLSIPSDVVLDEGVLYY |
| | | SMTINDEQNDIKDEDKGESIITIGEFATVRATRHYVNQDAPFG |
| | | VIHLDITTENGTKTYSYNRKEGEFAINWLVPIGEDSPASIKISV |
| | | DELDQQRNIIEVPKLYSIDLDNQTLEQWKTQGNVSFSVTRPEH |
| | | NIAISWPSVSYKAAQKEGSRHKRSAAG |
| 25 | Chx¹⁹⁷_SAAG_ | VEDELNIFDECRSPCSLTPEPGKPIQSKLSIPSDVVLDEGVLYY |
| | G₃SG₃SG₃S | SMTINDEQNDIKDEDKGESIITIGEFATVRATRHYVNQDAPFG |
| | | VIHLDITTENGTKTYSYNRKEGEFAINWLVPIGEDSPASIKISV |
| | | DELDQQRNIIEVPKLYSIDLDNQTLEQWKTQGNVSFSVTRPEH |
| | | NIAISWPSVSYKAAQKEGSRHKRSAAGGGGSGGGSGGGS |
| 26 | G linker | G |
| 27 | GG linker | GG |
| 28 | GGG linker | GGG |
| 29 | SAAG linker | SAAG |
| 30 | SAAG- | SAAGGGGSGGGSGGGS |
| | G₃SG₃SG₃S | |
| | linker | |
| 31 | G₃SG₃SG₃S | GGGSGGGSGGGS |
| | linker | |
| 32 | G₄SG₄SG₄S | GGGGSGGGGSGGGGS |
| | linker | |
| 33 | F2 linker | GGGGGSNLQGGLRQPR |
| 34 | M + Chx²⁶⁶ + | MVEEALNIFDECRSPCSLTPEPGKPIQSKLSIPSDVVLDEGVLY |
| | G₄S + | YSMTINDEQNDIKDEDKGESIITIGEFATVRATRHYVNQDAPF |
| | SNLQGGLRQPR + | GVIHLDITTENGTKTYSYNRKEGEFAINWLVPIGEDSPASIKIS |
| | hGH | VDELDQQRNIIEVPKLYSIDLDNQTLEQWKTQGNVSFSVTRPE |
| | | HNIAISWPSVSYKAAQKEGSRHKRWAHWHTGLALCWLVPM |
| | | DAIYNYITQQNCTLGDNWFGGSYETVAGTPKVITVKQGIEQK |
| | | PVEQRIHFSKGGGGGSNLQGGLRQPRFPTIPLSRLFDNAMLRA |
| | | HRLHQLAFDTYQEFEEAYIPKEQKYSFLQNPQTSLCFSESIPTP |
| | | SNREETQQKSNLELLRISLLLIQSWLEPVQFLRSVFANSLVYG |
| | | ASDSNVYDLLKDLEEGIQTLMGRLEDGSPRTGQIFKQTYSKF |
| | | DTNSHNDDALLKNYGLLYCFRKDMDKVETFLRIVQCRSVEG |
| | | SCGF |
| 35 | hGH | FPTIPLSRLFDNAMLRAHRLHQLAFDTYQEFEEAYIPKEQKYS |
| | | FLQNPQTSLCFSESIPTPSNREETQQKSNLELLRISLLLIQSWLE |
| | | PVQFLRSVFANSLVYGASDSNVYDLLKDLEEGIQTLMGRLED |
| | | GSPRTGQIFKQTYSKFDTNSHNDDALLKNYGLLYCFRKDMD |
| | | KVETFLRIVQCRSVEGSCGF |
| 36 | M+hGH | MFPTIPLSRLFDNAMLRAHRLHQLAFDTYQEFEEAYIPKEQKY |
| | | SFLQNPQTSLCFSESIPTPSNREETQQKSNLELLRISLLLIQSWL |
| | | EPVQFLRSVFANSLVYGASDSNVYDLLKDLEEGIQTLMGRLE |
| | | DGSPRTGQIFKQTYSKFDTNSHNDDALLKNYGLLYCFRKDM |
| | | DKVETFLRIVQCRSVEGSCGF |
| 37 | IL-22 | APISSHCRLDKSNFQQPYITNRTFMLAKEASLADNNTDVRLIG |
| | | EKLFHGVSMSERCYLMKQVLNFTLEEVLFPQSDRFQPYMQE |
| | | VVPFLARLSNRLSTCHIEGDDLHIQRNVQKLKDTVKKLGESG |
| | | EIKAIGELDLLFMSLRNACI |
| 38 | IL-10 | SPGQGTQSENSCTHFPGNLPNMLRDLRDAFSRVKTFFQMKDQ |
| | | LDNLLLKESLLEDFKGYLGCQALSEMIQFYLEEVMPQAENQD |
| | | PDIKAHVNSLGENLKTLRLRLRRCHRFLPCENKSKAVEQVKN |
| | | AFNKLQEKGIYKAMSEFDIFINYIEAYMTMKIRN |
| 39 | N-terminus of | VEDE |
| | a natural | |
| | Cholix | |
| | variant | |
| 40 | N-terminus of | VEEA |
| | a non-natural | |
| | Cholix | |
| | variant | |
| 41 | N-terminus of | LEEA |
| | a non-natural | |
| | Cholix | |
| | variant | |
| 42 | Consensus for | X1-E-X3-X4-L-X6-I-F-D-E-C-R-S-P-C-X16-L-T-P-E-X21-G-K- |
| | cholix | X24-I-Q-S-K-L-X30-I-P-X33-D-V-V-L-D-E-G-V-L-Y-Y-S-M-T-I- |
| | domain I | N-D-E-Q-N-D-I-X56-D-E-X59-K-G-E-S-I-I-T-X67-G-E-F-A-T- |
| | | X73-R-A-T-R-H-Y-V-X81-Q-D-A-P-F-G-V-I-X90-L-D-I-T-T-E-N- |
| | | G-T-K-X101-Y-S-X104-N-R-K-X108-X109-E-F-X112-I-X114-W- |
| | | L-V-X118-X119-G-E-D-S-P-A-S-I-K-I-S-X131-D-E-X134-D-Q- |
| | | X137-R-N-I-I-E-V-P-K-L-Y-S-I-D-L-D-N-Q-T-L-E-Q-W-X160- |
| | | X161-Q-G-N-V-X166-F-X168-V-T-R-P-E-X174-X175-I-A-I-S-W- |
| | | P-S-V-S-Y-X186-A-A-X189-K-X191-G-X193-R-H-K-R-W-A- |
| | | X200-W-X202-T-X204-X205-X206-X207-X208-X209-L-X211- |
| | | X212-X213-X214-X215-X216-X217-X218-X219-X220-X221- |
| | | X222-X223-X224-C-T-X227-G-X229-X230-W-X232-G-G-X235-Y- |
| | | X237-T-V-A-G-X242-P-X244-X245-I-X247-V-K-Q-G-X252-E-Q- |
| | | K-X256-V-E-Q-R-I-H-F-S-X265; wherein X1 is selected from the |
| | | group consisting of V and L; X3 is selected from the group |
| | | consisting of E and D; X4 is selected from the group consisting of A |
| | | and E; X6 is selected from the group consisting of N and K; X16 is |
| | | selected from the group consisting of S and L; X21 is selected from |
| | | the group consisting of P and L; X24 is selected from the group |
| | | consisting of P and Q; X30 is selected from the group consisting of S |
| | | and F; X33 is selected from the group consisting of S and G; X56 is |
| | | selected from the group consisting of K and M; X59 is selected from |
| | | the group consisting of D and G; X67 is selected from the group |
| | | consisting of I and F; X73 is selected from the group consisting of V |
| | | and I; X81 is selected from the group consisting of N and S; X90 is |
| | | selected from the group consisting of H and N; X101 is selected from |
| | | the group consisting of T and M; X104 is selected from the group |
| | | consisting of Y and F; X108 is selected from the group consisting of |
| | | E and D; X109 is selected from the group consisting of G and S; |
| | | X112 is selected from the group consisting of A and T; X114 is |
| | | selected from the group consisting of N and H; X118 is selected from |
| | | the group consisting of P and I; X119 is selected from the group |
| | | consisting of I and P; X131 is selected from the group consisting of |
| | | V and I; X134 is selected from the group consisting L and I; X137 is |
| | | selected from the group consisting Q and K; X160 is selected from |
| | | the group consisting K and E; X161 is selected from the group |
| | | consisting T and N; X166 is selected from the group consisting S and |
| | | F; X168 is selected from the group consisting S and A; X174 is |
| | | selected from the group consisting H and Q; X175 is selected from |
| | | the group consisting N, S, SIAKQS, and SIAKQSIAKQS; X186 is |
| | | selected from the group consisting of K and N; X189 is selected from |
| | | the group consisting of Q, E, and H; X191 is selected from the group |
| | | consisting of E, N, and D; X193 is selected from the group consisting |
| | | of S and A; X200 is selected from the group consisting of H and N; |
| | | X202 is selected from the group consisting of H, L, F, and R; X204 |
| | | is selected from the group consisting of G and T; X205 is selected |
| | | from the group consisting of L and S;X206 is selected from the group |
| | | consisting of A and P; X207 is selected from the group consisting of |
| | | L, E, and K;X208 is selected from the group consisting of C and V; |
| | | X209 is selected from the group consisting of W, V, and T; X211 is |
| | | selected from the group consisting of V and no amino acid; X212 is |
| | | selected from the group consisting of P and no amino acid; X213 is |
| | | selected from the group consisting of M, I, L, and no amino acid; |
| | | X214 is selected from the group consisting of D and no amino acid; |
| | | X215 is selected from the group consisting of A and no amino acid; |
| | | X216 is selected from the group consisting of I and no amino acid; |
| | | X217 is selected from the group consisting of Y and C; X218 is |
| | | selected from the group consisting of N and F; X219 is selected from |
| | | the group consisting of Y and F; X220 is selected from the group |
| | | consisting of I and E; X221 is selected from the group consisting of |
| | | T and D; X222 is selected from the group consisting of Q and P; |
| | | X223 is selected from the group consisting of Q, E, and A; X224 is |
| | | selected from the group consisting of N, L, and Q; X227 is selected |
| | | from the group consisting of L and Y; X229 is selected from the |
| | | group consisting of D and E; X230 is selected from the group |
| | | consisting of N and D; X232 is selected from the group consisting of |
| | | F, H, and Y; X235 is selected from the group consisting of S and A; |
| | | X237 is selected from the group consisting of E and K; X242 is |
| | | selected from the group consisting of T and I; X244 is selected from |
| | | the group consisting of K, E, and G; X245 is selected from the group |
| | | consisting of V and A; X247 is selected from the group consisting of |
| | | T and M; X252 is selected from the group consisting of I and M; |
| | | X256 is selected from the group consisting of P, T, and A; and X265 |
| | | is selected from the group consisting of K, Q, and N. |
| 43 | Vibrio | VEDELNIFDECRSPCSLTPEPGKPIQSKLSIPSDVVLDEGVLYYSMTI |
| | cholerae | NDEQNDIKDEDKGESIITIGEFATVRATRHYVNQDAPFGVIHLDITTE |
| | (Cholix) | NGTKTYSYNRKEGEFAINWLVPIGEDSPASIKISVDELDQQRNIIEVP |
| | DI domain | KLYSIDLDNQTLEQWKTQGNVSFSVTRPEHNIAISWPSVSYKAAQK |
| | | EGSRHKRWAHWHTGLALCWLVPMDAIYNYITQQNCTLGDNWFG |
| | | GSYETVAGTPKVITVKQGIEQKPVEQRIHFSK |
| 44 | Pseudomonas | AEEAFDLWNECAKACVLDLKDGVRSSRMSVDPAIADTNGQGVLH |
| | aeruginosa | YSMVLEGGNDALKLAIDNALSITSDGLTIRLEGGVEPNKPVRYSYT |
| | Exotoxin A | RQARGSWSLNWLVPIGHEKPSNIKVFIHELNAGNQLSHMSPIYTIEM |
| | DI domain | GDELLAKLARDATFFVRAHESNEMQPTLAISHAGVSVVMAQAQPR |
| | | REKRWSEWASGKVLCLLDPLDGVYNYLAQQRCNLDDTWEGKIYR |
| | | VLAGNPAKHDLDIKPTVISHRLHFPE |
| 45 | Aeromonas | ADESFNLWQECATRCTLDLAQGVRASQLDVASLLGGQTDSGVLHY |
| | hydrophila | SMVLEEGGDSLKLALGNALTLRTDGTTITLTSATAGKGPRTYSYTR |
| | Exotoxin A | QGRGNWSLHWLVPVGDDAPASIKVFFHELDAGSEVSHISPIYSIEVS |
| | DI domain | DDLLRTMASNSTLFVRHVENNEINRSLTLSAAGVGFVAAPTQHSRQ |
| | | KRWSEWHTGKVLCLLDPLDAVYNYLSQRTCNLGDTWEGKVYRVL |
| | | AGAPASHDTHIVPTAISHRLHFAK |
| 46 | Chromobacterium | IKTDFEIFKQCADNCILSPAEPGKFISTSLPLQITPSPDEGVLYYSMFV |
| | haemolyticum | QDRFAAAANNSATIKIDEFAKVRINDGQGTGHAPGTLTIELATPDG |
| | mART | KVKKFTHKRRTEWFTLNWVVPIGKDAPTSIKLFIMDMDSNKKIVDH |
| | DI domain | SPLYSVDLDDAALARWPDKAKLAFSSANPRNDIILSWPGVGYTAAP |
| | | TQHNRQKRWSEWHSGILLCWLDPLDAIYNYVTQNRCQLNKTWEG |
| | | KLYQVVAGKPQINEFKPLAKAPIQHRVHFSK |
| 47 | Collimonas | IEGNFRLWDDCSSYCLVYAAPHKIYQTPLATQAVPDSPGKEGVLHY |
| | fungivorans | SMVMKDYVGNGQVLALRLDDFATVFVEQESLRLSLLGSDGKPRNF |
| | mART | QYARQGAKHWSLNWLVPVGDDAPTSIKVFFKNLDGQNNILSISPLY |
| | DI domain | SVEVDDKTLARWPALATFSVTQENVTQGQGLLGIRRAGVSYVAAP |
| | | VNHDRHKRWSEWHSGKLLCLLDPLDAIYNYVSQNRCSLGETWEG |
| | | AIYQTLAGRPVDKYAPPASKPVISQRIHFAK |
| 48 | Shewanella | VNEAFDLWQECATHCQLDLSQGIRSSELDLTPLFETSNEEGILHYSM |
| | putrefacians | LLGEGNEGLKLAIDNALTLHTTHSTINFTSETAESGPRSYSYIRKGEN |
| | mART | NWSLNWLVPVGDDAPASIKIFFLEQDAVGLNRYISPIYSIEVSNNLL |
| | DI domain | NSLAHKSTFYIRAFDNNQTLSMVNISSAGVSYVAAPQQHHRQKRW |
| | | SEWHTGKLLCFLDPFDAFYNYVTQHTCNPDDTWEGQIYRVLAGNP |
| | | ATLDTTAPSTTPAVISHRIHFDR |
| 49 | Janthino- | IKTDFEIFKQCADSCILSPSEPGKFISTSLPVQITPSPDEGVLYYSMFV |
| | bacterium | QDRFASAANNSATIKIDDFAKIRINDGYDTGSAPGSLTIELATPDGQ |
| | lividum | VKKFTHKRRTEWFTLNWAVPIGKDAPTSIKLFIMDIDSNKKIVDHSP |
| | mART | LYSVDLDDAALARWPAKAKLAFSSANAMNDIILSWPGVGYTAAPT |
| | DI domain | QHSRQKRWSEWHSGILLCWLDPLDAIYNYVTQNRCQLKNTWEGSL |
| | | YKVVAGNPQINEFKPVAKAPIQHRVHFSK |
| 50 | Serratia | ANDKFDLWTQCADSCSLDLSNQRIWSSPMDLSLFGQTSEQGVLHFS |
| | fonticola | MVLGENNDSVRIGIDNAFSLLINENSIQFKGVSDITKPVKFNYTRQD |
| | mART | KGNISVNWLVPIGGGTPSEIKVFIHEVNSANQITSMSPIYTIPVSNEVL |
| | DI domain | STLEKKSTIYIRGTNSNSLSYELIVKNAGVSIASTQSGPTRKRRWTH |
| | | WDSGKTLCFIDPLNAIYNYLSQNTCQLDDTWEGKVYLTLYGSPAT |
| | | HDIEMPASPISTRTHFSE |
| 51 | Acinetobacter | AEEAFDLWNECAKACVLDLKDGVRSSRMSVDPAIADTNGQGVLH |
| | baumannii | YSMVLEGGNDALKLAIDNALSITSDGLTIRLEGGVEPNKPVRYSYT |
| | mART | RQARGSWSLNWLVPIGHEKPSNIKVFIHELNAGNQLSHMSPIYTIEM |
| | DI domain | GDELLAKLARDATFFVRAHESNEMQPTLAISHAGVSVVMAQAQPR |
| | | REKRWSEWASGKVLCLLDPLDGVYNYLAQQRCNLDDTWEGKIYR |
| | | VLAGNPAKHDLDIKDNNNSTPTVISHRLHFPE |
| 52 | Pseudomonas | AEEAFDLWNECAKACVLDLKDGVRSSRMSVDPAIADTNGQGVLH |
| | aeruginosa | YSMVLEGGNDALKLAIDNALSITSDGLTIRLEGGVEPNKPVRYSYT |
| | Exotoxin A | RQARGSWSLNWLVPIGHEKPSNIKVFIHELNAGNQLSHMSPIYTIEM |
| | (portion of | GDELLAKLARDATFFVRAHESNEMQPTLAISHAGVSVVMAQAQPR |
| | domain I) | REKR |
| 53 | Aeromonas | ADESFNLWQECATRCTLDLAQGVRASQLDVASLLGGQTDSGVLHY |
| | hydrophila | SMVLEEGGDSLKLALGNALTLRTDGTTITLTSATAGKGPRTYSYTR |
| | Exotoxin A | QGRGNWSLHWLVPVGDDAPASIKVFFHELDAGSEVSHISPIYSIEVS |
| | (portion of | DDLLRTMASNSTLFVRHVENNEINRSLTLSAAGVGFVAAPTQHSRQ |
| | domain I) | KR |
| 54 | Chromobacterium | IKTDFEIFKQCADNCILSPAEPGKFISTSLPLQITPSPDEGVLYYSMFV |
| | haemolyticum | QDRFAAAANNSATIKIDEFAKVRINDGQGTGHAPGTLTIELATPDG |
| | mART | KVKKFTHKRRTEWFTLNWVVPIGKDAPTSIKLFIMDMDSNKKIVDH |
| | (portion of | SPLYSVDLDDAALARWPDKAKLAFSSANPRNDIILSWPGVGYTAAP |
| | domain I) | TQHNRQKR |
| 55 | Collimonas | IEGNFRLWDDCSSYCLVYAAPHKIYQTPLATQAVPDSPGKEGVLHY |
| | fungivorans | SMVMKDYVGNGQVLALRLDDFATVFVEQESLRLSLLGSDGKPRNF |
| | mART | QYARQGAKHWSLNWLVPVGDDAPTSIKVFFKNLDGQNNILSISPLY |
| | (portion of | SVEVDDKTLARWPALATFSVTQENVTQGQGLLGIRRAGVSYVAAP |
| | domain I) | VNHDRHKR |
| 56 | Shewanella | VNEAFDLWQECATHCQLDLSQGIRSSELDLTPLFETSNEEGILHYSM |
| | putrefacians | LLGEGNEGLKLAIDNALTLHTTHSTINFTSETAESGPRSYSYIRKGEN |
| | mART | NWSLNWLVPVGDDAPASIKIFFLEQDAVGLNRYISPIYSIEVSNNLL |
| | (portion of | NSLAHKSTFYIRAFDNNQTLSMVNISSAGVSYVAAPQQHHRQKR |
| | domain I) | |
| 57 | Janthino- | IKTDFEIFKQCADSCILSPSEPGKFISTSLPVQITPSPDEGVLYYSMFV |
| | bacterium | QDRFASAANNSATIKIDDFAKIRINDGYDTGSAPGSLTIELATPDGQ |
| | lividum mART | VKKFTHKRRTEWFTLNWAVPIGKDAPTSIKLFIMDIDSNKKIVDHSP |
| | (portion of | LYSVDLDDAALARWPAKAKLAFSSANAMNDIILSWPGVGYTAAPT |
| | domain I) | QHSRQKR |
| 58 | Serratia | ANDKFDLWTQCADSCSLDLSNQRIWSSPMDLSLFGQTSEQGVLHFS |
| | fonticola | MVLGENNDSVRIGIDNAFSLLINENSIQFKGVSDITKPVKFNYTRQD |
| | mART | KGNISVNWLVPIGGGTPSEIKVFIHEVNSANQITSMSPIYTIPVSNEVL |
| | (portion of | STLEKKSTIYIRGTNSNSLSYELIVKNAGVSIASTQSGPTRKRR |
| | domain I) | |
| 59 | Acinetobacter | AEEAFDLWNECAKACVLDLKDGVRSSRMSVDPAIADTNGQGVLH |
| | baumannii | YSMVLEGGNDALKLAIDNALSITSDGLTIRLEGGVEPNKPVRYSYT |
| | mART | RQARGSWSLNWLVPIGHEKPSNIKVFIHELNAGNQLSHMSPIYTIEM |
| | (portion of | GDELLAKLARDATFFVRAHESNEMQPTLAISHAGVSVVMAQAQPR |
| | domain I) | REKR |
| 60 | Chx197 (VEEA) | VEEALNIFDECRSPCSLTPEPGKPIQSKLSIPSDVVLDEGVLYY |
| | | SMTINDEQNDIKDEDKGESIITIGEFATVRATRHYVNQDAPFG |
| | | VIHLDITTENGTKTYSYNRKEGEFAINWLVPIGEDSPASIKISV |
| | | DELDQQRNIIEVPKLYSIDLDNQTLEQWKTQGNVSFSVTRPEH |
| | | NIAISWPSVSYKAAQKEGSRHKR |
| 61 | Chx197 (LEEA) | LEEALNIFDECRSPCSLTPEPGKPIQSKLSIPSDVVLDEGVLYYS |
| | | MTINDEQNDIKDEDKGESIITIGEFATVRATRHYVNQDAPFGV |
| | | IHLDITTENGTKTYSYNRKEGEFAINWLVPIGEDSPASIKISVD |
| | | ELDQQRNIIEVPKLYSIDLDNQTLEQWKTQGNVSFSVTRPEHN |
| | | IAISWPSVSYKAAQKEGSRHKR |
| 62 | Exenatide | HGEGTFTSDLSKQMEEEAVRLFIEWLKNGGPSSGAPPPS |
| 63 | M_Chx¹⁹⁷- | MVEEALNIFDECRSPCSLTPEPGKPIQSKLSIPSDVVLDEGVLY |
| | Exenatide | YSMTINDEQNDIKDEDKGESIITIGEFATVRATRHYVQQDAPF |
| | | GVIHLDITTEQGTKTYSYNRKEGEFAINWLVPIGEDSPASIKIS |
| | | VDELDQQRNIIEVPKLYSIDLDQQTLEQWKTQGQVSFSVTRPE |
| | | HNIAISWPSVSYKAAQKEGSRHKRHGEGTFTSDLSKQMEEEA |
| | | VRLFIEWLKNGGPSSGAPPPS |
| 64 | M + His₆_PE- | MHHHHHHAEEAFDLWNECAKACVLDLKDGVRSSRMSVDPA |
| | 188-Exenatide | IADTNGQGVLHYSMVLEGGNDALKLAIDNALSITSDGLTIRLE |
| | | GGVEPNKPVRYSYTRQARGSWSLNWLVPIGHEKPSNIKVFIH |
| | | ELNAGNQLSHMSPIYTIEMGDELLAKLARDATFFVRAHESNE |
| | | MQPTLAISHAGVSVVMAQAQPRREKRHGEGTFTSDLSKQME |
| | | EEAVRLFIEWLKNGGPSSGAPPPS |
| 65 | M + His₆_Chx¹⁹⁷- | MHHHHHHVEDELNIFDECRSPCSLTPEPGKPIQSKLSIPSDVVL |
| | WSAAG-N3- | DEGVLYYSMTINDEQNDIKDEDKGESIITIGEFATVRATRHYV |
| | hGH | NQDAPFGVIHLDITTENGTKTYSYNRKEGEFAINWLVPIGEDS |
| | | PASIKISVDELDQQRNIIEVPKLYSIDLDNQTLEQWKTQGNVSF |
| | | SVTRPEHNIAISWPSVSYKAAQKEGSRHKRWSAAGGGGSGG |
| | | GSGGGSFPTIPLSRLFDNAMLRAHRLHQLAFDTYQEFEEAYIP |
| | | KEQKYSFLQNPQTSLCFSESIPTPSNREETQQKSNLELLRISLLL |
| | | IQSWLEPVQFLRSVFANSLVYGASDSNVYDLLKDLEEGIQTL |
| | | MGRLEDGSPRTGQIFKQTYSKFDTNSHNDDALLKNYGLLYCF |
| | | RKDMDKVETFLRIVQCRSVEGSCGF |
| 66 | PE-188- | AEEAFDLWNECAKACVLDLKDGVRSSRMSVDPAIADINGQG |
| | Exenatide | VLHYSMVLEGGNDALKLAIDNALSITSDGLTIRLEGGVEPNKP |
| | | VRYSYTRQARGSWSLNWLVPIGHEKPSNIKVFIHELNAGNQL |
| | | SHMSPIYTIEMGDELLAKLARDATFFVRAHESNEMQPTLAISH |
| | | AGVSVVMAQAQPRREKRHGEGTFTSDLSKQMEEEAVRLFIE |
| | | WLKNGGPSSGAPPPS |
| 67 | Chx¹⁹⁷- | VEDELNIFDECRSPCSLTPEPGKPIQSKLSIPSDVVLDEGVLYY |
| | WSAAG-N3- | SMTINDEQNDIKDEDKGESIITIGEFATVRATRHYVNQDAPFG |
| | hGH | VIHLDITTENGTKTYSYNRKEGEFAINWLVPIGEDSPASIKISV |
| | | DELDQQRNIIEVPKLYSIDLDNQTLEQWKTQGNVSFSVTRPEH |
| | | NIAISWPSVSYKAAQKEGSRHKRWSAAGGGGSGGGSGGGSF |
| | | PTIPLSRLFDNAMLRAHRLHQLAFDTYQEFEEAYIPKEQKYSF |
| | | LQNPQTSLCFSESIPTPSNREETQQKSNLELLRISLLLIQSWLEP |
| | | VQFLRSVFANSLVYGASDSNVYDLLKDLEEGIQTLMGRLEDG |
| | | SPRTGQIFKQTYSKFDTNSHNDDALLKNYGLLYCFRKDMDK |
| | | VETFLRIVQCRSVEGSCGF |
| 68 | Chx¹⁹⁷- | VEEALNIFDECRSPCSLTPEPGKPIQSKLSIPSDVVLDEGVLYY |
| | Exenatide | SMTINDEQNDIKDEDKGESIITIGEFATVRATRHYVQQDAPFG |
| | | VIHLDITTEQGTKTYSYNRKEGEFAINWLVPIGEDSPASIKISV |
| | | DELDQQRNIIEVPKLYSIDLDQQTLEQWKTQGQVSFSVTRPEH |
| | | NIAISWPSVSYKAAQKEGSRHKRHGEGTFTSDLSKQMEEEAV |
| | | RLFIEWLKNGGPSSGAPPPS |

### EXAMPLES

### Example 1—Expression and Purification of Furin-Cleavable Constructs

All proteins were prepared by standard protocols for E. coli expression following codon-optimization. Synthesized genes were cloned into the vector pET-26b (+) DNA expression vector and transformed into E. coli T7 Express cells that were cultured by shake-flask. Cultures were induced with 1 mM IPTG and the temperature reduced to 26° C. for a further 12 h after induction before cells were harvested by centrifugation. Cell pellets were suspended in double distilled water, lysed using two passes of high-pressure using a microfluidizer, and clarified by centrifugation.

### Example 2—In Vitro Cleavage Assay

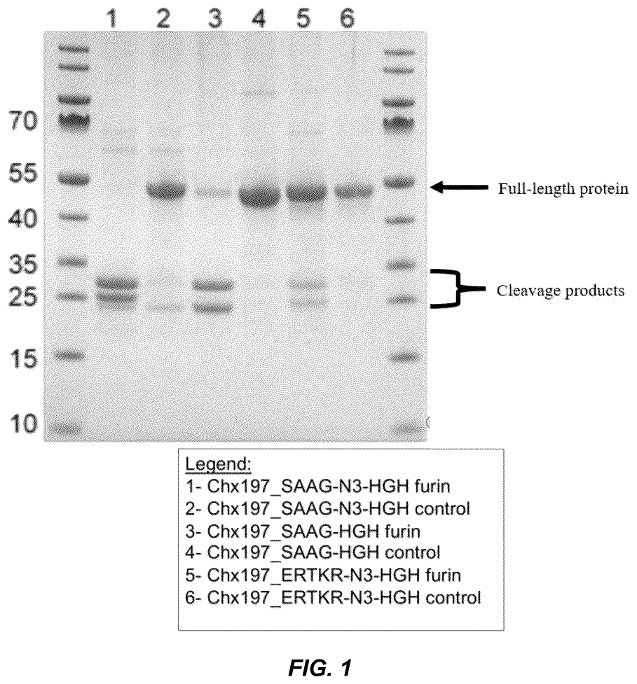

An in vitro cleavage assay was prepared for various delivery constructs (i.e., Chx¹⁹⁷_SAAG-N3-HGH (SEQ ID NO: 12), Chx¹⁹⁷_SAAG-HGH (SEQ ID NO: 11), Chx¹⁹⁷_ERTKR-N3-HGH (SEQ ID NO: 13), where N3 is G₃SG₃SG₃S). Specifically, 25 μg of delivery construct (3 μL) was combined with 21.5 μL solution of 20 mM HEPES pH 5.0, 0.2 mM CaCl₂), and 0.2 mM TCEP. Then 2 μL of furin (2 μg) was added. For controls, 2 μL of the HEPES-containing buffer was added instead of furin. The resulting reaction mixture was allowed to proceed at room temperature overnight (˜18 h). Equal amounts of each reaction mixture (each having a different delivery construct protein) were run on an SDS-PAGE gel under reducing conditions. An image of the resulting gel is shown in FIG. 1, with molecular weight markers shown in lanes 0 and 7. As can be seen from FIG. 1, only furin-containing reaction mixtures resulted in meaningful cleavage of a delivery construct. Further, the Chx¹⁹⁷_SAAG-N3-HGH construct (SEQ ID NO: 12) appeared to have a higher cleavage efficiency than Chx¹⁹⁷_SAAG-HGH (SEQ ID NO: 11), which had a higher cleavage efficiency than Chx¹⁹⁷_ERTKR-N3-HGH (SEQ ID NO: 13).

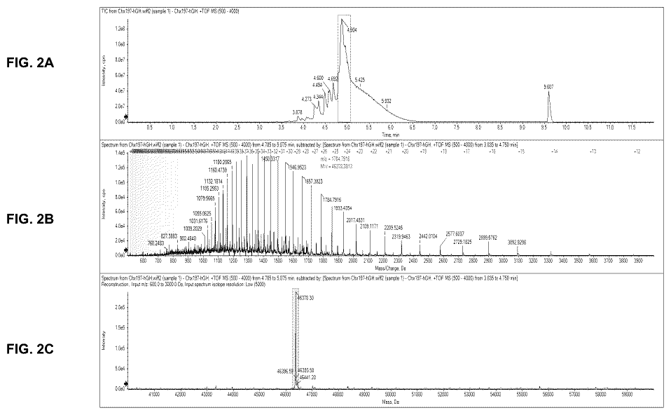

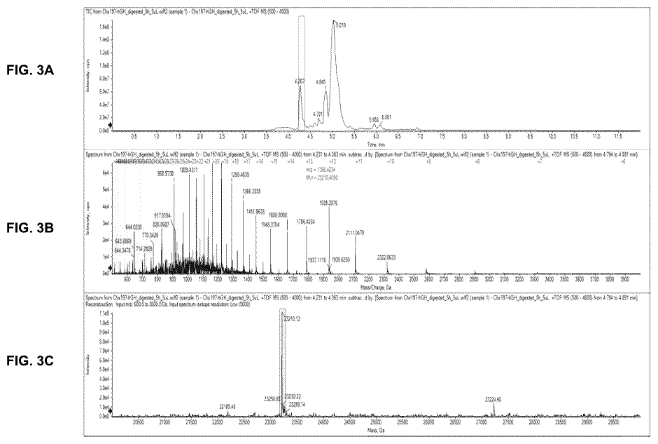

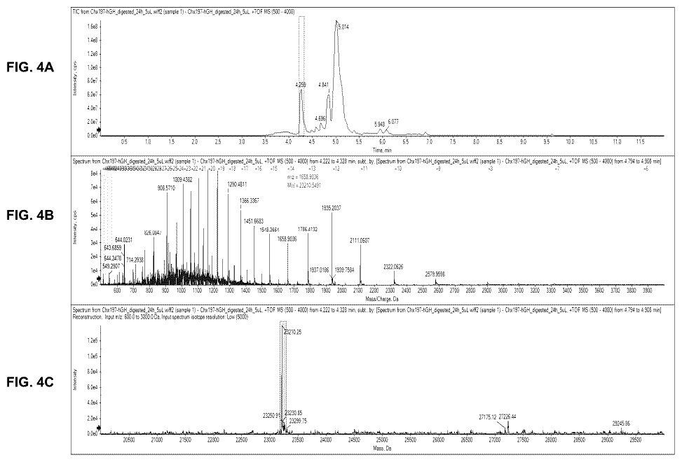

The cleavage site of the Chx¹⁹⁷_SAAG-N3-HGH construct (SEQ ID NO: 12) was analyzed by mass spectrometry and ion chromatogram (SCIEX ZenoTOP 7600). Specifically, 10 μg of the Chx¹⁹⁷_SAAG-N3-HGH construct, 5 μl 10× buffer, 1 μl furin, and 34.6 μl DI H₂O were added together, and the solutions were incubated at 30° C. for 0, 5 or 24 hours. Each resulting reaction mixture was analyzed by mass spectrometry. FIG. 2 shows that at 0 hrs, the Chx¹⁹⁷_SAAG-N3-HGH construct was intact, resulting in a single peak at 46,378.30 Da, the expected weight of the Chx¹⁹⁷_SAAG-N3-HGH. After 5 hrs and 24 hrs, the Chx¹⁹⁷_SAAG-N3-HGH construct was cleaved by furin, resulting in a peak at around 23210.2 Da, the expected weight of amino acids 1-204 of the Chx¹⁹⁷_SAAG-N3-HGH (FIG. 3 and FIG. 4) with the corresponding M+His6 tag. This suggests that the scissile bond in the Chx¹⁹⁷_SAAG-N3-HGH construct is immediately C-terminal of the arginine residue immediately preceding SAAG-N3-HGH.

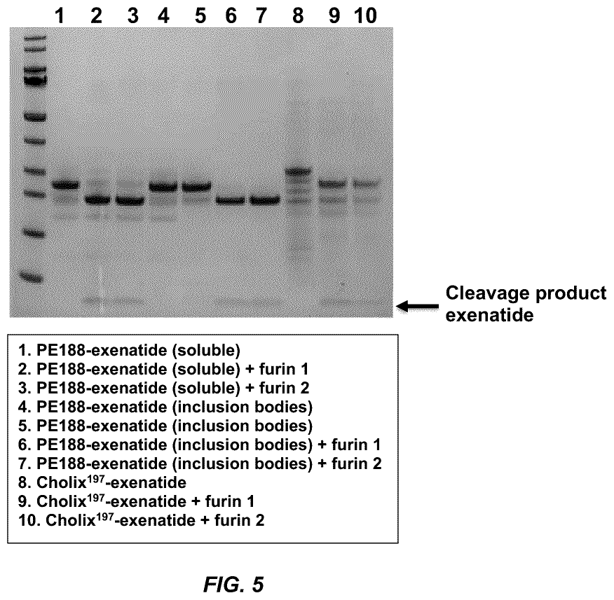

The same in vitro cleavage assay was performed for delivery constructs Chx¹⁹⁷-Exenatide (SEQ ID NO: 63) and PE188-Exenatide (SEQ ID NO:64). Specifically, 25 μg of delivery construct (3 μL) was combined with 21.5 μL solution of 20 mM HEPES pH 5.0, 0.2 mM CaCl₂), and 0.2 mM TCEP. Then 2 μL of furin (2 μg) was added. For controls, 2 μL of the HEPES-containing buffer was added instead of furin. The resulting reaction mixture was allowed to proceed at room temperature overnight (˜18 h). Equal amounts of each reaction mixture (each having a different delivery construct protein) were run on an SDS-PAGE gel under reducing conditions. An image of the resulting gel is shown in FIG. 5, with molecular weight markers shown in lane 0. As shown in FIG. 5, exenatide was cleaved off from a delivery construct only in the furin-containing reaction mixtures. Both Chx¹⁹⁷-Exenatide (SEQ ID NO: 63) and PE188-Exenatide (SEQ ID NO: 64) showed good cleavage efficiency. Moreover, exenatide was released in the PE188-Exenatide soluble fraction and the PE188-Exenatide obtained from inclusion bodies.

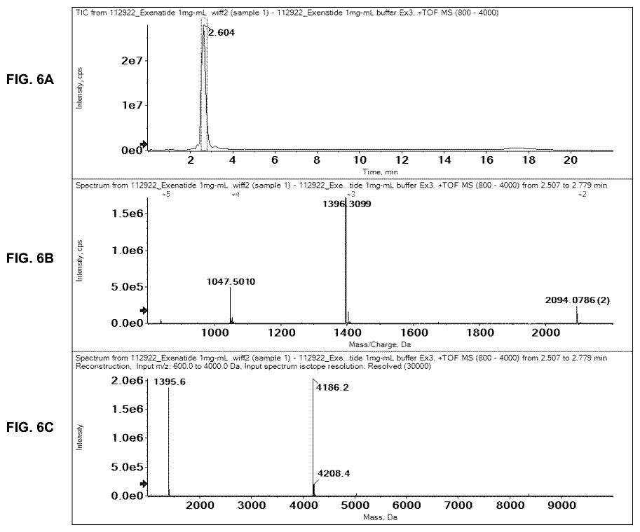

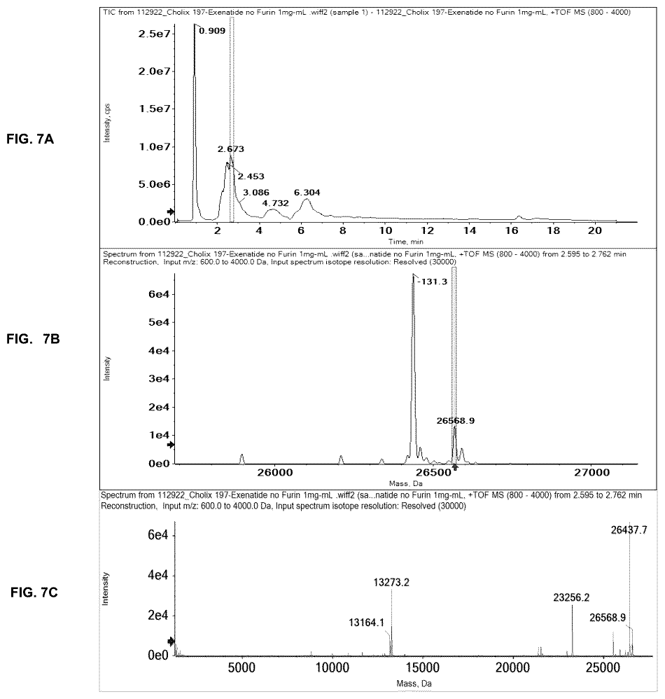

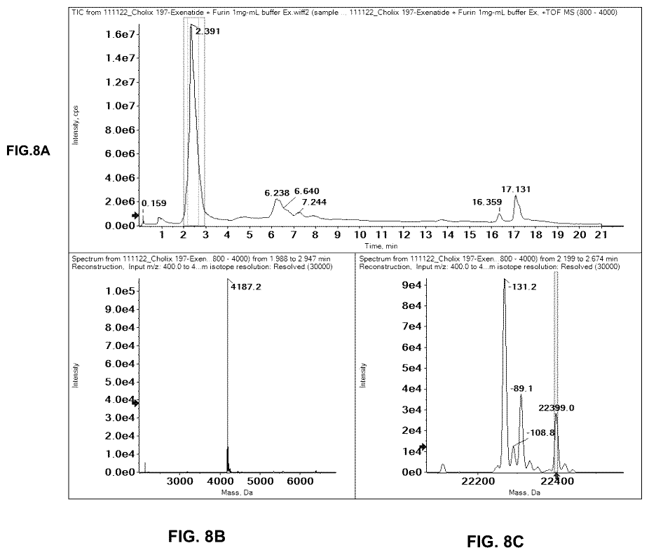

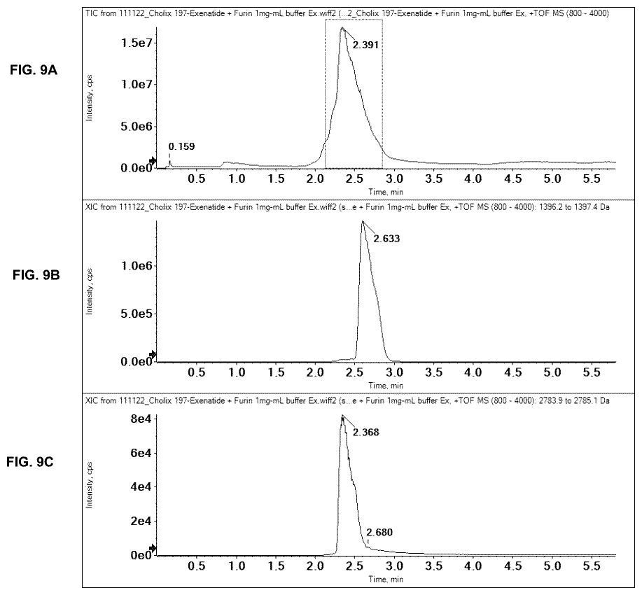

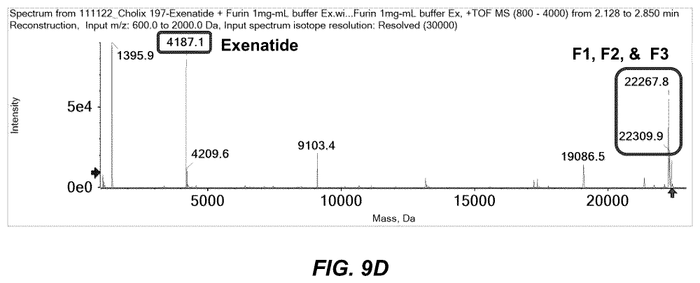

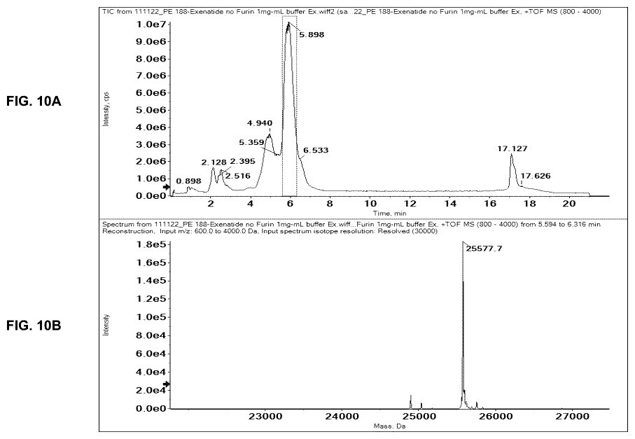

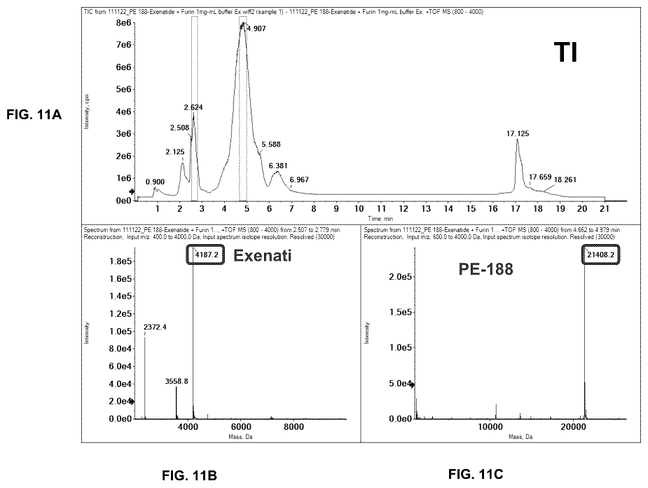

The cleavage sites of exenatide (SEQ ID NO: 62), Chx¹⁹⁷-Exenatide (SEQ ID NO: 63) and the PE188-Exenatide (SEQ ID NO:64) were analyzed by mass spectrometry and ion chromatogram. Specifically, 10 μg of the proteins, 5 μl 10× buffer, 1 μl furin, and 34.6 μl DI H₂O were added together, and the solutions were incubated at 30° C. for 0, 5 or 24 hours. For controls, 1 μL of DI H₂O was added instead of furin, and the solutions were incubated at 30° C. FIG. 6 shows that exenatide incubated with furin remained intact, resulting in a principal peak at expected intensity for exenatide in both ion chromatogram (FIG. 6A) and mass spectrometry (FIGS. 6B and 6C). FIG. 7 shows that Cholix-197-Exenatide without furin has no peak at the expected weight of exenatide, suggesting that exenatide was not released from Cholix-197-Exenatide delivery construct. On the contrary, Exenatide was released from Cholix-197-Exenatide after incubating with furin for 2 hours (FIG. 8). The ion chromatogram (FIG. 9A) shows that the peak around 2.391 cps contains two main peaks, one peak corresponding to the Cholix-197 fragment (FIG. 9B) and one peak corresponding to the exenatide fragment (FIG. 9C). The mass spectrometry (FIG. 9D) also shows one peak at the expected weight of exenatide (4187.2 Da) and one peak at the expected weight of amino acids 1-198 of the Cholix-197-exenatide construct (22399.0 Da). The mass spectrometry demonstrates that the scissile bond in the Chx¹⁹⁷-exenatide construct is immediately C-terminal of the arginine residue immediately preceding exenatide.

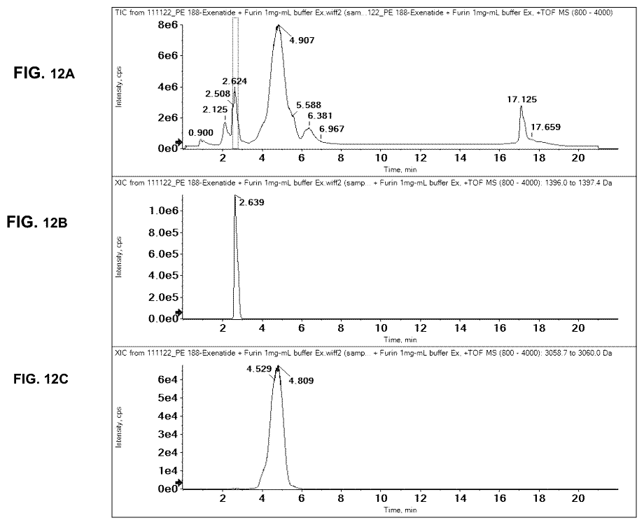

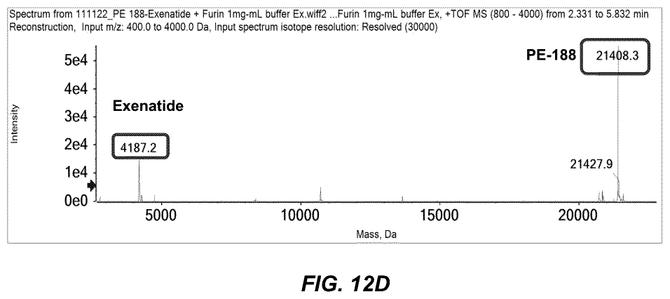

Similarly, PE-188-Exenatide without furin remained intact (FIG. 10), while exenatide was released from PE-188-Exenatide after incubating the delivery construct with furin for 2 hours (FIG. 11). The ion chromatogram (FIG. 12) shows one peak corresponding to the Cholix-197 fragment (FIG. 12C) and one peak corresponding to the exenatide fragment (FIG. 12B). The mass spectrometry (FIG. 12D) also shows one peak at the expected weight of exenatide (4187.2 Da) and one peak at the expected weight of amino acids 1-193 of the PE188-exenatide construct (22399.0 Da). The mass spectrometry demonstrates that the scissile bond in the PE188-exenatide construct is immediately C-terminal of the arginine residue immediately preceding exenatide.

### Example 3—In Vitro Transport Assays

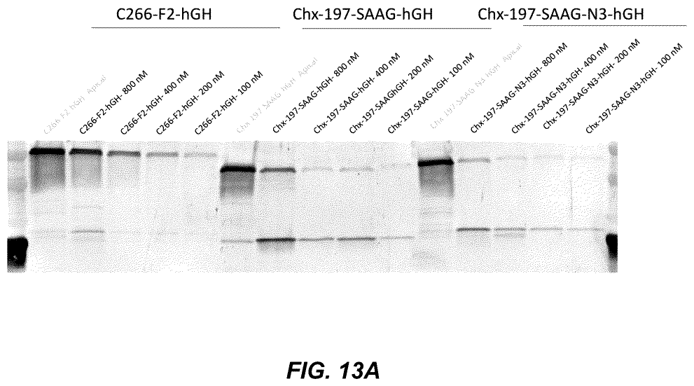

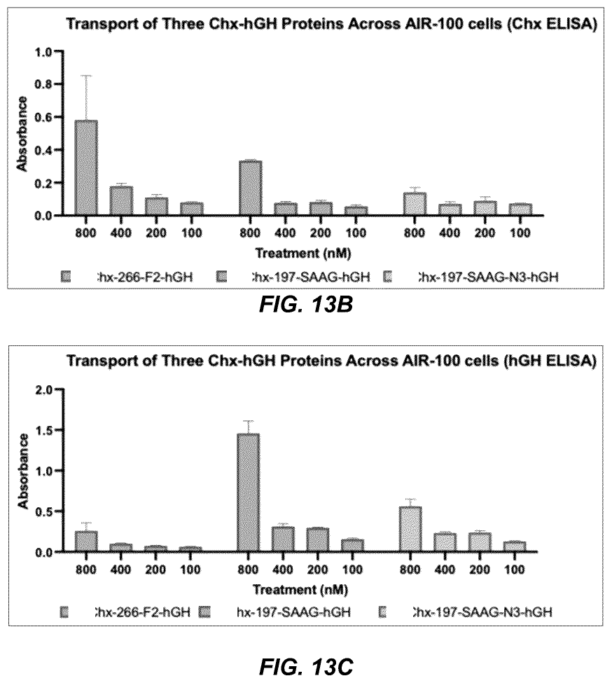

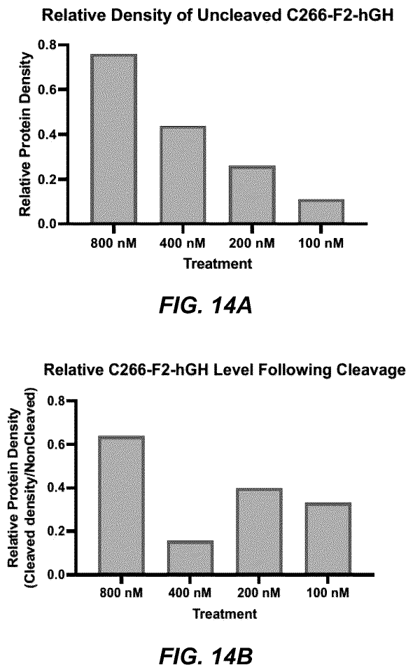

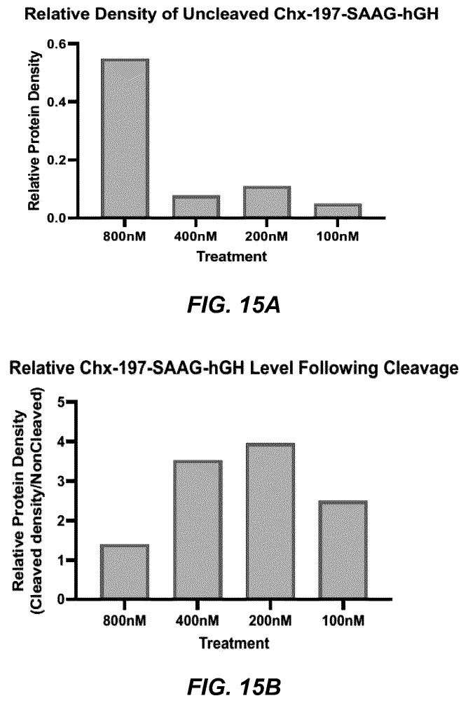

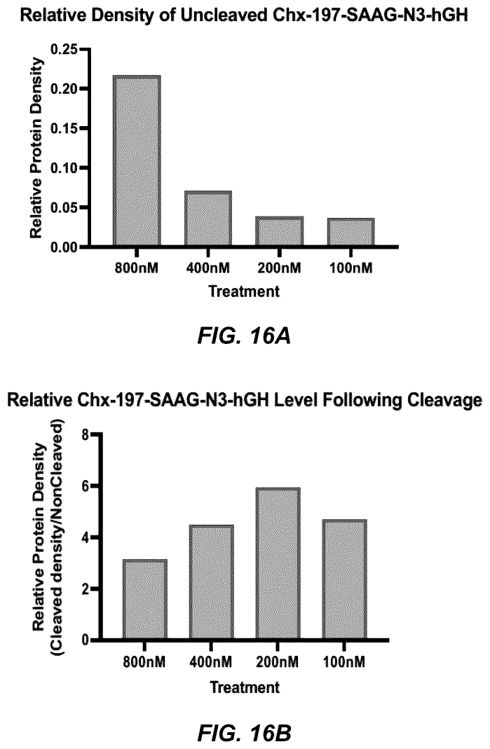

Human 3D in vitro small intestinal tissue model (EpiIntestinal SMI-100) and EpiAirway AIR-100 monolayers were purchased from MatTek Corporation (Ashland, MA). Following 24 hours incubation at 37° C. and 5% CO₂, transepithelial electric resistance (TEER) was measured for monolayer integrity. Inserts with a TEER>400 were used. Dextran (70 kD) was also used as a marker for negative transport and insert integrity. The chambers were washed once with transport buffer (PBS). 100 μl of PBS containing a cholix-containing fusion protein or a control protein (e.g., human growth hormone) at the same molarity (400 nM) was added to the apical side, and 500 μl of PBS was added to basolateral chambers. The amount of protein in the basal solution was analyzed by Western blotting (FIG. 13A) and ELISA (FIGS. 13B and 13C) for its transport efficiency and amount. An anti-hGH polyclonal antibody (AF1067; R&D Systems) was used for the Western blot. As shown in FIGS. 13A-C, a dose-dependent cleavage response was observed at 2 h in all three constructs when using EpiAirway AIR-100 cells, with Chx¹⁹⁷-SAAG-hGH (SEQ ID NO: 11) and Chx¹⁹⁷-SAAG-N3-hGH (SEQ ID NO: 12) showing a higher cleavage response than Chx²⁶⁶-F2-hGH (SEQ ID NO: 34), suggesting that the furin sites on Chx¹⁹⁷-SAAG-hGH (SEQ ID NO: 11) and Chx¹⁹⁷-SAAG-N3-hGH (SEQ ID NO: 12) are more accessible for protease cleavage than the corresponding site for Chx²⁶⁶-F2-hGH (SEQ ID NO: 34). Bar graphs quantifying the intensity of bands from the images of the experiment shown in FIG. 13A are set forth in FIGS. 14A, 14B, 15A, 15B, 16A, and 16B.

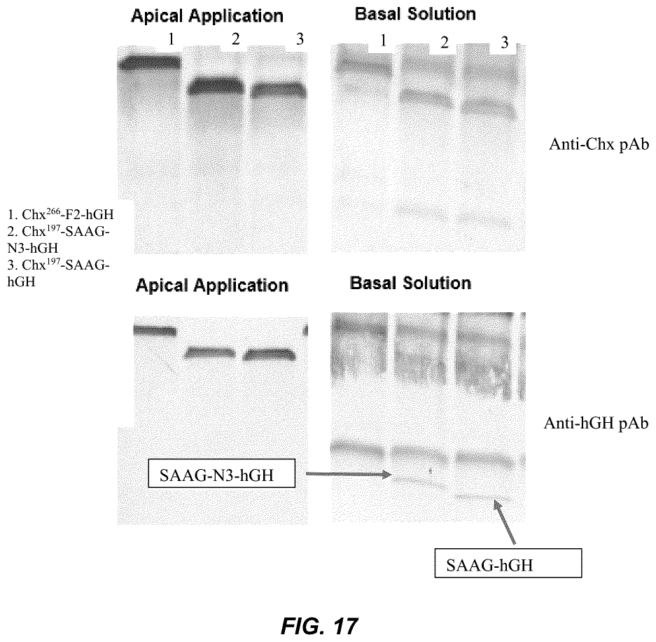

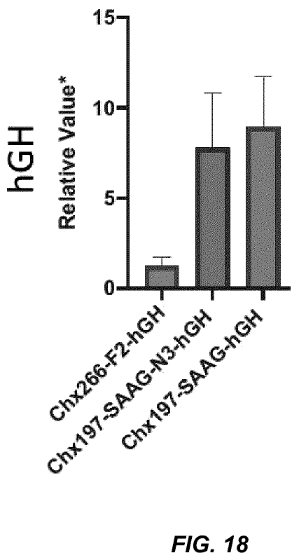

Similar results were obtained from an analogous experiment (2 h) with SMI-100 cells (FIG. 17 and FIG. 18). Specifically, FIG. 17 shows the cleavage of various constructs that are initially delivered to the apical side of SMI-100 cells as they transcytose to the basal side, while FIG. 18 shows the relative value of human growth hormone as determined from an ELISA assay in SMI-100 cells in which cholix-containing fusion protein was initially added to the apical side of the SMI-100 cells and PBS was added to the basolateral side.

### Example 4—In Vitro Enzymatic Digestion of Fusion Proteins by Furin Produces the Same Fragmentation and Cleavage Sites as the Transported Fusion Proteins Across SMI-100 Cells

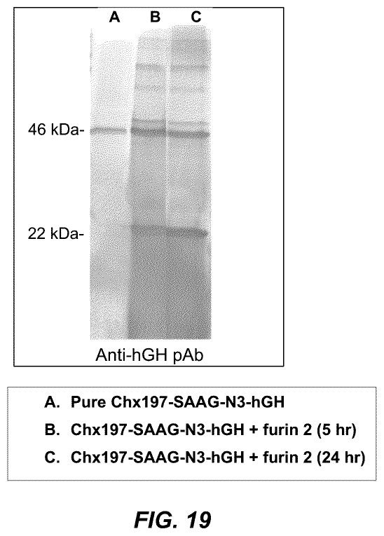

The in vitro cleavage assay and the in vitro transport assay were prepared for delivery construct Chx¹⁹⁷-SAAG-N3-hGH (SEQ ID NO: 12). Specifically, the in vitro cleavage assay was performed by combining 25 μg of delivery construct (3 μL) with 21.5 μL solution of 20 mM HEPES pH 5.0, 0.2 mM CaCl₂), and 0.2 mM TCEP. Then 2 μL of furin (2 μg,) was added. For controls, 2 μL of the HEPES-containing buffer was added instead of furin. The resulting reaction mixture was allowed to proceed at room temperature overnight (˜18 h). The amount of protein in the solution was analyzed by Western blotting. An anti-hGH polyclonal antibody (AF1067; R&D Systems) was used for the Western blot. As shown in FIG. 19, Chx¹⁹⁷-SAAG-N3-hGH was cleaved to release the hGH. Similar amounts of cleavage were observed after incubating with furin for 5 hours and for 24 hours.

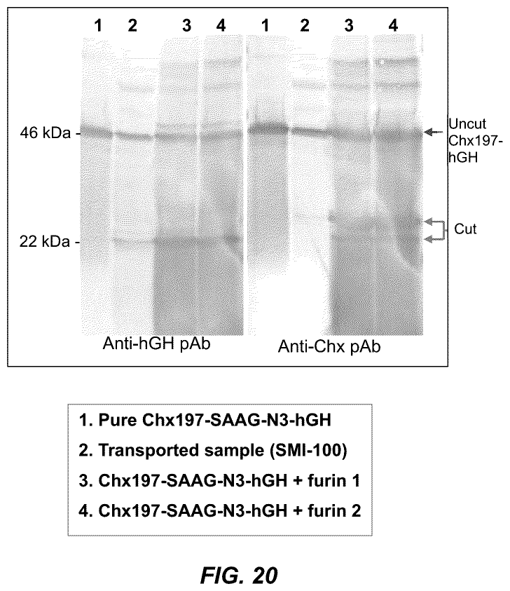

Human 3D in vitro small intestinal tissue model (EpiIntestinal SMI-100) was used for the in vitro transport assay. Following 24 hours incubation at 37° C. and 5% CO₂, transepithelial electric resistance (TEER) was measured for monolayer integrity. Inserts with a TEER>400 were used. Dextran (70 kD) was also used as a marker for negative transport and insert integrity. The chambers were washed once with transport buffer (PBS). 100 μl of PBS containing 400 nM Chx¹⁹⁷-SAAG-N3-hGH was added to the apical side, and 500 μl of PBS was added to basolateral chambers. Two commercially available furins, furin 1 (1503SE; R&D Systems) and furin 2 (P8077S; New England BioLabs), were used to test the cleave efficiency. The protein in the basal solution together with the protein products after in vitro enzymatic digestion of Chx¹⁹⁷. SAAG-N3-hGH by furin were analyzed by Western blotting (FIG. 20). An anti-hGH polyclonal antibody (AF1067; R&D Systems) and an anti-Chx²⁶⁶ polyclonal antibody (Antibody Solutions) were used for the Western blot. As shown in FIG. 20, bands at 22 kDa correlating to the weight of hGH were observed in the basal solution after Chx¹⁹⁷-SAAG-N3-hGH was transported across SMI-100 cells and in the protein products after in vitro enzymatic digestion of Chx¹⁹⁷-SAAG-N3-hGH by furin. This suggests that the in vitro transported Chx¹⁹⁷-SAAG-N3-hGH across SMI-100 cells released the same fragment of hGH as the in vitro enzymatic digestion of Chx¹⁹⁷-SAAG-N3-hGH.

### Example 5—In Vivo Transport Assays

### Immunofluorescence Microscopy and Serum Analysis Following Wistar Rat Intraluminal Injection

Male Wistar rats, housed 3-5 per cage with a 12/12 h light/dark cycle, were 225-275 g (approximately 6-8 weeks old) at the beginning of this study. All experiments were conducted during the light phase using a non-recovery protocol that used continuous isoflurane anesthesia. A 4-5 cm midline abdominal incision exposed mid-jejunum regions. Stock solutions at 3.86×10⁻⁵ M of test articles were prepared in PBS, with 50 μL (per 250 g rat) being administered by intraluminal injection (ILI) using a 29-gauge needle. The injection site mesentery was marked with a permanent marker. Fifteen minutes after administration, a 3-5 mm region that captured the marked intestine segment was isolated and processed for microscopic assessment.

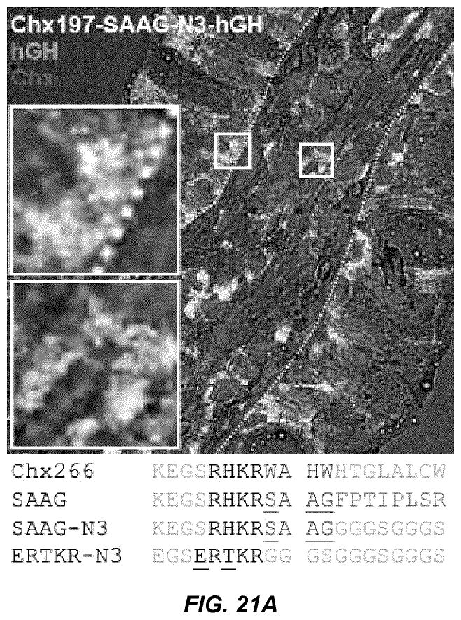

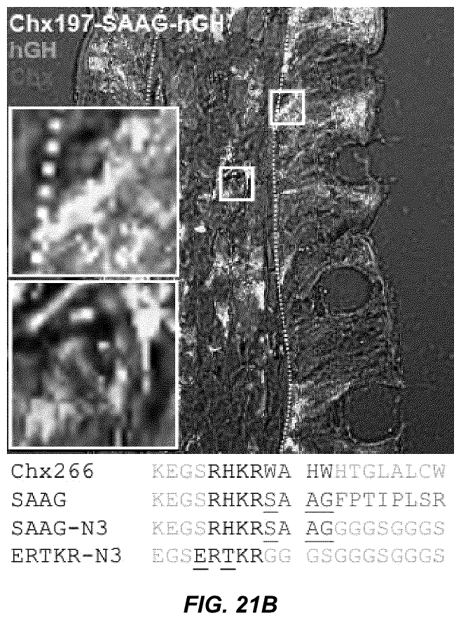

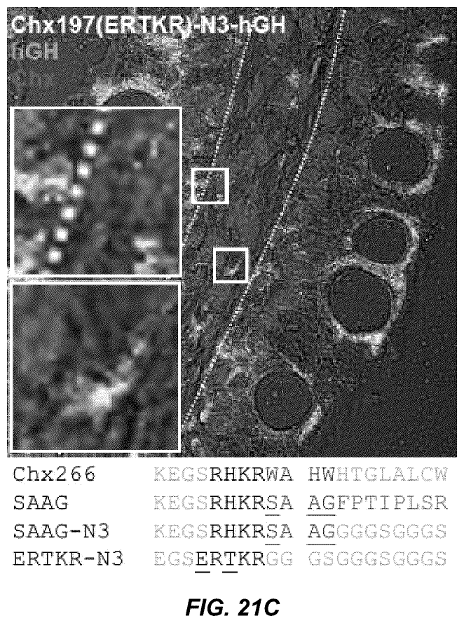

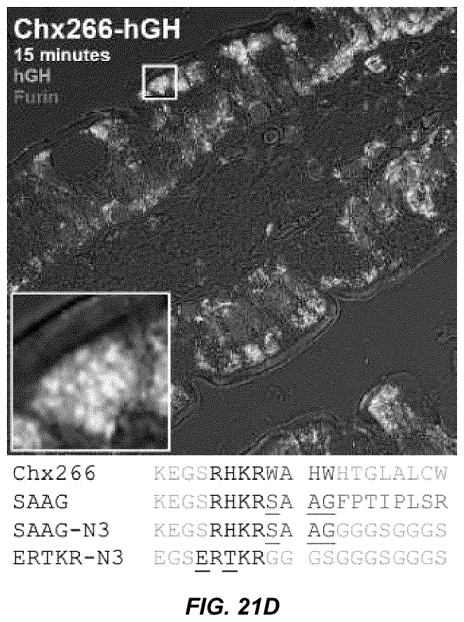

Specifically, isolated intestinal tissues were captured 15 minutes after administration, and the tissues were fixed in 4% paraformaldehyde at 4° C. for 18-24 h, processed using a Leica TP1020 tissue processor, dehydrated in increasing concentrations of ethanol, cleared with HistoClear (National Diagnostics), and infused with molten paraffin wax. Sections cut from tissue-embedded paraffin wax blocks (5 μm thickness; Jung Biocut2035 microtome) were mounted on glass microscope slides, rehydrated, and processed for antigen retrieval by boiling slides in 10 mM sodium citrate for 10 min followed by washing with PBS. Processed tissue slices were permeabilized using 0.1% Triton X-100 in PBS for 30 min, blocked using 2% donkey or goat serum and 2% BSA in 0.1% Triton X-100 in PBS for 2 h, and incubated overnight at 4° C. with primary antibodies diluted in 1% BSA and 0.05% Triton X-100 in PBS at 4° C. Tissue slices were washed thrice with PBS, incubated for 2 h with secondary antibodies conjugated to AlexaFluor® fluorescent dyes, washed thrice with PBS, and incubated for 1 h in 200 nM DAPI, washed with PBS, dehydrated in ethanol, and covered by mounting a coverslip with Fluorshield (Abcam) mounting media; all steps performed at room temperature unless otherwise noted. After allowing the mounting media to dry at 4° C. overnight, fluorescent images were obtained using a Zeiss 880 LSM confocal microscope, a Perkin Elmer Vectra, or a Leica SP5 inverted confocal microscope using the following settings. Excitation/emission wavelengths used for the various fluorophores were: DAPI (405 nm/462 nm), Alexafluor 488 (488 nm/562 nm), Alexafluor 564 (561 nm/602 nm), and Alexafluor 633 (633 nm/693 nm). The pinhole was set between 0.99-1.22 airy units to achieve a z-resolution of 0.8 μm. All laser intensities were set at 2%. All images were captured with a total magnification of 100λ-630λ. Image analytics software included Zeiss Zen 2.6 Blue software, Leica SP5, Perkin Elmer Phenochart and Inform, Imaris, and ImageJ. The resulting images for four delivery constructs (Chx¹⁹⁷-SAAG-N3-hGH (SEQ ID NO: 12), Chx¹⁹⁷-SAAG-hGH (SEQ ID NO: 11), Chx¹⁹⁷(ERTKR)-N3-hGH (SEQ ID NO: 13), and Chx²⁶⁶-F2-hGH (SEQ ID NO: 34)) are shown in FIGS. 21A-21D. All four delivery constructs were taken up into the cell within 15 minutes. SAAG-containing constructs (SEQ ID NO: 11 and SEQ ID NO: 12) showed improved cargo release (e.g., relative to the Chx¹⁹⁷(ERTKR)-N3-hGH (SEQ ID NO: 13)) as detected in systemic circulation. The constructs having a carrier that terminated at position 197 were cleaved more efficiently that the construct having a carrier that terminated at position 266. Substantial cleavage did not occur within the epithelial cells, but separation of hGH from the cholix carrier is observed in the lamina propria. Transport of the ERTKR construct (SEQ ID NO: 13) was significantly reduced relative to Chx¹⁹⁷-SAAG-N3-hGH (SEQ ID NO: 12) and Chx¹⁹⁷-SAAG-hGH (SEQ ID NO: 11), and little separation of hGH from the cholix carrier was observed.

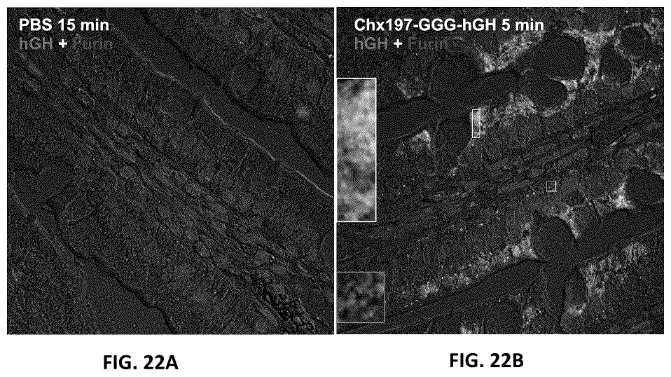

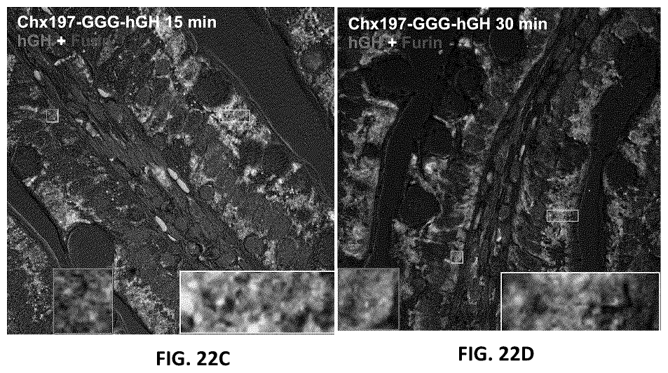

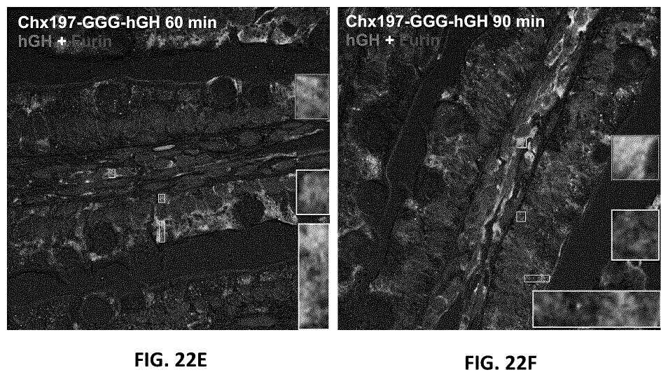

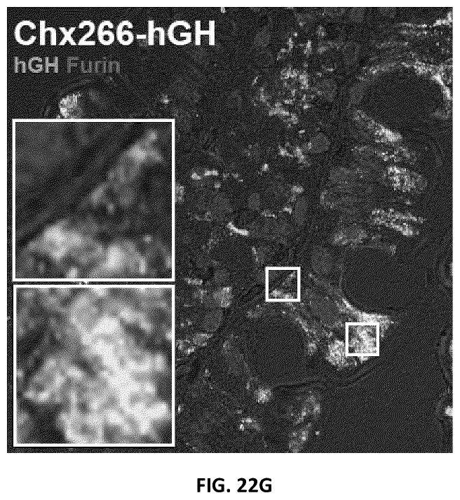

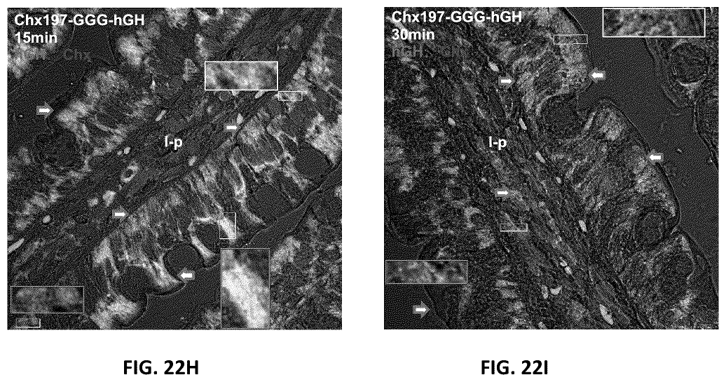

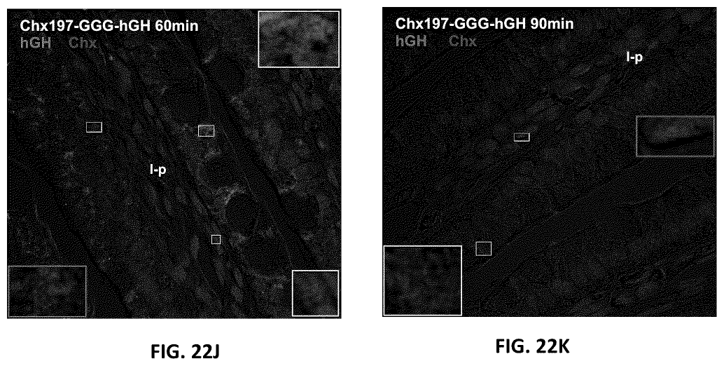

The resulting images for the delivery construct Chx¹⁹⁷-GGG-hGH (SEQ ID NO: 10) are shown in FIG. 22. Within 5 minutes, cleavage of hGH from Chx¹⁹⁷-GGG-hGH started, and some of the released hGH reached basal side of epithelial cells (FIG. 22B). The level of hGH continued to increase, and hGH mainly observed in the basal parts of epithelial cells 15 minutes after injecting Chx¹⁹⁷-GGG-hGH (FIG. 22C). Co-localization of Cholix and hGH was observed mainly in the apical part (FIG. 22H). In some basal parts, only hGH was seen, suggesting that cleavage had already started to occur at this point (FIG. 22H). The amount of released hGh was increased relative to the hGH in the delivery construct. At 30 minutes and 60 minutes after injecting the delivery construct, it was observed that furin interacted with Chx¹⁹⁷-GGG-hGH in the apical part of the epithelial cells and the interaction continued in the basal part (FIG. 22D and FIG. 22E). Partial colocalization of furin and Chx¹⁹⁷-GGG-hGH was also observed in epithelial cells and in lamina propria. At 30 mins post-injection, most hGH had already been released from Chx¹⁹⁷-GGG-hGH (FIG. 22I). Cholix reached lamina propria, while most of the hGH was in epithelial cells (FIG. 22I). At 60 minutes post-injection, lower hGH levels were observed as hGH was redistributed into the systemic circulation, and Cholix was no longer present in lamina propria (FIG. 22J). At 90 minutes after the injection, colocalization of furin and hGH was complete and hGH level was diminished (FIG. 22F and FIG. 22K). Similar interaction of furin with Chx²⁶⁶-F2-hGH (SEQ ID NO: 34) in the apical compartment of the epithelial cells was observed (FIG. 22G).

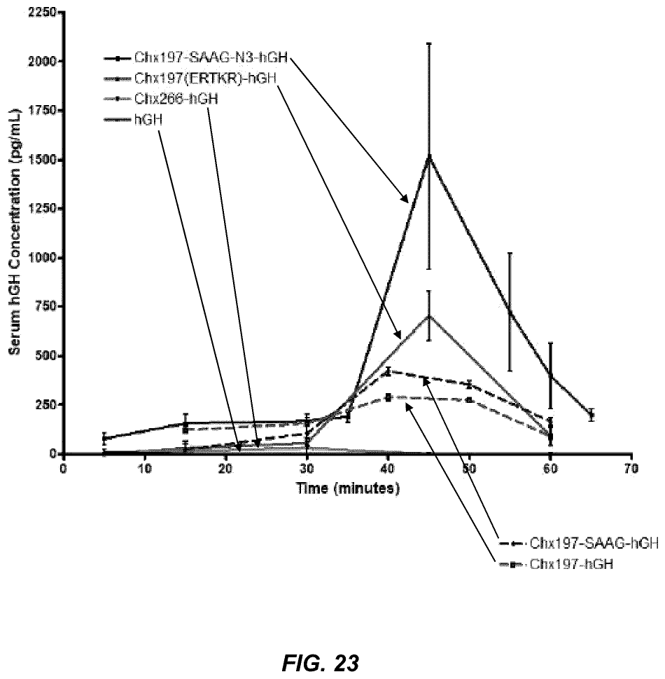

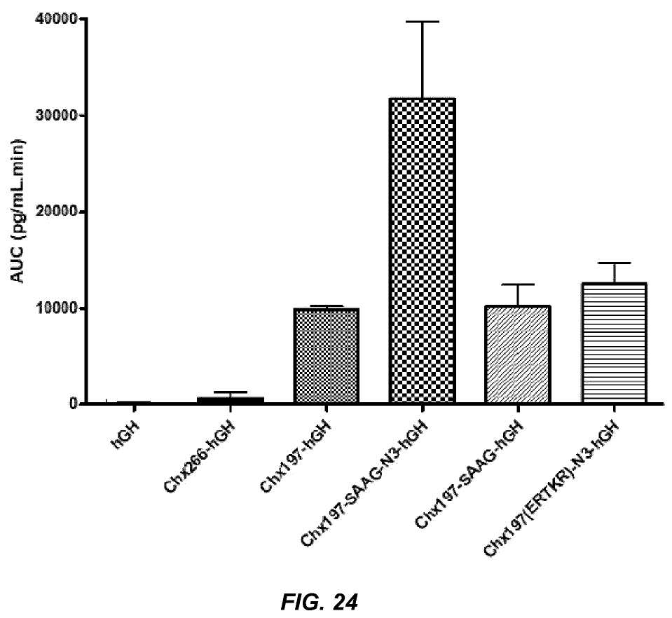

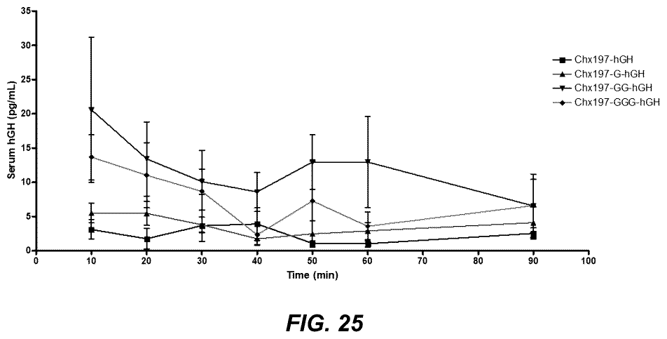

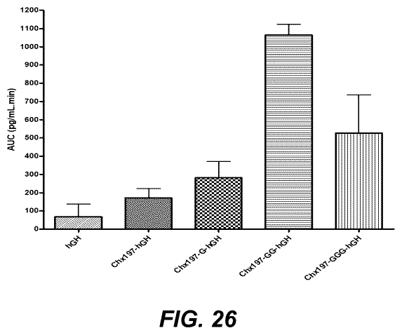

Also, serum samples from the Wistar rats involved in this study were obtained at different time points after administration and analyzed for hGH concentration and cholix concentration. The results are shown in FIGS. 23-26. Specifically, FIGS. 23 and 25 provide serum concentrations of hGH (mean±standard deviation) over time as measured by ELISA. FIGS. 24 and 26 provide the AUC values for each construct. Chx¹⁹⁷-hGH (SEQ ID NO: 7), Chx¹⁹⁷-SAAG-hGH (SEQ ID NO: 11), and Chx¹⁹⁷(ETKTR)-hGH (SEQ ID NO: 13) all deliver similar hGH levels, while Chx¹⁹⁷-SAAG-N3-hGH (SEQ ID NO: 12) delivered a significantly higher level to the serum (FIG. 23 and FIG. 24). Constructs having a cholix carrier truncated at position 197 appeared to improve delivery to the serum relative to a cholix truncated at position 266 (SEQ ID NO: 34). Similarly, Chx¹⁹⁷-hGH (SEQ ID NO: 7), Chx¹⁹⁷-G-hGH (SEQ ID NO: 8), Chx¹⁹⁷-GG-hGH (SEQ ID NO: 9), and Chx¹⁹⁷-GGG-hGH (SEQ ID NO: 10) all delivered higher hGH levels than hGH alone, and Chx¹⁹⁷-GG-hGH (SEQ ID NO: 9) delivered the highest level of hGH to the serum among the four delivery constructs (FIG. 25 and FIG. 26).

### In Vivo Transport Via Serum Analysis Following Sprague Dawley Rat Intrajejunal Administration

Dual pre-cannulated jugular and jejunum female Sprague Dawley rats (250 g and ˜12 weeks of age) were acquired from Charles River Labs (Hollister, CA). Animals were single housed, had access to food and water ad libitum, and were maintained on a 12-hour light/dark cycle with room temperature at 22±2° C. and approximately 50% humidity. On the day of the experiment, 5 hours prior to the procedure, animals were moved to new cages with fresh bedding and no access to food but ad libitum water. The jejunum catheter was flushed with vehicle to ensure the complete dose administration of four different constructs (Chx¹⁹⁷-hGH (SEQ ID NO: 7), Chx¹⁹⁷-SAAG-hGH (SEQ ID NO: 11), Chx¹⁹⁷(ETKTR)-hGH (SEQ ID NO: 13), and Chx¹⁹⁷. SAAG-N3-hGH (SEQ ID NO: 12)) in a final volume of 100 μL. Blood serum samples were collected through the jugular cannulation at the indicated time-points and allowed to clot at RT for <60 min before centrifugation. Patency of the jugular cannulation was checked before dosing and locked with heparin/dextrose after the last blood collection. The jejunal catheter placement and viability were assessed after termination of the study by ensuring cannula insertion and patency in a post-mortem necropsy analysis.

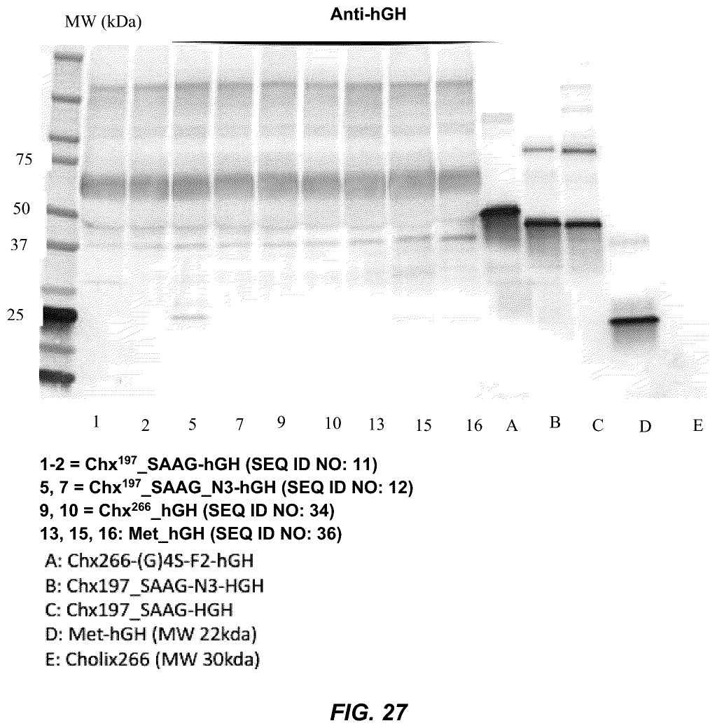

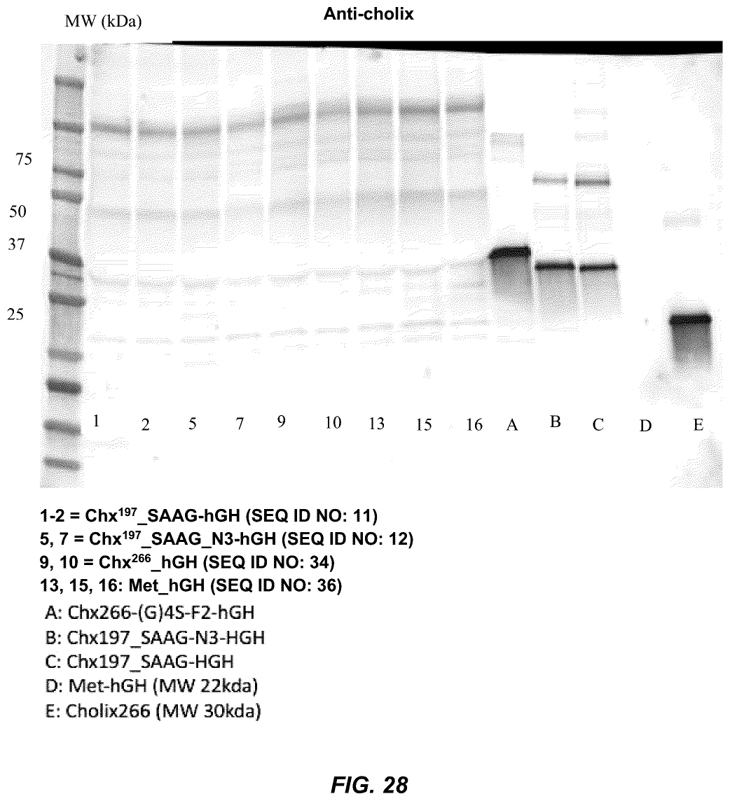

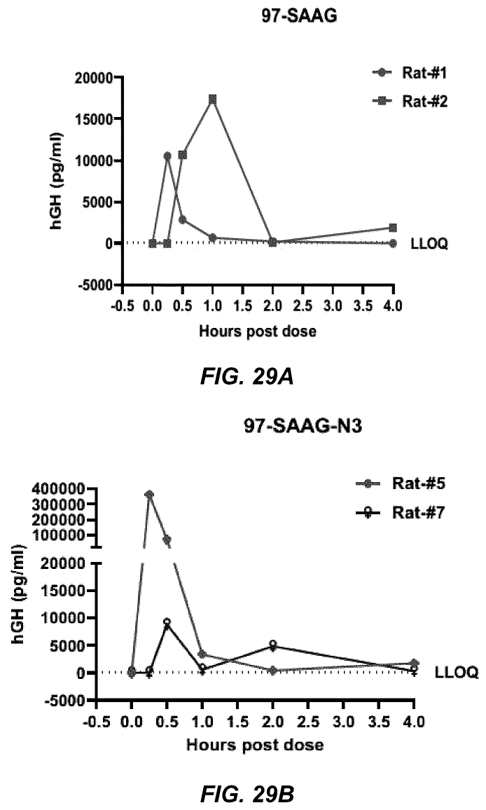

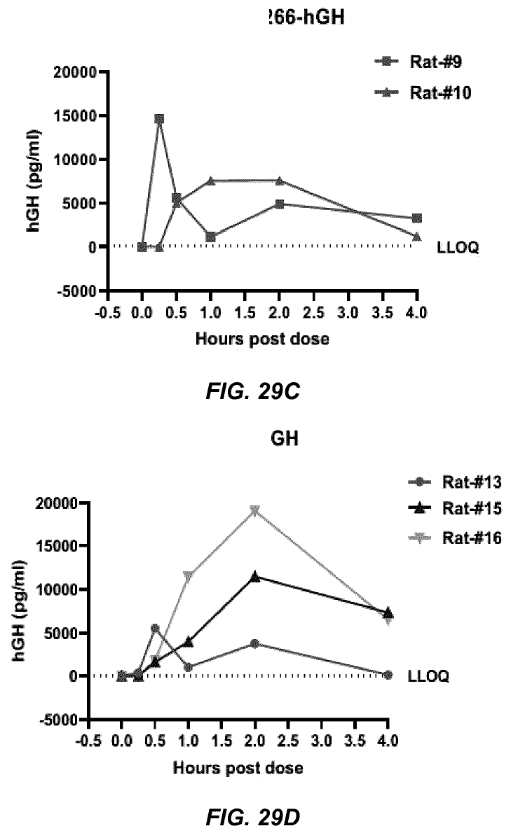

Serum samples from the Sprague Dawley rats involved in this study were obtained at different time points after administration (˜1 mg/kg dose of delivery construct) and analyzed by Western Blot and ELISA for hGH concentration and cholix concentration. The results are shown in FIGS. 27-29D. Specifically, FIG. 27 is a Western blot assessing hGH levels in serum for Chx¹⁹⁷-SAAG-hGH (SEQ ID NO: 11), Chx¹⁹⁷-SAAG-N3-hGH (SEQ ID NO: 12), Chx²⁶⁶-F2-hGH (SEQ ID NO: 34, and hGH (SEQ ID NO: 36), along with additional controls. As shown in FIG. 27, free hGH was observed in rat number 5 (to which was administered Chx¹⁹⁷-SAAG-N3-hGH (SEQ ID NO: 12)). FIG. 28 is a Western blot assessing cholix levels in the serum. No cholix was detected in animal serum by Western blot. FIG. 29A, FIG. 29B, FIG. 29C, and FIG. 29D show the results of ELISA assays of serum in individual animals who were administered Chx¹⁹⁷-SAAG-hGH (SEQ ID NO: 11; FIG. 29A), Chx¹⁹⁷-SAAG-N3-hGH (SEQ ID NO: 12; FIG. 29B), Chx²⁶⁶-F2-hGH (SEQ ID NO: 34; FIG. 29C), and hGH (SEQ ID NO: 36; FIG. 29D). The increase in hGH shown in FIG. 29D lags temporally behind the increases shown in other plots, which is inconsistent with an active transport mechanism. Further, the peak levels of hGH measured in rat number 5 (which received Chx¹⁹⁷-SAAG-N3-hGH (SEQ ID NO: 12)) were approximately 20× higher than the serum levels of hGH measured in rat number sixteen which received hGH construct (SEQ ID NO: 36) without any carrier.

The antibodies used were primary anti-hGH AB (R&D system, Cat. #: AF1067, goat polyclonal); secondary donkey anti-goat IgG, Jackson Immuno., Cat. #: 705-055-147; primary anti-cholix 266 (rabbit polyclonal AB); second: antirabbit AB, Jackson Immuno., cat #111-055-003.

### Example 6—Limited Cleavage in Certain Delivery Constructs

Human 3D in vitro small intestinal tissues (SMI-100) were purchased from MatTek Corporation (Ashland, MA, USA). Following 24 hours incubation at 37° C. and 5% CO₂, transepithelial electric resistance (TEER) was measured for monolayer integrity. Inserts with a TEER>400 Ω·cm2 were used. Dextran (70 kD) was also used as a marker for negative transport and insert integrity. The chambers were washed once with transport buffer (PBS). 100 μl of PBS containing 400 nM of hGH (SEQ ID NO: 35) or Chx²⁶⁶-F2-hGH (SEQ ID NO: 34) was added in apical side and 500 μl of PBS was added to basolateral chambers. The proteins in the basal solution after 2 hours were analyzed by Western blotting for their transport extent and cleavage patterns.

Specifically, samples were separated by gel electrophoresis in a 4-12% NuPAGE gel (BioRad, cat. #5678095). The separated proteins were transferred to PVDF membrane (BioRad, cat. #1704157). Bands positive for hGH (SEQ ID NO: 35) and Chx²⁶⁶-F2-hGH (SEQ ID NO: 34) were detected by goat anti-hGH polyclonal antibody (1:1000, R&D AF1067), followed by AP-conjugated secondary rabbit anti-goat antibody (1:10000, Abcam ab6742). Proteins bands were visualized using AP Western blotting substrate (Promega, Cat. #S3841).

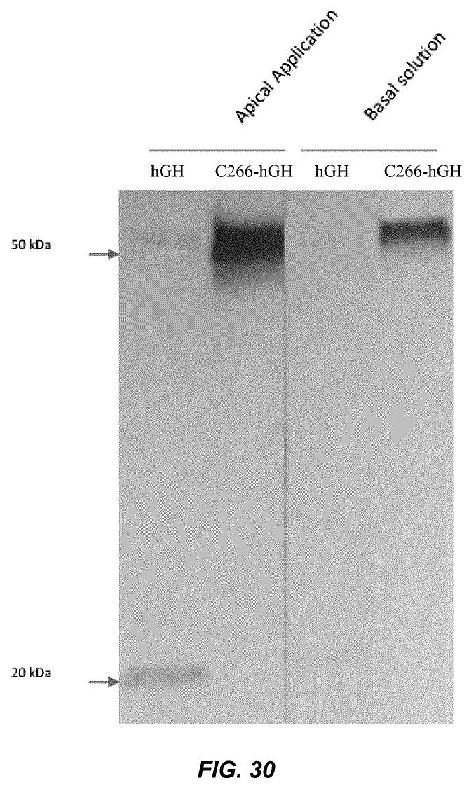

As shown in FIG. 30, low levels of hGH applied to the apical surface of SMI-100 monolayers demonstrate low levels of non-specific leakage to the basal compartment over a 2-hour period. Over same time period, cholix genetically coupled to hGH through a potential furin-cleavable sequence efficiently moves from the apical to basal compartment but fails to be cleaved by furin to release hGH from the cholix²⁶⁶ domain.

### Example 7—the Insertion of Amino Acid Tryptophan (W) in Chx¹⁹⁷-SAAG-N3-hGH Abolished the hGH Release after Transcytosis Across SMI-100 Cells

Human 3D in vitro small intestinal tissues (SMI-100) were purchased from MatTek Corporation (Ashland, MA, USA). Following 24 hours incubation at 37° C. and 5% CO₂, transepithelial electric resistance (TEER) was measured for monolayer integrity. Inserts with a TEER>400 Ω·cm2 were used. Dextran (70 kD) was also used as a marker for negative transport and insert integrity. The chambers were washed once with transport buffer (PBS). 100 μl of PBS containing 400 nM of Chx²⁶⁶-F2-hGH (SEQ ID NO: 34), Chx¹⁹⁷-SAAG-hGH (SEQ ID NO:12) or Chx¹⁹⁷-WSAAG-N3-hGH (SEQ ID NO: 65) was added in apical side and 500 μl of PBS was added to basolateral chambers. The proteins in the basal solution after 60 minutes were analyzed by Western blotting for their cleavage patterns.

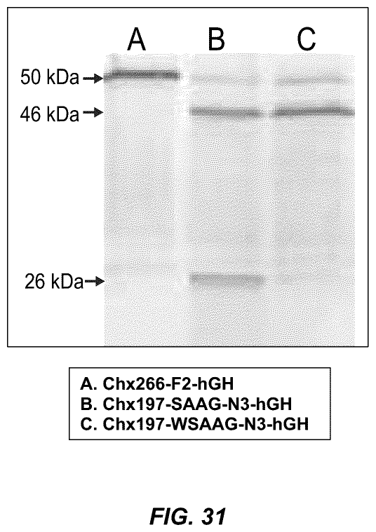

Low levels of hGH in the basal solutions suggested that the cholix delivery construct has a low cleavage efficiency to release hGH. As shown FIG. 31, Chx¹⁹⁷-SAAG-hGH (SEQ ID NO: 12) had a much higher cleavage efficiency than Chx²⁶⁶-F2-hGH (SEQ ID NO: 34) as more hGH was released into the basal solution. The insertion of amino acid tryptophan (W) in Chx¹⁹⁷-WSAAG-N3-hGH (SEQ ID NO: 65) abolished the hGH release.

### Example 8—Furin Cleavage Preferences

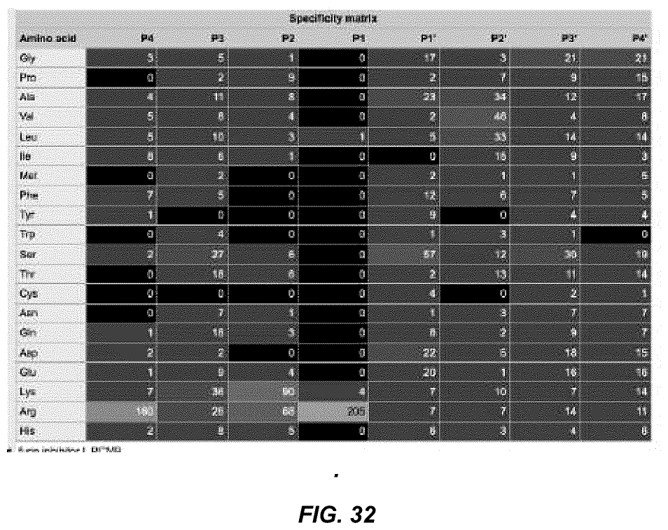

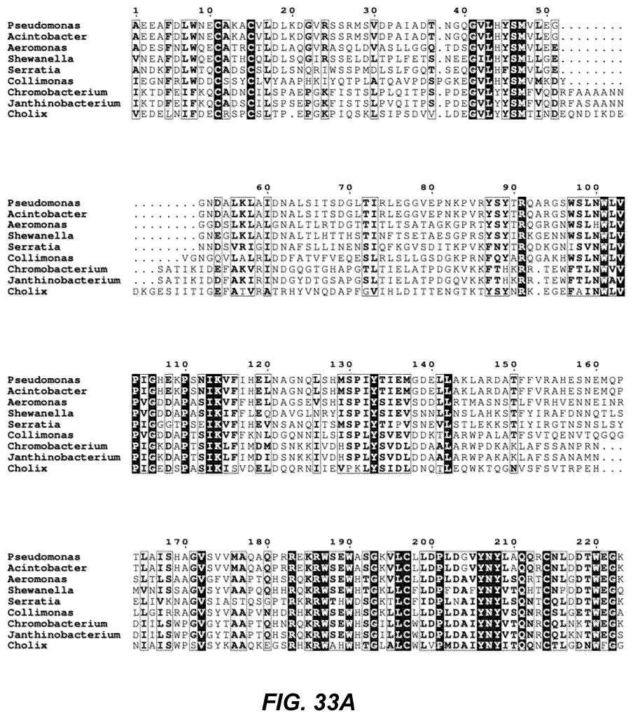

A MEROPS specificity matrix was prepared (FIG. 32) that shows amino acids positioned N-terminal of (P) and C-terminal of (P′) the scissile bond of furin cleavage sites of 205 proteins that are known to be cleaved by furin. Using this sequence analysis, sequences with an enhanced probability for furin cleavage were obtained. Rawlings et al., Nucleic Acids Research 46, D624-D632; ebi.ac.uk/merops/cgi-bin/pepsum?id=S08.071.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-58. (canceled)

59. A furin-cleavable delivery construct for delivery of a heterologous cargo across a polarized epithelial cell via transcytosis, the delivery construct comprising:
a transcytosing element derived from a domain I of a mono-ADP-ribosyl transferase (mART);
a heterologous cargo coupled to the transcytosing element; and
a furin-cleavable cleavage site, wherein the furin-cleavable cleavage site has a scissile bond that is positioned between the transcytosing element and the heterologous cargo such that cleavage of the scissile bond by a furin protease releases the heterologous cargo from the transcytosing element;
wherein the scissile bond of the furin-cleavable cleavage site is positioned such that, upon cleavage of the scissile bond by the furin protease, a released N-terminal fragment comprising the transcytosing element lacks a C-terminal portion of a domain I, lacks a domain II, and lacks a domain III of a mART.

60. The furin-cleavable delivery construct of claim 59, wherein the furin-cleavable cleavage site comprises a contiguous sequence of P4-P3-P2-P1-P1′-P2′-P3′-P4′, wherein P1 is Arg, wherein P2, P3, P4, P1′, P2′, P3′, and P4′ are each independently selected from any naturally occurring amino acid, and wherein the scissile bond of the cleavage site is positioned between P1-P1′.

61. The furin-cleavable delivery construct of claim 60, wherein P2 is Arg or Lys and P4 is Arg.

62. The furin-cleavable delivery construct of claim 60, wherein P3 is selected from the group consisting of histidine, glutamine, lysine, and glutamic acid.

63. The furin-cleavable delivery construct of claim 60, wherein P2′ is selected from the group consisting of Val, Ala, Leu, Ile, Ser, Thr, and Lys.

64. The furin-cleavable delivery construct of claim 60, wherein P3′ is selected from the group consisting of Gly, Ala, Leu, Ser, Thr, Asp, Glu, and Arg.

65. The furin-cleavable delivery construct of claim 60, wherein P4′ is selected from the group consisting of Gly, Pro, Ala, Leu, Ser, Thr, Asp, Glu, Lys, and Arg.

66. The furin-cleavable delivery construct of claim 60, wherein the furin-cleavable cleavage site further comprises a contiguous sequence of P14-P13-P12-P11-P10-P9-P8-P7-P6-P5 positioned immediately N-terminal of P4, wherein P14 is Ser or Gly and wherein P9 is Gln or Pro.

67. The furin-cleavable delivery construct of claim 66, wherein the contiguous sequence of P14-P13-P12-P11-P10-P9-P8-P7-P6-P5 comprises SYKAAQKEGS.

68. The furin-cleavable delivery construct of claim 67, wherein the contiguous sequence of P14-P13-P12-P11-P10-P9-P8-P7-P6-P5 comprises SVVMAQAQPR.

69. The furin-cleavable delivery construct of claim 59, wherein the scissile bond is positioned between amino acid residues 186 and 260 of SEQ ID NO: 43 when the transcytosing element and the furin-cleavable cleavage site are optimally aligned with SEQ ID NO: 43.

70. The furin-cleavable delivery construct of claim 59, wherein the scissile bond is positioned between amino acid residues 186 and 248 of SEQ ID NO: 44 when the transcytosing element and the furin-cleavable cleavage site are optimally aligned with SEQ ID NO: 44.

71. The furin-cleavable delivery construct of claim 59, further comprising a linker positioned between the furin cleavable cleavage site and the heterologous cargo.

72. The furin cleavable-delivery construct of claim 71, wherein the linker is coupled to and positioned N-terminal of the cargo upon cleavage of the scissile bond by the furin protease.

73. The furin-cleavable delivery construct of claim 59, wherein the transcytosing element has at least 80%, at least 85%, at least 90%, at least 95%, at least 98%, at least 99%, or 100% identity to a transcytosing element from a domain I of a Vibrio cholerae, Pseudomonas aeruginosa, Aeromonas, Chromobacterium, Collimonas, Shewanella, Janthinobacterium, Serratia, or Acinetobacter mART.

74. The furin-cleavable delivery construct of claim 59, wherein the delivery construct comprises:
a carrier consisting of SEQ ID NO: 20, SEQ ID NO: 60, SEQ ID NO: 61, or SEQ ID NO: 52, and optionally an N-terminal methionine; and
a linker between the transcytosing element and the heterologous cargo, wherein the linker comprises the amino acid sequence set forth in any one of SEQ ID NOS: 26-32.

75. A method of delivering a cargo across an epithelial membrane, the method comprising:
delivering a furin-cleavable delivery construct to an apical side of an epithelial membrane such that the cargo (1) transcytoses across an epithelial cell of the epithelial membrane and (2) is cleaved from the transcytosing element by a furin protease to deliver the cargo to a basolateral side of the epithelial membrane, wherein the furin-cleavable delivery construct comprises a transcytosing element derived from a domain I of a mono-ADP-ribosyl transferase (mART);
a heterologous cargo coupled to the transcytosing element; and a furin-cleavable cleavage site, wherein the furin-cleavable cleavage site has a scissile bond that is positioned between the transcytosing element and the heterologous cargo such that cleavage of the scissile bond by a furin protease releases the heterologous cargo from the transcytosing element, and wherein the scissile bond of the furin-cleavable cleavage site is positioned such that, upon cleavage of the scissile bond by the furin protease, a released N-terminal fragment comprising the transcytosing element lacks a C-terminal portion of a domain I, lacks a domain II, and lacks a domain III of a mART.

76. A nucleic acid comprising (a) a polynucleotide sequence encoding a furin-cleavable delivery construct for delivery of a heterologous cargo across a polarized epithelial cell via transcytosis, the delivery construct comprising:
a transcytosing element derived from a domain I of a mono-ADP-ribosyl transferase (mART);
a heterologous cargo coupled to the transcytosing element; and
a furin-cleavable cleavage site, wherein the furin-cleavable cleavage site has a scissile bond that is positioned between the transcytosing element and the heterologous cargo such that cleavage of the scissile bond by a furin protease releases the heterologous cargo from the transcytosing element;
wherein the scissile bond of the furin-cleavable cleavage site is positioned such that, upon cleavage of the scissile bond by the furin protease, a released N-terminal fragment comprising the transcytosing element lacks a C-terminal portion of a domain I, lacks a domain II, and lacks a domain III of a mART; or
(b) a reverse complement of said polynucleotide sequence of (a).

77. An expression vector comprising said polynucleotide sequence of claim 76 and a recombinant regulatory sequence operably linked to said polynucleotide sequence.

78. A host cell comprising said expression vector of claim 77.


SEQUENCE LISTING (92 sequences)

SEQ ID NO: 1 - protein, 634 aa, synthetic construct
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAERDARGVMLRVYIPRASLERFYRTNTPLENAEEHITQVIGHSLPLRNEAFTGPESAGGEDETVIGWDMAIHAVAIPSTIPGNAYEELAIDEEAVAKEQSISTKPPYKERKDELK

SEQ ID NO: 2 - protein, 634 aa, synthetic construct
LEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAERDARGVMLRVYIPRASLERFYRTNTPLENAEEHITQVIGHSLPLRNEAFTGPESAGGEDETVIGWDMAIHAVAIPSTIPGNAYEELAIDEEAVAKEQSISTKPPYKERKDELK

SEQ ID NO: 3 - protein, 634 aa, unidentified
VEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAERDARGVMLRVYIPRASLERFYRTNTPLENAEEHITQVIGHSLPLRNEAFTGPESAGGEDETVIGWDMAIHAVAIPSTIPGNAYEELAIDEEAVAKEQSISTKPPYKERKDELK

SEQ ID NO: 4 - protein, 267 aa, synthetic construct
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKG

SEQ ID NO: 5 - protein, 266 aa, synthetic construct
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKG

SEQ ID NO: 6 - protein, 266 aa, synthetic construct
LEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKG

SEQ ID NO: 7 - protein, 395 aa, synthetic construct
MHHHHHHVEDELNIFDECRSPCSLTPEPGKPIQSKLSIPSDVVLDEGVLYYSMTINDEQNDIKDEDKGESIITIGEFATVRATRHYVQQDAPFGVIHLDITTEQGTKTYSYNRKEGEFAINWLVPIGEDSPASIKISVDELDQQRNIIEVPKLYSIDLDQQTLEQWKTQGQVSFSVTRPEHNIAISWPSVSYKAAQKEGSRHKRFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 8 - protein, 396 aa, synthetic construct
MHHHHHHVEDELNIFDECRSPCSLTPEPGKPIQSKLSIPSDVVLDEGVLYYSMTINDEQNDIKDEDKGESIITIGEFATVRATRHYVQQDAPFGVIHLDITTEQGTKTYSYNRKEGEFAINWLVPIGEDSPASIKISVDELDQQRNIIEVPKLYSIDLDQQTLEQWKTQGQVSFSVTRPEHNIAISWPSVSYKAAQKEGSRHKRGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 9 - protein, 397 aa, synthetic construct
MHHHHHHVEDELNIFDECRSPCSLTPEPGKPIQSKLSIPSDVVLDEGVLYYSMTINDEQNDIKDEDKGESIITIGEFATVRATRHYVQQDAPFGVIHLDITTEQGTKTYSYNRKEGEFAINWLVPIGEDSPASIKISVDELDQQRNIIEVPKLYSIDLDQQTLEQWKTQGQVSFSVTRPEHNIAISWPSVSYKAAQKEGSRHKRGGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 10 - protein, 398 aa, synthetic construct
MHHHHHHVEDELNIFDECRSPCSLTPEPGKPIQSKLSIPSDVVLDEGVLYYSMTINDEQNDIKDEDKGESIITIGEFATVRATRHYVQQDAPFGVIHLDITTEQGTKTYSYNRKEGEFAINWLVPIGEDSPASIKISVDELDQQRNIIEVPKLYSIDLDQQTLEQWKTQGQVSFSVTRPEHNIAISWPSVSYKAAQKEGSRHKRGGGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 11 - protein, 399 aa, synthetic construct
MHHHHHHVEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRSAAGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 12 - protein, 411 aa, synthetic construct
MHHHHHHVEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRSAAGGGGSGGGSGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 13 - protein, 408 aa, synthetic construct
MHHHHHHVEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSERTKRGGGSGGGSGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 14 - protein, 388 aa, synthetic construct
VEDELNIFDECRSPCSLTPEPGKPIQSKLSIPSDVVLDEGVLYYSMTINDEQNDIKDEDKGESIITIGEFATVRATRHYVQQDAPFGVIHLDITTEQGTKTYSYNRKEGEFAINWLVPIGEDSPASIKISVDELDQQRNIIEVPKLYSIDLDQQTLEQWKTQGQVSFSVTRPEHNIAISWPSVSYKAAQKEGSRHKRFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 15 - protein, 389 aa, synthetic construct
VEDELNIFDECRSPCSLTPEPGKPIQSKLSIPSDVVLDEGVLYYSMTINDEQNDIKDEDKGESIITIGEFATVRATRHYVQQDAPFGVIHLDITTEQGTKTYSYNRKEGEFAINWLVPIGEDSPASIKISVDELDQQRNIIEVPKLYSIDLDQQTLEQWKTQGQVSFSVTRPEHNIAISWPSVSYKAAQKEGSRHKRGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 16 - protein, 390 aa, synthetic construct
VEDELNIFDECRSPCSLTPEPGKPIQSKLSIPSDVVLDEGVLYYSMTINDEQNDIKDEDKGESIITIGEFATVRATRHYVQQDAPFGVIHLDITTEQGTKTYSYNRKEGEFAINWLVPIGEDSPASIKISVDELDQQRNIIEVPKLYSIDLDQQTLEQWKTQGQVSFSVTRPEHNIAISWPSVSYKAAQKEGSRHKRGGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 17 - protein, 391 aa, synthetic construct
VEDELNIFDECRSPCSLTPEPGKPIQSKLSIPSDVVLDEGVLYYSMTINDEQNDIKDEDKGESIITIGEFATVRATRHYVQQDAPFGVIHLDITTEQGTKTYSYNRKEGEFAINWLVPIGEDSPASIKISVDELDQQRNIIEVPKLYSIDLDQQTLEQWKTQGQVSFSVTRPEHNIAISWPSVSYKAAQKEGSRHKRGGGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 18 - protein, 392 aa, synthetic construct
VEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRSAAGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 19 - protein, 404 aa, synthetic construct
VEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRSAAGGGGSGGGSGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 20 - protein, 197 aa, synthetic construct
VEDELNIFDECRSPCSLTPEPGKPIQSKLSIPSDVVLDEGVLYYSMTINDEQNDIKDEDKGESIITIGEFATVRATRHYVQQDAPFGVIHLDITTEQGTKTYSYNRKEGEFAINWLVPIGEDSPASIKISVDELDQQRNIIEVPKLYSIDLDQQTLEQWKTQGQVSFSVTRPEHNIAISWPSVSYKAAQKEGSRHKR

SEQ ID NO: 21 - protein, 198 aa, synthetic construct
VEDELNIFDECRSPCSLTPEPGKPIQSKLSIPSDVVLDEGVLYYSMTINDEQNDIKDEDKGESIITIGEFATVRATRHYVQQDAPFGVIHLDITTEQGTKTYSYNRKEGEFAINWLVPIGEDSPASIKISVDELDQQRNIIEVPKLYSIDLDQQTLEQWKTQGQVSFSVTRPEHNIAISWPSVSYKAAQKEGSRHKRG

SEQ ID NO: 22 - protein, 199 aa, synthetic construct
VEDELNIFDECRSPCSLTPEPGKPIQSKLSIPSDVVLDEGVLYYSMTINDEQNDIKDEDKGESIITIGEFATVRATRHYVQQDAPFGVIHLDITTEQGTKTYSYNRKEGEFAINWLVPIGEDSPASIKISVDELDQQRNIIEVPKLYSIDLDQQTLEQWKTQGQVSFSVTRPEHNIAISWPSVSYKAAQKEGSRHKRGG

SEQ ID NO: 23 - protein, 200 aa, synthetic construct
VEDELNIFDECRSPCSLTPEPGKPIQSKLSIPSDVVLDEGVLYYSMTINDEQNDIKDEDKGESIITIGEFATVRATRHYVQQDAPFGVIHLDITTEQGTKTYSYNRKEGEFAINWLVPIGEDSPASIKISVDELDQQRNIIEVPKLYSIDLDQQTLEQWKTQGQVSFSVTRPEHNIAISWPSVSYKAAQKEGSRHKRGGG

SEQ ID NO: 24 - protein, 201 aa, synthetic construct
VEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRSAAG

SEQ ID NO: 25 - protein, 213 aa, synthetic construct
VEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRSAAGGGGSGGGSGGGS

SEQ ID NO: 26-28 - skipped (3 entries)

SEQ ID NO: 29 - protein, 4 aa, synthetic construct
SAAG

SEQ ID NO: 30 - protein, 16 aa, synthetic construct
SAAGGGGSGGGSGGGS

SEQ ID NO: 31 - protein, 12 aa, synthetic construct
GGGSGGGSGGGS

SEQ ID NO: 32 - protein, 15 aa, synthetic construct
GGGGSGGGGSGGGGS

SEQ ID NO: 33 - protein, 16 aa, synthetic construct
GGGGGSNLQGGLRQPR

SEQ ID NO: 34 - protein, 473 aa, synthetic construct
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGGGGGSNLQGGLRQPRFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 35 - protein, 191 aa, synthetic construct [sequence omitted]

SEQ ID NO: 36 - protein, 192 aa, synthetic construct
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 37 - protein, 146 aa, synthetic construct
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 38 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 39 - protein, 4 aa, synthetic construct
VEDE

SEQ ID NO: 40 - protein, 4 aa, synthetic construct
VEEA

SEQ ID NO: 41 - protein, 4 aa, synthetic construct
LEEA

SEQ ID NO: 42 - protein, 275 aa, synthetic construct
  VARIANT 1: note = V or L
  VARIANT 3: note = E or D
  VARIANT 4: note = A or E
  VARIANT 6: note = N or K
  VARIANT 16: note = S or L
  VARIANT 21: note = P or L
  VARIANT 24: note = P or Q
  VARIANT 30: note = S or F
  VARIANT 33: note = S or G
  VARIANT 56: note = K or M
  VARIANT 59: note = D or G
  VARIANT 67: note = I or F
  VARIANT 73: note = V or I
  VARIANT 81: note = N or S
  VARIANT 90: note = H or N
  VARIANT 101: note = T or M
  VARIANT 104: note = Y or F
  VARIANT 108: note = E or D
  VARIANT 109: note = G or S
  VARIANT 112: note = A or T
  VARIANT 114: note = N or H
  VARIANT 118: note = P or I
  VARIANT 119: note = I or P
  VARIANT 131: note = V or I
  VARIANT 134: note = L or I
  VARIANT 137: note = Q or K
  VARIANT 160: note = K or E
  VARIANT 161: note = T or N
  VARIANT 166: note = S or F
  VARIANT 168: note = S or A
  VARIANT 174: note = H or Q
  VARIANT 175..185: note = This region may encompass the residues N, S, SIAKQS or SIAKQSIAKQS
  VARIANT 196: note = K or N
  VARIANT 199: note = Q, E, or H
  VARIANT 201: note = E, N, or D
  VARIANT 203: note = S or A
  VARIANT 210: note = H or N
  VARIANT 212: note = H, L, F, or R
  VARIANT 214: note = G or T
  VARIANT 215: note = L or S
  VARIANT 216: note = A or P
  VARIANT 217: note = L, E, or K
  VARIANT 218: note = C or V
  VARIANT 219: note = W, V, or T
  VARIANT 221: note = V or absent
  VARIANT 222: note = P or absent
  VARIANT 223: note = M, I, L, or absent
  VARIANT 224: note = D or absent
  VARIANT 225: note = A or absent
  VARIANT 226: note = I or absent
  VARIANT 227: note = Y or C
  VARIANT 228: note = N or F
  VARIANT 229: note = Y or F
  VARIANT 230: note = I or E
  VARIANT 231: note = T or D
  VARIANT 232: note = Q or P
  VARIANT 233: note = Q, E, or A
  VARIANT 234: note = N, L, or Q
  VARIANT 237: note = L or Y
  VARIANT 239: note = D or E
  VARIANT 240: note = N or D
  VARIANT 242: note = F, H, or Y
  VARIANT 245: note = S or A
  VARIANT 247: note = E or K
  VARIANT 252: note = T or I
  VARIANT 254: note = K, E, or G
  VARIANT 255: note = V or A
  VARIANT 257: note = T or M
  VARIANT 262: note = I or M
  VARIANT 266: note = P, T, or A
  VARIANT 275: note = K, Q, or N
XEXXLXIFDECRSPCXLTPEXGKXIQSKLXIPXDVVLDEGVLYYSMTINDEQNDIXDEXKGESIITXGEFATXRATRHYVXQDAPFGVIXLDITTENGTKXYSXNRKXXEFXIXWLVXXGEDSPASIKISXDEXDQXRNIIEVPKLYSIDLDNQTLEQWXXQGNVXFXVTRPEXXXXXXXXXXXXIAISWPSVSYXAAXKXGXRHKRWAXWXTXXXXXXLXXXXXXXXXXXXXXCTXGXXWXGGXYXTVAGXPXXIXVKQGXEQKXVEQRIHFSX

SEQ ID NO: 43 - protein, 265 aa, Vibrio cholerae
VEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSK

SEQ ID NO: 44 - protein, 252 aa, Pseudomonas aeruginosa
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPE

SEQ ID NO: 45 - protein, 252 aa, Aeromonas hydrophila
ADESFNLWQECATRCTLDLAQGVRASQLDVASLLGGQTDSGVLHYSMVLEEGGDSLKLALGNALTLRTDGTTITLTSATAGKGPRTYSYTRQGRGNWSLHWLVPVGDDAPASIKVFFHELDAGSEVSHISPIYSIEVSDDLLRTMASNSTLFVRHVENNEINRSLTLSAAGVGFVAAPTQHSRQKRWSEWHTGKVLCLLDPLDAVYNYLSQRTCNLGDTWEGKVYRVLAGAPASHDTHIVPTAISHRLHFAK

SEQ ID NO: 46 - protein, 262 aa, Chromobacterium haemolyticum
IKTDFEIFKQCADNCILSPAEPGKFISTSLPLQITPSPDEGVLYYSMFVQDRFAAAANNSATIKIDEFAKVRINDGQGTGHAPGTLTIELATPDGKVKKFTHKRRTEWFTLNWVVPIGKDAPTSIKLFIMDMDSNKKIVDHSPLYSVDLDDAALARWPDKAKLAFSSANPRNDIILSWPGVGYTAAPTQHNRQKRWSEWHSGILLCWLDPLDAIYNYVTQNRCQLNKTWEGKLYQVVAGKPQINEFKPLAKAPIQHRVHFSK

SEQ ID NO: 47 - protein, 257 aa, Collimonas fungivorans
IEGNFRLWDDCSSYCLVYAAPHKIYQTPLATQAVPDSPGKEGVLHYSMVMKDYVGNGQVLALRLDDFATVFVEQESLRLSLLGSDGKPRNFQYARQGAKHWSLNWLVPVGDDAPTSIKVFFKNLDGQNNILSISPLYSVEVDDKTLARWPALATFSVTQENVTQGQGLLGIRRAGVSYVAAPVNHDRHKRWSEWHSGKLLCLLDPLDAIYNYVSQNRCSLGETWEGAIYQTLAGRPVDKYAPPASKPVISQRIHFAK

SEQ ID NO: 48 - protein, 255 aa, Shewanella sp.
VNEAFDLWQECATHCQLDLSQGIRSSELDLTPLFETSNEEGILHYSMLLGEGNEGLKLAIDNALTLHTTHSTINFTSETAESGPRSYSYIRKGENNWSLNWLVPVGDDAPASIKIFFLEQDAVGLNRYISPIYSIEVSNNLLNSLAHKSTFYIRAFDNNQTLSMVNISSAGVSYVAAPQQHHRQKRWSEWHTGKLLCFLDPFDAFYNYVTQHTCNPDDTWEGQIYRVLAGNPATLDTTAPSTTPAVISHRIHFDR

SEQ ID NO: 49 - protein, 262 aa, Janthinobacterium lividum
IKTDFEIFKQCADSCILSPSEPGKFISTSLPVQITPSPDEGVLYYSMFVQDRFASAANNSATIKIDDFAKIRINDGYDTGSAPGSLTIELATPDGQVKKFTHKRRTEWFTLNWAVPIGKDAPTSIKLFIMDIDSNKKIVDHSPLYSVDLDDAALARWPAKAKLAFSSANAMNDIILSWPGVGYTAAPTQHSRQKRWSEWHSGILLCWLDPLDAIYNYVTQNRCQLKNTWEGSLYKVVAGNPQINEFKPVAKAPIQHRVHFSK

SEQ ID NO: 50 - protein, 252 aa, Serratia fonticola
ANDKFDLWTQCADSCSLDLSNQRIWSSPMDLSLFGQTSEQGVLHFSMVLGENNDSVRIGIDNAFSLLINENSIQFKGVSDITKPVKFNYTRQDKGNISVNWLVPIGGGTPSEIKVFIHEVNSANQITSMSPIYTIPVSNEVLSTLEKKSTIYIRGTNSNSLSYELIVKNAGVSIASTQSGPTRKRRWTHWDSGKTLCFIDPLNAIYNYLSQNTCQLDDTWEGKVYLTLYGSPATHDIEMPASPISTRTHFSE

SEQ ID NO: 51 - protein, 258 aa, Acinetobacter baumannii
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKDNNNSTPTVISHRLHFPE

SEQ ID NO: 52 - protein, 186 aa, Pseudomonas aeruginosa
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKR

SEQ ID NO: 53 - protein, 186 aa, Aeromonas hydrophila
ADESFNLWQECATRCTLDLAQGVRASQLDVASLLGGQTDSGVLHYSMVLEEGGDSLKLALGNALTLRTDGTTITLTSATAGKGPRTYSYTRQGRGNWSLHWLVPVGDDAPASIKVFFHELDAGSEVSHISPIYSIEVSDDLLRTMASNSTLFVRHVENNEINRSLTLSAAGVGFVAAPTQHSRQKR

SEQ ID NO: 54 - protein, 195 aa, Chromobacterium haemolyticum
IKTDFEIFKQCADNCILSPAEPGKFISTSLPLQITPSPDEGVLYYSMFVQDRFAAAANNSATIKIDEFAKVRINDGQGTGHAPGTLTIELATPDGKVKKFTHKRRTEWFTLNWVVPIGKDAPTSIKLFIMDMDSNKKIVDHSPLYSVDLDDAALARWPDKAKLAFSSANPRNDIILSWPGVGYTAAPTQHNRQKR

SEQ ID NO: 55 - protein, 190 aa, Collimonas fungivorans
IEGNFRLWDDCSSYCLVYAAPHKIYQTPLATQAVPDSPGKEGVLHYSMVMKDYVGNGQVLALRLDDFATVFVEQESLRLSLLGSDGKPRNFQYARQGAKHWSLNWLVPVGDDAPTSIKVFFKNLDGQNNILSISPLYSVEVDDKTLARWPALATFSVTQENVTQGQGLLGIRRAGVSYVAAPVNHDRHKR

SEQ ID NO: 56 - protein, 186 aa, Shewanella sp.
VNEAFDLWQECATHCQLDLSQGIRSSELDLTPLFETSNEEGILHYSMLLGEGNEGLKLAIDNALTLHTTHSTINFTSETAESGPRSYSYIRKGENNWSLNWLVPVGDDAPASIKIFFLEQDAVGLNRYISPIYSIEVSNNLLNSLAHKSTFYIRAFDNNQTLSMVNISSAGVSYVAAPQQHHRQKR

SEQ ID NO: 57 - protein, 195 aa, Janthinobacterium lividum
IKTDFEIFKQCADSCILSPSEPGKFISTSLPVQITPSPDEGVLYYSMFVQDRFASAANNSATIKIDDFAKIRINDGYDTGSAPGSLTIELATPDGQVKKFTHKRRTEWFTLNWAVPIGKDAPTSIKLFIMDIDSNKKIVDHSPLYSVDLDDAALARWPAKAKLAFSSANAMNDIILSWPGVGYTAAPTQHSRQKR

SEQ ID NO: 58 - protein, 186 aa, Serratia fonticola
ANDKFDLWTQCADSCSLDLSNQRIWSSPMDLSLFGQTSEQGVLHFSMVLGENNDSVRIGIDNAFSLLINENSIQFKGVSDITKPVKFNYTRQDKGNISVNWLVPIGGGTPSEIKVFIHEVNSANQITSMSPIYTIPVSNEVLSTLEKKSTIYIRGTNSNSLSYELIVKNAGVSIASTQSGPTRKRR

SEQ ID NO: 59 - protein, 186 aa, Acinetobacter baumannii; identical to SEQ ID NO: 52

SEQ ID NO: 60 - protein, 197 aa, synthetic construct
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKR

SEQ ID NO: 61 - protein, 197 aa, synthetic construct
LEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKR

SEQ ID NO: 62 - protein, 39 aa, synthetic construct
HGEGTFTSDLSKQMEEEAVRLFIEWLKNGGPSSGAPPPS

SEQ ID NO: 63 - protein, 237 aa, synthetic construct
MVEEALNIFDECRSPCSLTPEPGKPIQSKLSIPSDVVLDEGVLYYSMTINDEQNDIKDEDKGESIITIGEFATVRATRHYVQQDAPFGVIHLDITTEQGTKTYSYNRKEGEFAINWLVPIGEDSPASIKISVDELDQQRNIIEVPKLYSIDLDQQTLEQWKTQGQVSFSVTRPEHNIAISWPSVSYKAAQKEGSRHKRHGEGTFTSDLSKQMEEEAVRLFIEWLKNGGPSSGAPPPS

SEQ ID NO: 64 - protein, 232 aa, synthetic construct
MHHHHHHAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRHGEGTFTSDLSKQMEEEAVRLFIEWLKNGGPSSGAPPPS

SEQ ID NO: 65 - protein, 412 aa, synthetic construct
MHHHHHHVEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWSAAGGGGSGGGSGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 66 - protein, 225 aa, synthetic construct
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRHGEGTFTSDLSKQMEEEAVRLFIEWLKNGGPSSGAPPPS

SEQ ID NO: 67 - protein, 405 aa, synthetic construct
VEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWSAAGGGGSGGGSGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 68 - protein, 236 aa, synthetic construct
VEEALNIFDECRSPCSLTPEPGKPIQSKLSIPSDVVLDEGVLYYSMTINDEQNDIKDEDKGESIITIGEFATVRATRHYVQQDAPFGVIHLDITTEQGTKTYSYNRKEGEFAINWLVPIGEDSPASIKISVDELDQQRNIIEVPKLYSIDLDQQTLEQWKTQGQVSFSVTRPEHNIAISWPSVSYKAAQKEGSRHKRHGEGTFTSDLSKQMEEEAVRLFIEWLKNGGPSSGAPPPS

SEQ ID NO: 69 - protein, 10 aa, synthetic construct
SYKAAQKEGS

SEQ ID NO: 70 - protein, 10 aa, synthetic construct
SVVMAQAQPR

SEQ ID NO: 71 - protein, 20 aa, synthetic construct
KEGSRHKRWAHWHTGLALCW

SEQ ID NO: 72 - protein, 20 aa, synthetic construct
KEGSRHKRSAAGFPTIPLSR

SEQ ID NO: 73 - protein, 20 aa, synthetic construct
KEGSRHKRSAAGGGGSGGGS

SEQ ID NO: 74 - protein, 20 aa, synthetic construct
EGSERTKRGGGSGGGSGGGS

SEQ ID NO: 75 - protein, 223 aa, Pseudomonas sp.
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGK

SEQ ID NO: 76 - protein, 223 aa, Acinetobacter sp.; identical to SEQ ID NO: 75

SEQ ID NO: 77 - protein, 223 aa, Aeromonas sp.
ADESFNLWQECATRCTLDLAQGVRASQLDVAPLLGGQTDSGVLHYSMVLEEGGDSLKLALGNALTLRTDGTTITLTSATAGKGPRTYSYTRQGRGNWSLHWLVPVGDDAPASIKVFFHELDAGSEVSHISPIYSIEVSDDLLRTMASNSTLFVRHVENNEINRSLTLSAAGVGFVAAPTQHSRQKRWSEWHTGKVLCLLDPLDAVYNYLSQRTCNLGDTWEGK

SEQ ID NO: 78 - protein, 223 aa, Shewanella sp.
VNEAFDLWQECATHCQLDLSQGIRSSELDLTPLFETSNEEGILHYSMLLGEGNEGLKLAIDNALTLHTTHSTINFTSETAESGPRSYSYIRKGENNWSLNWLVPVGDDAPASIKIFFLEQDAVGLNRYISPIYSIEVSNNLLNSLAHKSTFYIRAFDNNQTLSMVNISSAGVSYVAAPQQHHRQKRWSEWHTGKLLCFLDPFDAFYNYVTQHTCNPDDTWEGQ

SEQ ID NO: 79 - protein, 223 aa, Serratia sp.
ANDKFDLWTQCADSCSLDLSNQRIWSSPMDLSLFGQTSEQGVLHFSMVLGENNDSVRIGIDNAFSLLINENSIQFKGVSDITKPVKFNYTRQDKGNISVNWLVPIGGGTPSEIKVFIHEVNSANQITSMSPIYTIPVSNEVLSTLEKKSTIYIRGTNSNSLSYELIVKNAGVSIASTQSGPTRKRRWTHWDSGKTLCFIDPLNAIYNYLSQNTCQLDDTWEGK

SEQ ID NO: 80 - protein, 227 aa, Collimonas sp.
IEGNFRLWDDCSSYCLVYAAPHKIYQTPLATQAVPDSPGKEGVLHYSMVMKDYVGNGQVLALRLDDFATVFVEQESLRLSLLGSDGKPRNFQYARQGAKHWSLNWLVPVGDDAPTSIKVFFKNLDGQNNILSISPLYSVEVDDKTLARWPALATFSVTQENVTQGQGLLGIRRAGVSYVAAPVNHDRHKRWSEWHSGKLLCLLDPLDAIYNYVSQNRCSLGETWEGA

SEQ ID NO: 81 - protein, 232 aa, Chromobacterium sp.
IKTDFEIFKQCADNCILSPAEPGKFISTSLPLQITPSPDEGVLYYSMFVQDRFAAAANNSATIKIDEFAKVRINDGQGTGHAPGTLTIELATPDGKVKKFTHKRRTEWFTLNWVVPIGKDAPTSIKLFIMDMDSNKKIVDHSPLYSVDLDDAALARWPDKAKLAFSSANPRNDIILSWPGVGYTAAPTQHNRQKRWSEWHSGILLCWLDPLDAIYNYVTQNRCQLNKTWEGK

SEQ ID NO: 82 - protein, 232 aa, Janthinobacterium sp.
IKTDFEIFKQCADSCILSPSEPGKFISTSLPVQITPSPDEGVLYYSMFVQDRFASAANNSATIKIDDFAKIRINDGYDTGSAPGSLTIELATPDGQVKKFTHKRRTEWFTLNWAVPIGKDAPTSIKLFIMDIDSNKKIVDHSPLYSVDLDDAALARWPAKAKLAFSSANAMNDIILSWPGVGYTAAPTQHSRQKRWSEWHSGILLCWLDPLDAIYNYVTQNRCQLKNTWEGS

SEQ ID NO: 83 - protein, 234 aa, unidentified
VEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGG

SEQ ID NO: 84 - protein, 172 aa, Aeromonas sp.
SLTLSAAGVGFVAAPTQHSRQKRWSEWHTGKVLCLLDPLDAVYNYLSQRTCNLGDTWEGKVYRVLAGAPASHDTHIVPTAISHRLHFAKGDGLAALTTHQVCAIPLESLARSRQPRGWEELSQCGYPVHNLVTLYLLTRLPWSQLDTVITQALANTTPEDGSTPRGQLAQAI

SEQ ID NO: 85 - protein, 169 aa, Shewanella sp.
MVNISSAGVSYVAAPQQHHRQKRWSEWHTGKLLCFLDPFDAFYNYVTQHTCNPDDTWEGQIYRVLAGNPATLDTTAPSTTPAVISHRIHFDRGNSLASLTAHQVCGIPLESLARTRHPRGWEELNNCGYPVRNLVSLFILARLSWDRVEQVIHNALTNPTPGNALDDAI

SEQ ID NO: 86 - protein, 166 aa, Pseudomonas sp.
TLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAI

SEQ ID NO: 87 - protein, 172 aa, Acinetobacter sp.
TLAISHAGVSVVMAQAQPRREKRWSEWASGKVLCLLDPLDGVYNYLAQQRCNLDDTWEGKIYRVLAGNPAKHDLDIKDNNNSTPTVISHRLHFPEGGSLAALTAHQACHLPLEAFTRHRQPRGWEQLEQCGYPVQRLVALYLAARLSWNQVDQVIRNALASPGSGGDLGEAI

SEQ ID NO: 88 - protein, 166 aa, Serratia sp.
ELIVKNAGVSIASTQSGPTRKRRWTHWDGGKTLCFIDPLNAIYNYLSQNTCQLDDTWEGKVYLTLYGSPATHDIEMPASPISTRTHFSEGGSLAALTAHRVCAIPFETFTRHRQPRGWEDLEKCGYPVHSLIAFYIAARHSWSQVDQVIQNALDNQESDSDLDRAI

SEQ ID NO: 89 - protein, 174 aa, Collimonas sp.
LLGIRRAGVSYVAAPVNHDRHKRWSEWHSGKLLCLLDPLDAIYNYVSQNRCSLGETWEGAIYQTLAGRPVDKYAPPASKPVISQRIHFAKGNALEALTSHRVCGIPLESLARRRKPRGWEEWSSCGNPAANFVALYIATRLPFDQFRQVIHNLVHGQAVAAPDPVPLDALRTAV

SEQ ID NO: 90 - protein, 175 aa, Chromobacterium sp.
DIILSWPGVGYTAAPTQHNRQKRWSEWHSGILLCWLDPLDAIYNYVTQNRCQLNKTWEGKLYQVVAGKPQINEFKPLAKAPIQHRVHFSKENALGALSAHRVCGIPLESLARSRQPRGWEELSACGYRVESIVGLYIATRLSFDRFRQVVDDLIHSRPVSGAQDPEALEQLGTAV

SEQ ID NO: 91 - protein, 175 aa, Janthinobacterium sp.
DIILSWPGVGYTAAPTQHSRQKRWSEWHSGILLCWLDPLDAIYNYVTQNRCQLKNTWEGSLYKVVAGNPQINEFKPVAKAPIQHRVHFSKENALGALSAHRVCGIPLESLARSRQPRGWEELSACGFRVENIVGLYIATRLSFDRFRQVVEDLIHSRPVSGAQDPVALEQLGSVV

SEQ ID NO: 92 - protein, 172 aa, unidentified
IAISWPSVSYKAAQKEGSRHKRWAHWHTGIALCWLVPMDAIYNYITQQNCTLGDNWFGSSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPECAERLSDLRRIN